(19) United States
(12) Patent Application Publication
(10) Pub. No.: US 2006/0183754 A1
(43) Pub. Date: Aug. 17, 2006

(54) Amide bridged piphenyl or biazaphenyl derivatives

(76) Inventors: Jean Ackermann, Riehen (CH); Johannes Aebi, Basel (CH); Alfred Binggeli, Binningen (CH); Uwe Grether, Efringen-Kirchen (DE); Bernd Kuhn, Liestal (CH); Hans-Peter Maerki, Basel (CH); Markus Meyer, Neuenburg (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/352,099

(22) Filed: Feb. 10, 2006

(30) Foreign Application Priority Data
Feb. 15, 2005 (EP) 05101129.4

(51) Int. Cl. A61K 31/505; A61K 31/44; A61K 31/192; C07D 239/42; C07D 213/55

(52) U.S. Cl. 514/256; 514/357; 514/571; 544/335; 546/335; 562/465

(57) ABSTRACT

This invention is concerned with compounds of the formula 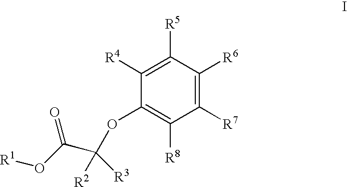
wherein one of R⁵, R⁶ and R⁷ is 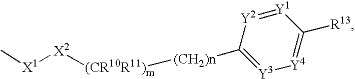
and R¹ to R¹³, X¹, X², m and n are defined in the description, and all pharmaceutically acceptable salts and/or esters thereof. The invention further relates to pharmaceutical compositions containing such compounds, to a process for their preparation and to their use for the treatment and/or prevention of diseases which are modulated by PPARδ and/or PPARα agonists.

### PRIORITY TO RELATED APPLICATIONS

This application claims the benefit of European Application No. 05101129.4, filed Feb. 15, 2005, which is hereby incorporated by reference in its entirety.

### FIELD OF THE INVENTION

The present invention is directed to novel phenyl derivatives, their manufacture, pharmaceutical compositions containing them and their use as medicaments. The active compounds of the present invention are useful as lipid modulators and insulin sensitizers.

In a preferred embodiment, the present invention is directed to compounds of the general formula 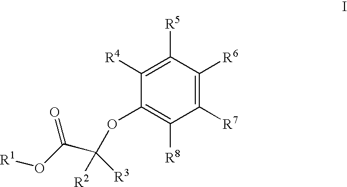
and pharmaceutically acceptable salts and esters thereof.

It has been found that compounds of formula I are PPAR activators.

All documents cited or relied upon herein are expressly incorporated herein by reference.

### BACKGROUND OF THE INVENTION

Peroxisome Proliferator Activated Receptors (PPARs) are members of the nuclear hormone receptor superfamily. The PPARs are ligand-activated transcription factors that regulate gene expression and control multiple metabolic pathways. Three subtypes have been described: PPARα, PPARδ (also known as PPARβ), and PPARγ. PPARδ is ubiquitously expressed. PPARα is predominantly expressed in the liver, kidney and heart. There are at least two major isoforms of PPARγ. PPARγ1 is expressed in most tissues, and the longer isoform, PPARγ2 is almost exclusively expressed in adipose tissue. The PPARs modulate a variety of physiological responses including regulation of glucose- and lipid-homeostasis and metabolism, energy balance, cell differentiation, inflammation and cardiovascular events.

Approximately half of all patients with coronary artery disease have low concentrations of plasma HDL cholesterol. The atheroprotective function of HDL was first highlighted almost 25 years ago and stimulated exploration of the genetic and environmental factors that influence HDL levels. The protective function of HDL comes from its role in a process termed reverse cholesterol transport. HDL mediates the removal of cholesterol from cells in peripheral tissues including those in the atherosclerotic lesions of the arterial wall. HDL then delivers its cholesterol to the liver and sterol-metabolizing organs for conversion to bile and elimination. Data from the Framingham study showed that HDL-C levels are predictive of coronary artery disease risk independently of LDL-C levels. The estimated age-adjusted prevalence among Americans age 20 and older who have HDL-C of less than 35 mg/dl is 16% (males) and 5.7% (females). A substantial increase of HDL-C is currently achieved by treatment with niacin in various formulations. However, the substantial side-effects limit the therapeutic potential of this approach.

As many as 90% of the 14 million diagnosed type 2 diabetic patients in the US are overweight or obese, and a high proportion of type 2 diabetic patients have abnormal concentrations of lipoproteins. The prevalence of total cholesterol >240 mg/dl is 37% in diabetic men and 44% in women. The respective rates for LDL-C>160 mg/dl are 31% and 44%, respectively, and for HDL-C<35 mg/dl 28% and 11%, respectively. Diabetes is a disease in which a patient's ability to control glucose levels in blood is decreased because of partial impairment in response to the action of insulin. Type II diabetes (T2D) is also called non-insulin dependent diabetes mellitus (NIDDM) and afflicts 80-90% of all diabetic patients in developed countries. In T2D, the pancreatic Islets of Langerhans continue to produce insulin. However, the target organs for insulin action, mainly muscle, liver and adipose tissue, exhibit a profound resistance to insulin stimulation. The body continues to compensate by producing unphysiologically high levels of insulin, which ultimately decreases in later stage of disease, due to exhaustion and failure of pancreatic insulin-producing capacity. Thus T2D is a cardiovascular-metabolic syndrome associated with multiple comorbidities including insulin resistance, dyslipidemia, hypertension, endothelial dysfunction and inflammatory atherosclerosis.

First line treatment for dyslipidemia and diabetes generally involves a low-fat and low-glucose diet, exercise and weight loss. However, compliance can be moderate, and as the disease progresses, treatment of the various metabolic deficiencies becomes necessary with e.g. lipid-modulating agents such as statins and fibrates for dyslipidemia and hypoglycemic drugs, e.g. sulfonylureas or metformin for insulin resistance. A promising new class of drugs has recently been introduced that resensitizes patients to their own insulin (insulin sensitizers), thereby restoring blood glucose and triglyceride levels to normal, and in many cases, obviating or reducing the requirement for exogenous insulin. Pioglitazone (Actos™) and rosiglitazone (Avandia™) belong to the thiazolidinedione (TZD) class of PPARγ-agonists and were the first in their class to be approved for NIDDM in several countries. These compounds, however, suffer from side effects, including rare but severe liver toxicity (as seen with troglitazone). They also increase body weight in patients. Therefore, new, more efficacious drugs with greater safety and lower side effects are urgently needed. Recent studies provide evidence that agonism of PPARδ and/or PPARα would result in compounds with enhanced therapeutic potential, i.e. such compounds should improve the lipid profile, with a superior effect on HDL-C raising compared to current treatments and with additional positive effects on normalization of insulin-levels (Oliver et al; Proc Nat Acad Sci USA 2001; 98: 5306-11). Recent observations also suggest that there is a independent PPARα mediated effect on insulin-sensitization in addition to its well known role in reducing triglycerides (Guerre-Millo et al; J Biol Chem 2000; 275: 16638-16642). Thus selective PPARα agonists, selective PPARδ agonists or PPARα/δ co-agonists, optionally with additional moderate PPARγ agonists, may show superior therapeutic efficacy without the side-effects such as the weight gain seen with pure PPARγ agonists.

### SUMMARY OF THE INVENTION

In one embodiment of the present invention, provided is a compound of the formula (I): 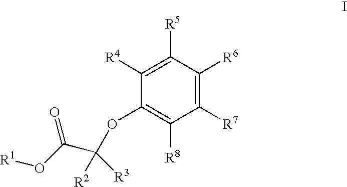
wherein:
R¹ is hydrogen or C₁₋₇-alkyl;
R² and R³ independently from each other are hydrogen or C₁₋₇-alkyl,
R⁴ and R⁸ independently from each other are selected from the group consisting of hydrogen, C₁₋₇-alkyl, C₃₋₇-cycloalkyl, halogen, C₁₋₇-alkoxy-C₁₋₇-alkyl,
C₂₋₇-alkenyl, C₂₋₇-alkinyl, fluoro-C₁₋₇-alkyl, cyano-C₁₋₇-alkyl and cyano;
R⁵, R⁶ and R⁷ independently from each other are selected from the group consisting of hydrogen, C₁₋₇-alkyl, C₃₋₇-cycloalkyl, halogen, C₁₋₇-alkoxy-C₁₋₇-alkyl,
C₂₋₇-alkenyl, C₂₋₇-alkinyl, fluoro-C₁₋₇-alkyl, cyano-C₁₋₇-alkyl and cyano;
and one of R⁵, R⁶ and R⁷ is 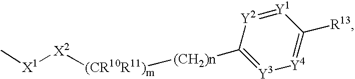
wherein
X¹ is selected from the group consisting of
—(CR¹⁴R¹⁵), —(CR¹⁴R¹⁵)CH₂—, —CH₂(CR¹⁴CR¹⁵)—, —CH₂CH₂CH₂—,
—(CR¹⁴R¹⁵)CH₂CH₂—, —CH₂(CR¹⁴R¹⁵)CH₂—, —CH₂CH₂(CR¹⁴R¹⁵)—,
—CH₂CH₂CH₂CH₂—, —(CR¹⁴R¹⁵)CH₂CH₂CH₂—, —CH₂(CR⁴R¹⁵)CH₂CH₂—,
—CH₂CH₂(CR¹⁴R¹⁵)CH₂—, and —CH₂CH₂CH₂(CR¹⁴R¹⁵)—,
or, in addition,
X¹ is selected from the group consisting of
—OCH₂—, —O(CR¹⁴R¹⁵)—, —OCH₂CH₂—, —O(CR¹⁴H)CH₂—,
—OCH₂(CR¹⁴R¹⁵)—, —OCH₂CH₂CH₂—, —O(CR¹⁴H)CH₂CH₂—,
—OCH₂(CR¹⁴R¹⁵)CH₂—, and —OCH₂CH₂(CR¹⁴R¹⁵)—,
when X² is —CONR⁹—; or
X¹ is selected from the group consisting of
—OCH₂CH₂—, —O(CR¹⁴H)CH₂—, —OCH₂(CR¹⁴R¹⁵)—,
—OCH₂CH₂CH₂—, —O(CR¹⁴H)CH₂CH₂—, —OCH₂(CR¹⁴R¹⁵)CH₂—, and
—OCH₂CH₂(CR¹⁴R¹⁵)—, when X² is —NR⁹CO—,
X² is —NR⁹CO— or —CONR⁹—;
R⁹ is selected from the group consisting of hydrogen, C₁₋₇-alkyl, C₃₋₇-cycloalkyl, fluoro-C₁₋₇-alkyl, hydroxy-C₂₋₇-alkyl, and C₁₋₇-alkoxy-C₂₋₇-alkyl;
Y¹, Y², Y³ and Y⁴ are N or C—R¹², whereas none, one or two of Y¹, Y², Y³ and Y⁴ are N and the other ones are C—R¹²;
R¹⁰ is selected from the group consisting of C₁₋₇-alkyl, C₃₋₇-cycloalkyl, fluoro-C₁₋₇-alkyl, and C₁₋₇-alkoxy-C₁₋₇-alkyl;
R¹¹ is selected from the group consisting of hydrogen, C₁₋₇-alkyl, and
C₁₋₇-alkoxy-C₁₋₇-alkyl;
R¹² independently from each other in each occurrence is selected from the group consisting of hydrogen, C₁₋₇-alkyl, C₃₋₇-cycloalkyl, fluoro-C₁₋₇-alkyl, C₁₋₇-alkoxy-C₁₋₇-alkyl, hydroxy-C₁₋₇-alkyl, C₁₋₇-alkylthio-C₁₋₇-alkyl, carboxy-C₁₋₇-alkoxy-C₁₋₇-alkyl, carboxy, carboxy-C₁₋₇-alkyl, mono- or di-C₁₋₇-alkyl-amino-C₁₋₇-alkyl,
C₁₋₇-alkanoyl-C₁₋₇-alkyl, C₂₋₇-alkenyl, and C₂₋₇-alkinyl;
R¹³ is aryl or heteroaryl;
R¹⁴ is selected from the group consisting of C₁₋₇-alkyl, C₃₋₇-cycloalkyl, fluoro-C₁₋₇-alkyl, and C₁₋₇-alkoxy-C₁₋₇-alkyl;
R¹⁵ is selected from the group consisting of hydrogen, C₁₋₇-alkyl, C₃₋₇-cycloalkyl, fluoro-C₁₋₇-alkyl, and C₁₋₇-alkoxy-C₁₋₇-alkyl;
m is 0 or 1;
n is 0, 1, 2 or 3; and
all pharmaceutically acceptable salts and/or esters thereof.

In another embodiment of the present invention, provided is a process for the manufacture of compounds according to formula (I), comprising the steps of: reacting a compound of formula 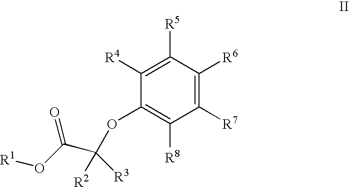
wherein R¹ is C₁₋₇-alkyl, R² to R⁸ are as defined above and one of R⁵, R⁶ or R⁷ is —X¹—COOH,
with a compound of formula 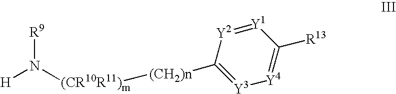
wherein Y¹ to Y⁴, R⁹, R¹⁰, R¹¹, R¹³, m and n are as defined above,
to obtain a compound of formula 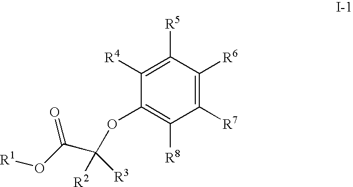
wherein one of R⁵, R⁶ and R⁷ is 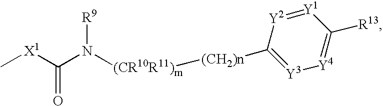
and wherein R¹ is C₁₋₇-alkyl and X¹, Y¹ to Y⁴, R² to R¹³ and m and n are as defined above,
and optionally hydrolyzing the ester group to obtain a compound of formula I-1, wherein R¹ is hydrogen;
or, alternatively,
reacting a compound of formula 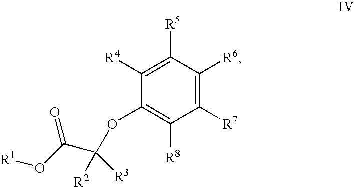
wherein R¹ is C₁₋₇-alkyl, R² to R⁸ are as defined above and one of R⁵, R⁶ or R⁷ is —X¹—NHR⁹, wherein X¹ and R⁹ are as defined above,
with a compound of formula 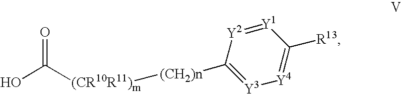
wherein Y¹ to Y⁴, R¹⁰, R¹¹, R¹³, m and n are as defined above,
to obtain a compound of formula 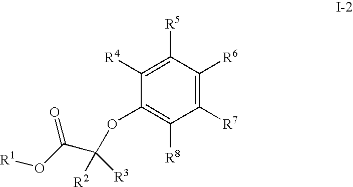
wherein one of R⁵, R⁶ and R⁷ is 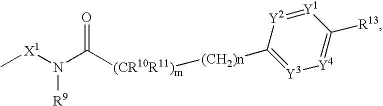
and wherein R¹ is C₁₋₇-alkyl and X¹, Y¹ to Y⁴, R² to R¹³ and m and n are as defined above,
and optionally hydrolyzing the ester group to obtain a compound of formula I-2, wherein R¹ is hydrogen.

In a further embodiment of the present invention, provided is a pharmaceutical composition, comprising a therapeutically effective amount of a compound according to formula (I) and a pharmaceutically acceptable carrier and/or adjuvant.

In a still another embodiment of the present invention, provided is a method for the treatment and/or prevention of diseases which are modulated by PPARδ and/or PPARα agonists, which method comprising the step of administering a therapeutically effective amount of a compound according to formula (I) to a human being or animal in need thereof.

### DETAILED DESCRIPTION

The novel compounds of the present invention exceed the compounds known in the art, inasmuch as they bind to and selectively activate PPARα or coactivate PPARα and PPARδ simultaneously and very efficiently, and with much improved pharmacokinetic properties. Therefore, these compounds combine the anti-dyslipidemic and anti-glycemic effects of PPARα and PPARδ activation, and optionally have an additional moderate effect on PPARγ reinforcing their anti-glycemic potential. Consequently, HDL cholesterol is increased, triglycerides are lowered (=improved lipid profile) and plasma glucose and insulin are reduced (=insulin sensitization). In addition, such compounds may also lower LDL cholesterol, decrease blood pressure and counteract inflammatory atherosclerosis. Furthermore, such compounds may also be useful for treating inflammatory diseases such as rheumatoid arthritis, osteoarthritis, and psoriasis. Since multiple facets of combined dyslipidemia and the T2D disease syndrome are addressed by PPARα or δ-selective agonists and PPARδ and α coagonists, they are expected to have an enhanced therapeutic potential compared to the compounds already known in the art.

The compounds of the present invention further exhibit improved pharmacological properties compared to known compounds.

Unless otherwise indicated the following definitions are set forth to illustrate and define the meaning and scope of the various terms used to describe the invention herein.

The term “alkyl”, alone or in combination with other groups, refers to a branched or straight-chain monovalent saturated aliphatic hydrocarbon radical of one to twenty carbon atoms, preferably one to sixteen carbon atoms, more preferably one to ten carbon atoms.

The term “lower alkyl” or “C₁₋₇-alkyl”, alone or in combination with other groups, refers to a branched or straight-chain monovalent alkyl radical of one to seven carbon atoms, preferably one to four carbon atoms. This term is further exemplified by such radicals as methyl, ethyl, n-propyl, isopropyl, n-butyl, s-butyl, t-butyl and the groups specifically exemplified herein.

The term “lower alkenyl” or “C₂₋₇-alkenyl”, alone or in combination, signifies a straight-chain or branched hydrocarbon residue comprising an olefinic bond and up to 7, preferably up to 6, particularly preferred up to 4 carbon atoms. Examples of alkenyl groups are ethenyl, 1-propenyl, 2-propenyl, isopropenyl, 1-butenyl, 2-butenyl, 3-butenyl and isobutenyl. A preferred example is 2-propenyl.

The term “lower alkinyl” or “C₂₋₇-alkinyl”, alone or in combination, signifies a straight-chain or branched hydrocarbon residue comprising a triple bond and up to 7, preferably up to 6, particularly preferred up to 4 carbon atoms. Examples of alkinyl groups are ethinyl, 1-propinyl, or 2-propinyl.

The term “halogen” refers to fluorine, chlorine, bromine and iodine.

The term “fluoro-lower alkyl” or “fluoro-C₁₋₇-alkyl” refers to lower alkyl groups which are mono- or multiply substituted with fluorine. Examples of fluoro-lower alkyl groups are e.g. —CF₃, —CH₂CF₃, —CH(CF₃)₂ and the groups specifically exemplified herein.

The term “alkoxy” refers to the group R′—O—, wherein R′ is alkyl. The term “lower-alkoxy” or “C₁₋₇-alkoxy” refers to the group R′—O—, wherein R′ is lower-alkyl. Examples of lower-alkoxy groups are e.g. methoxy, ethoxy, propoxy, isopropoxy, butoxy, isobutoxy and hexyloxy. Preferred are the lower-alkoxy groups specifically exemplified herein.

The term “lower fluoroalkoxy” or “fluoro-C₁₋₇-alkoxy” refers to lower alkoxy groups as defined above which are mono- or multiply substituted with fluorine. Examples of lower fluoroalkoxy groups are e.g. —OCF₃, and —OCH₂CF₃.

The term “alkylthio” refers to the group R′—S—, wherein R′ is alkyl. The term “lower-alkylthio” or “C₁₋₇-alkylthio” refers to the group R′—S—, wherein R′ is lower-alkyl. Examples of C₁₋₇-alkylthio groups are e.g. methylthio or ethylthio. Preferred are the lower-alkylthio groups specifically exemplified herein.

The term “mono- or di-C₁₋₇-alkyl-amino” refers to an amino group, which is mono- or disubstituted with C₁₋₇-alkyl. A mono-C₁₋₇-alkyl-amino group includes for example methylamino or ethylamino. The term “di-C₁₋₇-alkyl-amino” includes for example dimethylamino, diethylamino or ethylmethylamino. Preferred are the mono- or di-C₁₋₇-alkylamino groups specifically exemplified herein.

The term “carboxy-lower alkyl” or “carboxy-C₁₋₇-alkyl” refers to lower alkyl groups which are mono- or multiply substituted with a carboxy group (—COOH). Examples of carboxy-lower alkyl groups are e.g. —CH₂—COOH (carboxymethyl), —(CH₂)₂—COOH (carboxyethyl) and the groups specifically exemplified herein.

The term “alkanoyl” refers to the group R′—CO—, wherein R′ is alkyl. The term “lower-alkanoyl” or “C₁₋₇-alkanoyl” refers to the group R′—O—, wherein R′ is lower-alkyl. Examples of lower-alkanoyl groups are e.g. ethanoyl (acetyl) or propionyl. Preferred are the lower-alkanoyl groups specifically exemplified herein.

The term “cycloalkyl” or “C₃₋₇-cycloalkyl” denotes a saturated carbocyclic group containing from 3 to 7 carbon atoms, such as cyclopropyl, cyclobutyl, cyclopentyl, cyclohexyl or cycloheptyl.

The term “aryl” relates to the phenyl or naphthyl group, preferably the phenyl group, which can optionally be mono- or multiply-substituted, particularly mono- or di-substituted by halogen, hydroxy, CN, CF₃, NO₂, NH₂, N(H, lower-alkyl), N(lower-alkyl)₂, carboxy, aminocarbonyl, lower-alkyl, lower fluoro-alkyl, lower-alkoxy, lower fluoro-alkoxy, aryl and/or aryloxy. Preferred substituents are halogen, CF₃, OCF₃, lower-alkyl and/or lower-alkoxy. Preferred are the specifically exemplified aryl groups.

The term “heteroaryl” refers to an aromatic 5- or 6-membered ring which can comprise 1, 2 or 3 atoms selected from nitrogen, oxygen and/or sulphur such as furyl, pyridyl, 1,2-, 1,3- and 1,4-diazinyl, thienyl, isoxazolyl, oxazolyl, imidazolyl, or pyrrolyl. The term “heteroaryl” further refers to bicyclic aromatic groups comprising two 5- or 6-membered rings, in which one or both rings can contain 1, 2 or 3 atoms selected from nitrogen, oxygen or sulphur such as e.g. indole or quinoline, or partially hydrogenated bicyclic aromatic groups such as e.g. indolinyl. A heteroaryl group may have a substitution pattern as described earlier in connection with the term “aryl”. Preferred heteroaryl groups are e.g. thienyl and furyl which can optionally be substituted as described above, preferably with halogen, CF₃, lower-alkyl and/or lower-alkoxy.

The term “protecting group” refers to groups such as e.g. acyl, alkoxycarbonyl, aryloxycarbonyl, silyl, or imine-derivatives, which are used to temporarily block the reactivity of functional groups. Well known protecting groups are e.g. tert-butyloxycarbonyl, benzyloxycarbonyl, fluorenylmethyloxycarbonyl or diphenylmethylene which can be used for the protection of amino groups, or lower-alkyl-, β-trimethylsilylethyl- and β-trichloroethyl-esters, which can be used for the protection of carboxy groups.

“Isomers” are compounds that have identical molecular formulae but that differ in the nature or the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images are termed “enantiomers”, or sometimes optical isomers. A carbon atom bonded to four nonidentical substituents is termed a “chiral center”.

The term “pharmaceutically acceptable salts” embraces salts of the compounds of formula (I) with pharmaceutically acceptable bases such as alkali salts, e.g. Na- and K-salts, alkaline earth salts, e.g. Ca- and Mg-salts, and ammonium or substituted ammonium salts, such as e.g. trimethylammonium salts. The term “pharmaceutically acceptable salts” also relates to such salts.

The compounds of formula (I) can also be solvated, e.g. hydrated. The solvation can be effected in the course of the manufacturing process or can take place e.g. as a consequence of hygroscopic properties of an initially anhydrous compound of formula (I) (hydration). The term pharmaceutically acceptable salts also includes pharmaceutically acceptable solvates.

The term “pharmaceutically acceptable esters” embraces derivatives of the compounds of formula (I), in which a carboxy group has been converted to an ester. Lower-alkyl, hydroxy-lower-alkyl, lower-alkoxy-lower-alkyl, amino-lower-alkyl, mono- or di-lower-alkyl-amino-lower-alkyl, morpholino-lower-alkyl, pyrrolidino-lower-alkyl, piperidino-lower-alkyl, piperazino-lower-alkyl, lower-alkyl-piperazino-lower-alkyl and aralkyl esters are examples of suitable esters. The methyl, ethyl, propyl, butyl and benzyl esters are preferred esters. The methyl and ethyl esters are especially preferred. The term “pharmaceutically acceptable esters” furthermore embraces compounds of formula (I) in which hydroxy groups have been converted to the corresponding esters with inorganic or organic acids such as, nitric acid, sulphuric acid, phosphoric acid, citric acid, formic acid, maleic acid, acetic acid, succinic acid, tartaric acid, methanesulphonic acid, p-toluenesulphonic acid and the like, which are non toxic to living organisms.

In detail, the present invention relates to compounds of formula 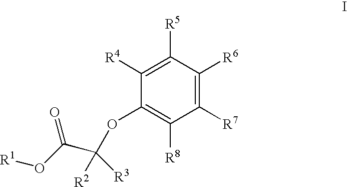
wherein
R¹ is hydrogen or C₁₋₇-alkyl;
R² and R³ are independently from each other hydrogen or C₁₋₇-alkyl,
R⁴ and R⁸ independently from each other are selected from the group consisting of hydrogen, C₁₋₇-alkyl, C₃₋₇-cycloalkyl, halogen, C₁₋₇-alkoxy-C₁₋₇-alkyl,
C₂₋₇-alkenyl, C₂₋₇-alkinyl, fluoro-C₁₋₇-alkyl, cyano-C₁₋₇-alkyl and cyano;
R⁵, R⁶ and R⁷ independently from each other are selected from the group consisting of hydrogen, C₁₋₇-alkyl, C₃₋₇-cycloalkyl, halogen, C₁₋₇-alkoxy-C₁₋₇-alkyl,
C₂₋₇-alkenyl, C₂₋₇-alkinyl, fluoro-C₁₋₇-alkyl, cyano-C₁₋₇-alkyl and cyano;
and one of R⁵, R⁶ and R⁷ is 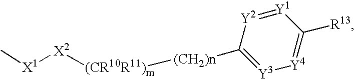
wherein
X¹ is selected from the group consisting of
—(CR¹⁴R¹⁵), —(CR¹⁴R¹⁵)CH₂—, —CH₂(CR¹⁴R¹⁵)—, —CH₂CH₂CH₂—,
—(CR¹⁴R¹⁵)CH₂CH₂—, —CH₂(CR¹⁴R¹⁵)CH₂—, —CH₂CH₂(CR¹⁴R¹⁵)—,
—CH₂CH₂CH₂CH₂—, —(CR¹⁴R¹⁵)CH₂CH₂CH₂—, —CH₂(CR¹⁴R¹⁵)CH₂CH₂—,
—CH₂CH₂(CR¹⁴R¹⁵)CH₂—, and —CH₂CH₂CH₂(CR¹⁴R¹⁵)—,
or, in addition,
X¹ is selected from the group consisting of
—OCH₂—, —O(CR¹⁴R¹⁵)—, —OCH₂CH₂—, —O(CR¹⁴H)CH₂—,
—OCH₂(CR¹⁴R¹⁵)—, —OCH₂CH₂CH₂—, —O(CR¹⁴H)CH₂CH₂—,
—OCH₂(CR¹⁴R¹⁵)CH₂—, and —OCH₂CH₂(CR¹⁴R¹⁵)—, when X² is —CONR⁹—; or
X¹ is selected from the group consisting of
—OCH₂CH₂—, —O(CR¹⁴H)CH₂—, —OCH₂(CR¹⁴R¹⁵)—,
—OCH₂CH₂CH₂—, —O(CR¹⁴H)CH₂CH₂—, —OCH₂(CR¹⁴R¹⁵)CH₂—, and
—OCH₂CH₂(CR¹⁴R¹⁵)—, when X² is —NR⁹CO—,
X² is —NR⁹CO— or —CONR⁹—;
R⁹ is selected from the group consisting of hydrogen, C₁₋₇-alkyl, C₃₋₇-cycloalkyl, fluoro-C₁₋₇-alkyl, hydroxy-C₂₋₇-alkyl, and C₁₋₇-alkoxy-C₂₋₇-alkyl;
Y¹, Y², Y³ and Y⁴ are N or C—R¹², whereas none, one or two of Y¹, Y², Y³ and Y⁴ are N and the other ones are C—R¹²;
R¹⁰ is selected from the group consisting of C₁₋₇-alkyl, C₃₋₇-cycloalkyl, fluoro-C₁₋₇-alkyl, and C₁₋₇-alkoxy-C₁₋₇-alkyl;
R¹¹ is selected from the group consisting of hydrogen, C₁₋₇-alkyl, and
C₁₋₇-alkoxy-C₁₋₇-alkyl;
R¹² independently from each other in each occurrence is selected from the group consisting of hydrogen, C₁₋₇-alkyl, C₃₋₇-cycloalkyl, fluoro-C₁₋₇-alkyl, C₁₋₇-alkoxy-C₁₋₇-alkyl, hydroxy-C₁₋₇-alkyl, C₁₋₇-alkylthio-C₁₋₇-alkyl, carboxy-C₁₋₇-alkoxy-C₁₋₇-alkyl, carboxy, carboxy-C₁₋₇-alkyl, mono- or di-C₁₋₇-alkyl-amino-C₁₋₇-alkyl,
C₁₋₇-alkanoyl-C₁₋₇-alkyl, C₂₋₇-alkenyl, and C₂₋₇-alkinyl;
R¹³ is aryl or heteroaryl;
R¹⁴ is selected from the group consisting of C₁₋₇-alkyl, C₃₋₇-cycloalkyl, fluoro-C₁₋₇-alkyl, and C₁₋₇-alkoxy-C₁₋₇-alkyl;
R¹⁵ is selected from the group consisting of hydrogen, C₁₋₇-alkyl, C₃₋₇-cycloalkyl, fluoro-C₁₋₇-alkyl, and C₁₋₇-alkoxy-C₁₋₇-alkyl;
m is 0 or 1;
n is 0, 1, 2 or 3, and
all pharmaceutically acceptable salts and/or esters thereof.

Preferred compounds of the present invention are for example those, wherein one or two of Y¹, Y², Y³ and Y⁴ are N and the other ones are C—R¹². Included in this group are for example compounds, wherein one of Y¹, Y², Y³ and Y⁴ is N and the other ones are C—R¹², thus meaning compounds containing a pyridyl group.

Especially preferred are those compounds of formula I, wherein Y¹ is N and Y², Y³ and Y⁴ are C—R¹², e.g. compounds of formula I containing the group

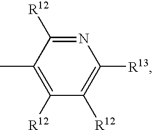

Further preferred compounds of the present invention are those, wherein two of Y¹, Y², Y³ and Y⁴ are N and the other ones are C—R¹², thus meaning compounds containing a pyrazinyl group or a pyrimidinyl group or a pyridazinyl group.

Especially preferred are compounds of formula I, wherein Y¹ and Y⁴ are N and Y² and Y³ are C—R¹², e.g. compounds of formula I containing the pyrimidinyl group

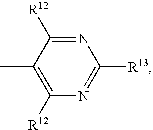

Also preferred are compounds of formula I, wherein Y¹ and Y³ are N and Y² and Y are C—R¹², e.g. compounds of formula I containing the pyrazinyl group 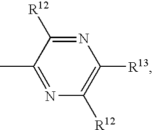
R¹² is preferably hydrogen, C₁₋₇-alkyl, C₃₋₇-cycloalkyl, fluoro-C₁₋₇-alkyl, or C₁₋₇-alkoxy-C₁₋₇-alkyl.

Further preferred compounds of formula I of the present invention are those, wherein X² is —NR⁹CO—;

X¹ is selected from the group consisting of

—(CR¹⁴R¹⁵), —(CR¹⁴R¹⁵)CH₂—, —CH₂(CR¹⁴R¹⁵)—, —CH₂CH₂CH₂—,

—(CR¹⁴R¹⁵)CH₂CH₂—, —CH₂(CR¹⁴R¹⁵)CH₂—, —CH₂CH₂(CR¹⁴R¹⁵)—,

—CH₂CH₂CH₂CH₂—, —(CR¹⁴R¹⁵)CH₂CH₂CH₂—, —CH₂(CR¹⁴R¹⁵)CH₂CH₂—,

—CH₂CH₂(CR¹⁴R¹⁵)CH₂—, —CH₂CH₂CH₂(CR¹⁴R¹⁵)—,

—OCH₂CH₂—, —O(CR¹⁴H)CH₂—, —OCH₂(CR¹⁴R¹⁵)—,

—OCH₂CH₂CH₂—, —O(CR¹⁴H)CH₂CH₂—, —OCH₂(CR¹⁴R¹⁵)CH₂—, and

—OCH₂CH₂(CR¹⁴R¹⁵)—;

R⁹ is selected from the group consisting of hydrogen, C₁₋₇-alkyl, C₃₋₇-cycloalkyl, fluoro-C₁₋₇-alkyl, hydroxy-C₂₋₇-alkyl, and C₁₋₇-alkoxy-C₂₋₇-alkyl;

R¹⁴ is selected from the group consisting of C₁₋₇-alkyl, C₃₋₇-cycloalkyl, fluoro-C₁₋₇-alkyl, and C₁₋₇-alkoxy-C₁₋₇-alkyl; and

R¹⁵ is selected from the group consisting of hydrogen, C₁₋₇-alkyl, C₃₋₇-cycloalkyl, fluoro-C₁₋₇-alkyl, and C₁₋₇-alkoxy-C₁₋₇-alkyl.

Within this group, those compounds are more preferred, wherein R¹⁴ is C₁₋₇-alkyl, preferably methyl or ethyl, and R¹⁵ is hydrogen. Especially preferred are those compounds of formula I, wherein X¹ is selected from the group consisting of —CH(CH₃)—, —CH(C₂H₅)—, —CH₂—CH(CH₃)—, —OCH₂CH₂— and —O—(CHCH₃)—CH₂—.

Another group of preferred compounds of formula I are those, wherein

X² is —CONR⁹—;

X¹ is selected from the group consisting of

—(CR¹⁴R¹⁵), —(CR¹⁴R¹⁵)CH₂—, —CH₂(CR¹⁴R¹⁵)—, —CH₂CH₂CH₂—,

—(CR¹⁴R¹⁵)CH₂CH₂—, —CH₂(CR¹⁴R¹⁵)CH₂—, —CH₂CH₂(CR¹⁴R¹⁵)—,

—CH₂CH₂CH₂CH₂—, —(CR¹⁴R¹⁵)CH₂CH₂CH₂—, —CH₂(CR¹⁴R¹⁵)CH₂CH₂—,

—CH₂CH₂(CR¹⁴R¹⁵)CH₂—, —CH₂CH₂CH₂(CR¹⁴R¹⁵)—,

—OCH₂—, —O(CR¹⁴R¹⁵)—, —OCH₂CH₂—, —O(CR¹⁴H)CH₂—,

—OCH₂(CR¹⁴R¹⁵)—, —OCH₂CH₂CH₂—, —O(CR¹⁴H)CH₂CH₂—,

—OCH₂(CR¹⁴R¹⁵)CH₂—, and —OCH₂CH₂(CR¹⁴R¹⁵)—;

R⁹ is selected from the group consisting of hydrogen, C₁₋₇-alkyl, C₃₋₇-cycloalkyl, fluoro-C₁₋₇-alkyl, hydroxy-C₂₋₇-alkyl, and C₁₋₇-alkoxy-C₂₋₇-alkyl;

R¹⁴ is selected from the group consisting of C₁₋₇-alkyl, C₃₋₇-cycloalkyl, fluoro-C₁₋₇-alkyl, and C₁₋₇-alkoxy-C₁₋₇-alkyl; and

R¹⁵ is selected from the group consisting of hydrogen, C₁₋₇-alkyl, C₃₋₇-cycloalkyl, fluoro-C₁₋₇-alkyl, and C₁₋₇-alkoxy-C₁₋₇-alkyl.

Within this group, those compounds are more preferred, wherein R¹⁴ is C₁₋₇-alkyl, preferably methyl or ethyl, and R¹⁵ is hydrogen. Especially preferred are those compounds of formula I, wherein X² is —CONR⁹— and X¹ is selected from the group consisting of —CH(CH₃)—, —CH₂CH₂CH₂—, —CH(CH₃)CH₂—, —OCH₂—, and —OCH(CH₃)—.

Especially preferred are compounds of formula I of the present invention, wherein R⁹ is hydrogen.

Also preferred are compounds of formula I according to the invention, wherein

X¹ is selected from the group consisting of

—(CR¹⁴R¹⁵), —(CR¹⁴R¹⁵)CH₂—, —CH₂(CR¹⁴R¹⁵)—, —CH₂CH₂CH₂—,

—(CR¹⁴R¹⁵)CH₂CH₂—, —CH₂(CR¹⁴R¹⁵)CH₂—, —CH₂CH₂(CR¹⁴R¹⁵)—,

—CH₂CH₂CH₂CH₂—, —(CR¹⁴R¹⁵)CH₂CH₂CH₂—, —CH₂(CR¹⁴R¹⁵)CH₂CH₂—,

—CH₂CH₂(CR¹⁴R¹⁵)CH₂—, and —CH₂CH₂CH₂(CR¹⁴R¹⁵)—;

R¹⁴ is C₁₋₇-alkyl and R¹⁵ is hydrogen.

Another group of preferred compounds of the present invention are those, wherein Y¹, Y², Y³ and Y⁴ are C—R¹².

R¹² is preferably independently selected from the group consisting of hydrogen, C₁₋₇-alkyl, C₃₋₇-cycloalkyl, fluoro-C₁₋₇-alkyl, or C₁₋₇-alkoxy-C₁₋₇-alkyl. Most preferred are those compounds, wherein R¹² is hydrogen.

Preferred are furthermore compounds of formula I of the present invention, wherein R⁶ is 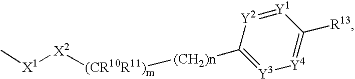
and R⁴, R⁵, R⁷ and R⁸ independently from each other are selected from hydrogen or C₁₋₇-alkyl.

These compounds have the formula I-A:

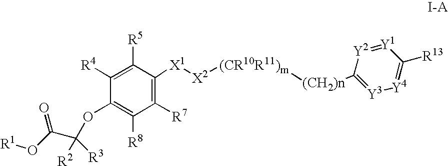

Also preferred are compounds of formula I according to the invention, wherein R⁵ or R⁷ is

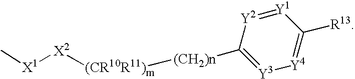

These compounds have the formula I-B or I-C:

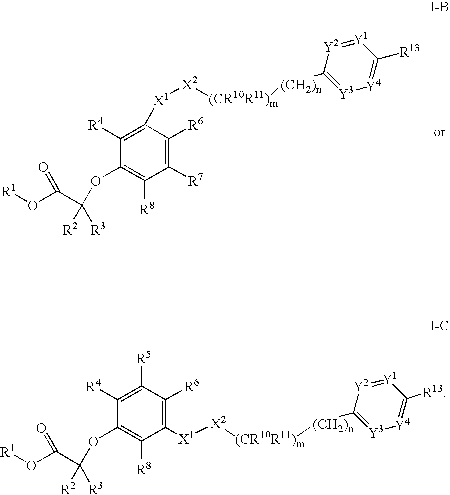

Furthermore, compounds of formula I, wherein R¹ is hydrogen, are preferred.

Compounds of formula I, wherein R² and R³ independently from each other are hydrogen or methyl, are also preferred. Also preferred are compounds of formula I, wherein at least one of R² and R³ is methyl. Even more preferred are compounds of formula I, wherein R² and R³ are methyl.

The integer m is 0 or 1. Preferred are compounds of formula I, wherein m is 0.

The integer n is 0, 1, 2 or 3. Preferred are compounds of formula I, wherein n is 0. However, compounds of formula I, wherein n is 1 are also preferred.

Compounds of formula I, wherein R¹³ is aryl, are preferred. More preferred are those compounds of formula I, wherein R¹³ is unsubstituted phenyl or phenyl substituted with one to three groups selected from C₁₋₇-alkyl, C₁₋₇-alkoxy, halogen, fluoro-C₁₋₇-alkyl, fluoro-C₁₋₇-alkoxy and cyano, with those compounds, wherein R¹³ is phenyl substituted with halogen, fluoro-C₁₋₇-alkyl or fluoro-C₁₋₇-alkoxy, being particularly preferred.

Examples of preferred compounds of formula I are the following:
[rac]-2-[4-(1-{[4-cyclopropyl-2-(4-trifluoromethyl-phenyl)-pyrimidine-5-carbonyl]-amino}-ethyl)-2-methyl-phenoxy]-2-methyl-propionic acid,
[rac]-2-[4-(1-{[4-cyclopropyl-2-(3-trifluoromethyl-phenyl)-pyrimidine-5-carbonyl]-amino}-ethyl)-2-methyl-phenoxy]-2-methyl-propionic acid,
[rac]-2-methyl-2-(2-methyl-4-{1-[(3′-trifluoromethyl-biphenyl-4-carbonyl)-amino]-ethyl}-phenoxy)-propionic acid,
[rac]-2-methyl-2-(2-methyl-4-{1-[(4′-trifluoromethyl-biphenyl-4-carbonyl)-amino]-ethyl}-phenoxy)-propionic acid,
[rac]-2-methyl-2-(2-methyl-4-{1-[2-methyl-6-(4-trifluoromethyl-phenyl)-pyridin-3-ylcarbamoyl]-ethoxy}-phenoxy)-propionic acid,
[rac]-2-{4-[1-(biphenyl-4-ylcarbamoyl)-ethoxy]-2-methyl-phenoxy}-2-methyl-propionic acid,
[rac]-2-(4-{1-[4-cyclopropyl-2-(4-trifluoromethyl-phenyl)-pyrimidin-5-ylcarbamoyl]-ethoxy}-2-methyl-phenoxy)-2-methyl-propionic acid,
[rac]-2-methyl-2-{2-methyl-4-[1-(3′-trifluoromethyl-biphenyl-4-ylcarbamoyl)-ethoxy]-phenoxy}-propionic acid,
[rac]-2-methyl-2-{2-methyl-4-[1-(4′-trifluoromethyl-biphenyl-3-ylcarbamoyl)-ethoxy]-phenoxy}-propionic acid,
2-methyl-2-(2-methyl-4-{[2-methyl-6-(4-trifluoromethyl-phenyl)-pyridin-3-ylcarbamoyl]-methoxy}-phenoxy)-propionic acid,
2-[4-(biphenyl-4-ylcarbamoylmethoxy)-2-methyl-phenoxy]-2-methyl-propionic acid,
2-(4-{[4-cyclopropyl-2-(4-trifluoromethyl-phenyl)-pyrimidin-5-ylcarbamoyl]-methoxy}-2-methyl-phenoxy)-2-methyl-propionic acid,
2-methyl-2-{2-methyl-4-[(3′-trifluoromethyl-biphenyl-4-ylcarbamoyl)-methoxy]-phenoxy}-propionic acid,
2-methyl-2-{2-methyl-4-[(4′-trifluoromethyl-biphenyl-3-ylcarbamoyl)-methoxy]-phenoxy}-propionic acid,
2-methyl-2-(4-{3-[2-methyl-6-(4-trifluoromethyl-phenyl)-pyridin-3-ylcarbamoyl]-propyl}-phenoxy)-propionic acid,
2-(4-{3-[4-cyclopropyl-2-(4-trifluoromethyl-phenyl)-pyrimidin-5-ylcarbamoyl]-propyl}-phenoxy)-2-methyl-propionic acid,
2-methyl-2-{4-[3-(3′-trifluoromethyl-biphenyl-4-ylcarbamoyl)-propyl]-phenoxy}-propionic acid,
2-methyl-2-{4-[3-(4′-trifluoromethyl-biphenyl-3-ylcarbamoyl)-propyl]-phenoxy}-propionic acid,
2-methyl-2-[2-methyl-4-(2-{[2-methyl-6-(4-trifluoromethyl-phenyl)-pyridine-3-carbonyl]-amino}-ethoxy)-phenoxy]-propionic acid,
2-[4-(2-{[4-cyclopropyl-2-(4-trifluoromethyl-phenyl)-pyrimidine-5-carbonyl]-amino}-ethoxy)-2-methyl-phenoxy]-2-methyl-propionic acid,
2-methyl-2-[2-methyl-4-(2-{[4-trifluoromethyl-2-(4-trifluoromethyl-phenyl)-pyrimidine-5-carbonyl]-amino}-ethoxy)-phenoxy]-propionic acid,
2-[4-(2-{[4-methoxymethyl-2-(4-trifluoromethyl-phenyl)-pyrimidine-5-carbonyl]-amino}-ethoxy)-2-methyl-phenoxy]-2-methyl-propionic acid,
2-[4-(2-{2-[4-cyclopropyl-2-(4-trifluoromethyl-phenyl)-pyrimidin-5-yl]-acetylamino}-ethoxy)-2-methyl-phenoxy]-2-methyl-propionic acid,
2-methyl-2-(2-methyl-4-{2-[(4′-trifluoromethyl-biphenyl-4-carbonyl)-amino]-ethoxy}-phenoxy)-propionic acid,
2-methyl-2-(2-methyl-4-{2-[(3′-trifluoromethyl-biphenyl-4-carbonyl)-amino]-ethoxy}-phenoxy)-propionic acid,
2-[4-(2-{[4-cyclopropyl-2-(3-trifluoromethyl-phenyl)-pyrimidine-5-carbonyl]-amino}-ethoxy)-2-methyl-phenoxy]-2-methyl-propionic acid,
[rac]-2-[4-(2-{[4-cyclopropyl-2-(4-trifluoromethyl-phenyl)-pyrimidine-5-carbonyl]-amino}-1-methyl-ethoxy)-2-methyl-phenoxy]-2-methyl-propionic acid,
[rac]-2-[4-(2-{[4-cyclopropyl-2-(3-trifluoromethyl-phenyl)-pyrimidine-5-carbonyl]-amino}-1-methyl-ethoxy)-2-methyl-phenoxy]-2-methyl-propionic acid,
[rac]-2-methyl-2-(2-methyl-4-{1-methyl-2-[(3′-trifluoromethyl-biphenyl-4-carbonyl)-amino]-ethoxy}-phenoxy)-propionic acid,
[rac]-2-methyl-2-(2-methyl-4-{1-methyl-2-[(4′-trifluoromethyl-biphenyl-4-carbonyl)-amino]-ethoxy}-phenoxy)-propionic acid,
[rac]-2-[4-(2-{[4-cyclopropyl-2-(4-trifluoromethyl-phenyl)-pyrimidine-5-carbonyl]-amino}-propyl)-2-methyl-phenoxy]-2-methyl-propionic acid,
[rac]-2-[4-(2-{[4-cyclopropyl-2-(3-trifluoromethyl-phenyl)-pyrimidine-5-carbonyl]-amino}-propyl)-2-methyl-phenoxy]-2-methyl-propionic acid,
[rac]-2-methyl-2-(2-methyl-4-{2-[(3′-trifluoromethyl-biphenyl-4-carbonyl)-amino]-propyl}-phenoxy)-propionic acid,
[rac]-2-methyl-2-(2-methyl-4-{2-[(4′-trifluoromethyl-biphenyl-4-carbonyl)-amino]-propyl}-phenoxy)-propionic acid,
[rac]-2-[4-(1-{[4-cyclopropyl-2-(4-trifluoromethyl-phenyl)-pyrimidine-5-carbonyl]-amino}-propyl)-2-methyl-phenoxy]-2-methyl-propionic acid,
[rac]-2-[4-(1-{[4-cyclopropyl-2-(3-trifluoromethyl-phenyl)-pyrimidine-5-carbonyl]-amino}-propyl)-2-methyl-phenoxy]-2-methyl-propionic acid,
[rac]-2-methyl-2-(2-methyl-4-{1-[(3′-trifluoromethyl-biphenyl-4-carbonyl)-amino]-propyl}-phenoxy)-propionic acid,
[rac]-2-methyl-2-(2-methyl-4-{1-[(4′-trifluoromethyl-biphenyl-4-carbonyl)-amino]-propyl}-phenoxy)-propionic acid,
[rac]-2-methyl-2-(2-methyl-4-{1-methyl-2-[2-methyl-6-(4-trifluoromethyl-phenyl)-pyridin-3-ylcarbamoyl]-ethyl}-phenoxy)-propionic acid,
[rac]-2-(4-{2-[4-cyclopropyl-2-(4-trifluoromethyl-phenyl)-pyrimidin-5-ylcarbamoyl]-1-methyl-ethyl}-2-methyl-phenoxy)-2-methyl-propionic acid,
[rac]-2-methyl-2-{2-methyl-4-[1-methyl-2-(4′-trifluoromethyl-biphenyl-4-ylcarbamoyl)-ethyl]-phenoxy}-propionic acid,
[rac]-2-methyl-2-{2-methyl-4-[1-methyl-2-(3′-trifluoromethyl-biphenyl-4-ylcarbamoyl)-ethyl]-phenoxy}-propionic acid,
[rac]-2-(4-{1-[4-cyclopropyl-2-(4-trifluoromethyl-phenyl)-pyrimidin-5-ylcarbamoyl]-ethyl}-2-methyl-phenoxy)-2-methyl-propionic acid,
[rac]-2-methyl-2-(2-methyl-4-{1-[2-methyl-6-(4-trifluoromethyl-phenyl)-pyridin-3-ylcarbamoyl]-ethyl}-phenoxy)-propionic acid,
[rac]-2-methyl-2-{2-methyl-4-[1-(4′-trifluoromethyl-biphenyl-4-ylcarbamoyl)-ethyl]-phenoxy}-propionic acid,
[rac]-2-methyl-2-{2-methyl-4-[1-(3′-trifluoromethyl-biphenyl-4-ylcarbamoyl)-ethyl]-phenoxy}-propionic acid, and
[rac]-2-methyl-2-[2-methyl-4-(1-{[2-(4-trifluoromethoxy-phenyl)-4-trifluoromethyl-pyrimidine-5-carbonyl]-amino}-ethyl)-phenoxy]-propionic acid.

Particularly preferred compounds of formula I of the present invention are the following:
[rac]-2-[4-(1-{[4-cyclopropyl-2-(4-trifluoromethyl-phenyl)-pyrimidine-5-carbonyl]-amino}-ethyl)-2-methyl-phenoxy]-2-methyl-propionic acid,
[rac]-2-methyl-2-(2-methyl-4-{[1-[(3′-trifluoromethyl-biphenyl-4-carbonyl)-amino]-ethyl}-phenoxy)-propionic acid,
[rac]-2-(4-{1-[4-cyclopropyl-2-(4-trifluoromethyl-phenyl)-pyrimidin-5-ylcarbamoyl]-ethoxy}-2-methyl-phenoxy)-2-methyl-propionic acid,
2-methyl-2-{2-methyl-4-[(3′-trifluoromethyl-biphenyl-4-ylcarbamoyl)-methoxy]-phenoxy}-propionic acid,
2-(4-{3-[4-cyclopropyl-2-(4-trifluoromethyl-phenyl)-pyrimidin-5-ylcarbamoyl]-propyl}-phenoxy)-2-methyl-propionic acid,
2-[4-(2-{[4-cyclopropyl-2-(3-trifluoromethyl-phenyl)-pyrimidine-5-carbonyl]-amino}-ethoxy)-2-methyl-phenoxy]-2-methyl-propionic acid,
[rac]-2-[4-(2-{4-cyclopropyl-2-(4-trifluoromethyl-phenyl)-pyrimidine-5-carbonyl]-amino}-propyl)-2-methyl-phenoxy]-2-methyl-propionic acid,
[rac]-2-methyl-2-(2-methyl-4-{1-[(4′-trifluoromethyl-biphenyl-4-carbonyl)-amino]-propyl}-phenoxy)-propionic acid,
[rac]-2-methyl-2-(2-methyl-4-{1-methyl-2-[2-methyl-6-(4-trifluoromethyl-phenyl)-pyridin-3-ylcarbamoyl]-ethyl}-phenoxy)-propionic acid, and
[rac]-2-methyl-2-{2-methyl-4-[1-(3′-trifluoromethyl-biphenyl-4-ylcarbamoyl)-ethyl]-phenoxy}-propionic acid.

Especially preferred are also the following compounds of formula I of the present invention:
[rac]-2-[4-(1-{[4-cyclopropyl-2-(4-trifluoromethyl-phenyl)-pyrimidine-5-carbonyl]-amino}-ethyl)-2-methyl-phenoxy]-2-methyl-propionic acid,
[rac]-2-(4-{1-[4-cyclopropyl-2-(4-trifluoromethyl-phenyl)-pyrimidin-5-ylcarbamoyl]-ethoxy}-2-methyl-phenoxy)-2-methyl-propionic acid,
2-(4-{3-[4-cyclopropyl-2-(4-trifluoromethyl-phenyl)-pyrimidin-5-ylcarbamoyl]-propyl}-phenoxy)-2-methyl-propionic acid,
2-[4-(2-{[4-cyclopropyl-2-(3-trifluoromethyl-phenyl)-pyrimidine-5-carbonyl]-amino}-ethoxy)-2-methyl-phenoxy]-2-methyl-propionic acid, and
[rac]-2-[4-(2-{[4-cyclopropyl-2-(4-trifluoromethyl-phenyl)-pyrimidine-5-carbonyl]-amino}-propyl)-2-methyl-phenoxy]-2-methyl-propionic acid.

Furthermore, the pharmaceutically acceptable salts of the compounds of formula I and the pharmaceutically acceptable esters of the compounds of formula I individually constitute preferred embodiments of the present invention.

Compounds of formula I can have one or more asymmetric carbon atoms and can exist in the form of optically pure enantiomers, mixtures of enantiomers such as, for example, racemates, optically pure diastereoisomers, mixtures of diastereoisomers, diastereoisomeric racemates or mixtures of diastereoisomeric racemates. The optically active forms can be obtained for example by resolution of the racemates, by asymmetric synthesis or asymmetric chromatography (chromatography with a chiral adsorbens or eluant). The invention embraces all of these forms.

It will be appreciated, that the compounds of general formula I in this invention may be derivatized at functional groups to provide derivatives which are capable of conversion back to the parent compound in vivo. Physiologically acceptable and metabolically labile derivatives, which are capable of producing the parent compounds of general formula I in vivo are also within the scope of this invention.

A further aspect of the present invention is the process for the manufacture of compounds of formula I as defined above, which process comprises reacting a compound of formula 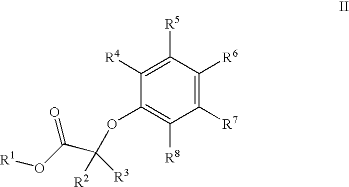
wherein R¹ is C₁₋₇-alkyl, R² to R⁸ are as defined above and one of R⁵, R⁶ or R⁷ is —X¹—COOH,
with a compound of formula 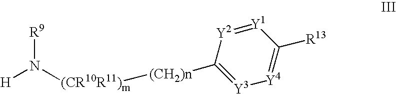
wherein Y¹ to Y⁴, R⁹, R¹⁰, R¹¹, R¹³, m and n are as defined above,
to obtain a compound of formula 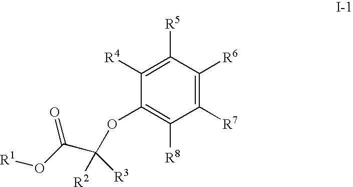
wherein one of R⁵, R⁶ and R⁷ is 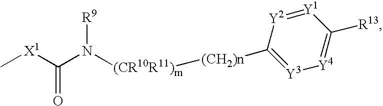
and wherein R¹ is C₁₋₇-alkyl and X¹, Y¹ to Y⁴, R² to R¹³ and m and n are as defined above, and optionally hydrolyzing the ester group to obtain a compound of formula I-1, wherein R¹ is hydrogen;
or, alternatively,
reacting a compound of formula 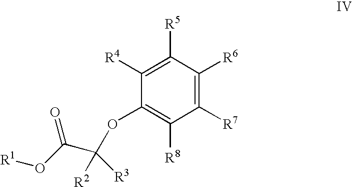
wherein R¹ is C₁₋₇-alkyl, R² to R⁸ are as defined above and one of R⁵, R⁶ or R⁷ is —X¹—NHR⁹, wherein X¹ and R⁹ are as defined above,
with a compound of formula 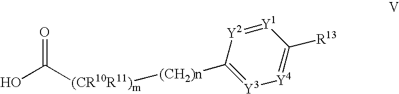
wherein Y¹ to Y⁴, R¹⁰, R¹¹, R¹³, m and n are as defined above,
to obtain a compound of formula 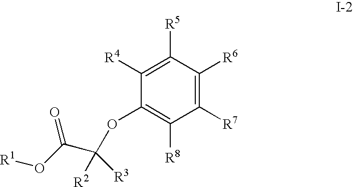
wherein one of R⁵, R⁶ and R⁷ is 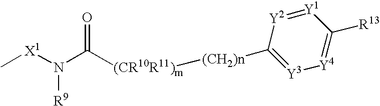
and wherein R¹ is C₁₋₇-alkyl and X¹, Y¹ to Y⁴, R² to R¹³ and m and n are as defined above, and optionally hydrolyzing the ester group to obtain a compound of formula I-2, wherein R¹ is hydrogen.

As described above, the compounds of formula (I) of the present invention can be used as medicaments for the treatment and/or prevention of diseases which are modulated by PPARδ and/or PPARα agonists. Examples of such diseases are diabetes, particularly non-insulin dependent diabetes mellitus, increased lipid and cholesterol levels, particularly low HDL-cholesterol, high LDL-cholesterol, or high triglyceride levels, atherosclerotic diseases, metabolic syndrome, syndrome X, obesity, elevated blood pressure, endothelial dysfunction, procoagulant state, dyslipidemia, polycystic ovary syndrome, inflammatory diseases (such as e.g. Crohn's disease, inflammatory bowel disease, colitis, pancreatitis, cholestasis/fibrosis of the liver, rheumatoid arthritis, osteoarthritis, psoriasis and other skin disorders, and diseases that have an inflammatory component such as e.g. Alzheimer's disease or impaired/improvable cognitive function) and proliferative diseases (cancers such as e.g. liposarcoma, colon cancer, prostate cancer, pancreatic cancer and breast cancer). The use as medicament for the treatment of low HDL cholesterol levels, high LDL cholesterol levels, high triglyceride levels, metabolic syndrome and syndrome X is preferred.

The invention therefore also relates to pharmaceutical compositions comprising a compound as defined above and a pharmaceutically acceptable carrier and/or adjuvant.

Further, the invention relates to compounds as defined above for use as therapeutically active substances, particularly as therapeutic active substances for the treatment and/or prevention of diseases which are modulated by PPARδ and/or PPARα agonists. Examples of such diseases are diabetes, particularly non-insulin dependent diabetes mellitus, increased lipid and cholesterol levels, particularly low HDL-cholesterol, high LDL-cholesterol, or high triglyceride levels, atherosclerotic diseases, metabolic syndrome, syndrome X, obesity, elevated blood pressure, endothelial dysfunction, procoagulant state, dyslipidemia, polycystic ovary syndrome, inflammatory diseases such as rheumatoid arthritis, osteoarthritis, psoriasis and other skin disorder, and proliferative diseases.

In another embodiment, the invention relates to a method for the treatment and/or prevention of diseases which are modulated by PPARδ and/or PPARα agonists, which method comprises administering a compound of formula (I) to a human or animal. Preferred examples of such diseases are diabetes, particularly non-insulin dependent diabetes mellitus, increased lipid and cholesterol levels, particularly low HDL-cholesterol, high LDL-cholesterol, or high triglyceride levels, atherosclerotic diseases, metabolic syndrome, syndrome X, obesity, elevated blood pressure, endothelial dysfunction, procoagulant state, dyslipidemia, polycystic ovary syndrome, inflammatory diseases such as rheumatoid arthritis, osteoarthritis, psoriasis and other skin disorder, and proliferative diseases.

The invention further relates to the use of compounds as defined above for the treatment and/or prevention of diseases which are modulated by PPARδ and/or PPARα agonists. Preferred examples of such diseases are diabetes, particularly non-insulin dependent diabetes mellitus, increased lipid and cholesterol levels, particularly low HDL-cholesterol, high LDL-cholesterol, or high triglyceride levels, atherosclerotic diseases, metabolic syndrome, syndrome X, obesity, elevated blood pressure, endothelial dysfunction, procoagulant state, dyslipidemia, polycystic ovary syndrome, inflammatory diseases such as rheumatoid arthritis, osteoarthritis, psoriasis and other skin disorder, and proliferative diseases.

In addition, the invention relates to the use of compounds as defined above for the preparation of medicaments for the treatment and/or prevention of diseases which are modulated by PPARδ and/or PPARα agonists. Preferred examples of such diseases are diabetes, particularly non-insulin dependent diabetes mellitus, increased lipid and cholesterol levels, particularly low HDL-cholesterol, high LDL-cholesterol, or high triglyceride levels, atherosclerotic diseases, metabolic syndrome, syndrome X, obesity, elevated blood pressure, endothelial dysfunction, procoagulant state, dyslipidemia, polycystic ovary syndrome, inflammatory diseases such as rheumatoid arthritis, osteoarthritis, psoriasis and other skin disorder, and proliferative diseases. Such medicaments comprise a compound as defined above.

The compounds of formula I can be manufactured by the methods given below, by the methods given in the examples or by analogous methods. Appropriate reaction conditions for the individual reaction steps are known to a person skilled in the art. Starting materials are either commercially available or can be prepared by methods analogous to the methods given below, by methods described in references cited in the text or in the examples, or by methods known in the art.

The synthesis of compounds with the general structure I, particularly compounds according to formula Ia to Ih, are described in scheme 1 to scheme 3. Scheme 4 describes the synthesis of intermediates not covered by schemes 1, 2 and 3. Scheme 5 to scheme 8 describe the synthesis of synthons 10 and 11 (scheme 1), of synthon 10 (scheme 2) and of synthon 10 (scheme 3).

The synthesis of compounds with the general structure I, particularly compounds according to formula Ia and Ib with X¹ beginning with an oxygen atom can be accomplished according to scheme 1. Substituents R, R′ correspond to substituents as defined in detail in the claims.

Hydroxy aldehydes or hydroxy aryl alkyl ketones 1 are known or can be prepared by methods known in the art. Reaction of phenols 1 with alpha halo esters of formula 2 in the presence of a base like potassium or cesium carbonate in solvents like acetone, methyl-ethyl ketone, acetonitrile or N,N-dimethylformamide in a temperature range between room temperature and 140° C. leads to the corresponding ether compounds 3 (step a). Baeyer Villiger oxidation e.g. with meta chloro perbenzoic acid in a solvent like dichloromethane, leads to compounds 4 (step b). Phenols 4 can react with protected amino alcohols 5, e.g. via Mitsunobu-reaction, with triphenylphosphine and di-tert-butyl-, diisopropyl- or diethyl-azodicarboxylate as reagents; this transformation is preferably carried out in a solvent like toluene, dichloromethane or tetrahydrofurane at ambient temperature followed by optional N-alkylation (e.g. sodium hydride and a reactive alkyl halogenide/mesylate or triflate in a solvent like N,N-dimethylformamide) and deprotection (e.g. TFA/CH₂Cl₂, or HCl in dioxane at 0° C. to RT) leading to amino compounds 8 (steps c and d). Alternatively, phenols 4 can react with synthons 6 or 7, if a free hydroxy group is present e.g. via Mitsunobu-reaction; alternatively, if they carry a halide, mesylate, tosylate or triflate moiety, the synthons 6 or 7 can be reacted with phenols 4 in solvents like N,N-dimethylformamide, dimethylsulfoxide, acetonitrile, acetone or methyl-ethyl ketone in the presence of a weak base like cesium or potassium carbonate at a temperature ranging from room temperature to 140° C., preferably around 50° C. to yield the corresponding protected ether compounds (step e).

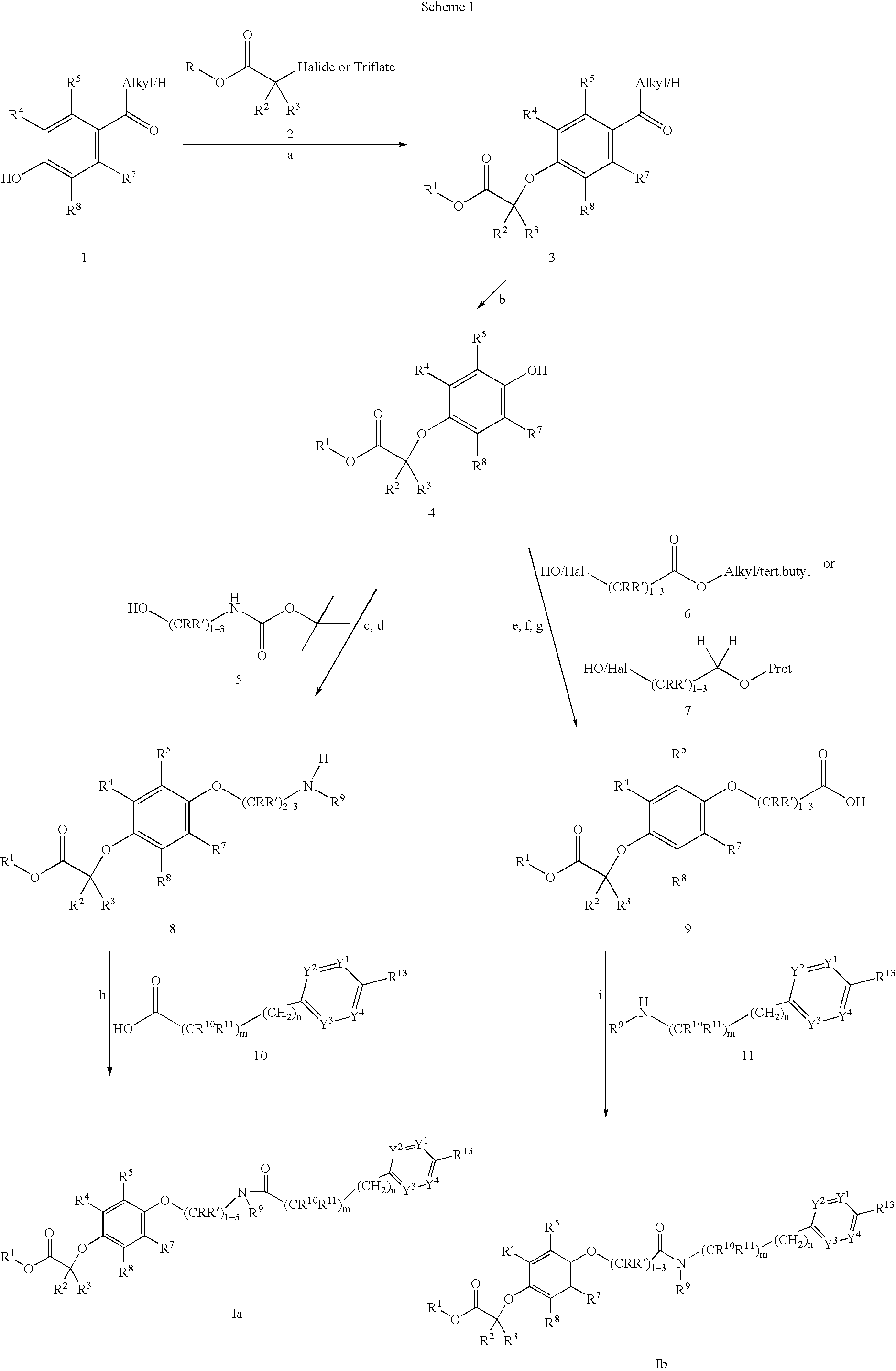

Depending on the synthon used, standard deprotection, or standard deprotection followed by oxidation yield acids 9 (step f, g) (e.g. Swern oxidation to the aldehyde: oxalyl chloride/dimethylsulfoxide/triethylamine in dichloromethane, −78° C. to room temperature; followed by oxidation to the acid with sodium chlorite, sodium dihydrogenphosphate-dihydrate in tert-butanol/water 2:1 in the presence of 2-methyl-2-butene at room temperature). Amines 8 or acids 9 can be chiral and can optionally be separated into optically pure antipodes by methods well known in the art, e.g. by chromatography on a chiral HPLC column. Condensation of amines 8 or acids 9 with acids 10 or amines 11 can be performed using well known procedures for amide formation, such as use of N-(3-dimethylaminopropyl)-N′-ethyl-carbodiimide-hydrochloride and optionally 4-dimethylamino-pyridine in dichloromethane at temperatures between 0° C. and room temperature yielding compounds Ia (step h) or Ib (step i). Those can optionally be hydrolyzed according to standard procedures, e.g. by treatment with an alkali hydroxide like LiOH or NaOH in a polar solvent mixture like tetrahydrofurane/ethanol/water to give carboxylic acids Ia or Ib. In case R¹ is equal to tert-butyl, treatment with e.g. trifluoroacetic acid, anisole in a solvent like dichloromethane between room temperature and the reflux temperature of the solvents yields carboxylic acids Ia or Ib.

An analogous reaction scheme with the same reaction sequences applies for the isomeric compound series leading to compounds of general formula I, particularly compounds according to formula Ic or Id.

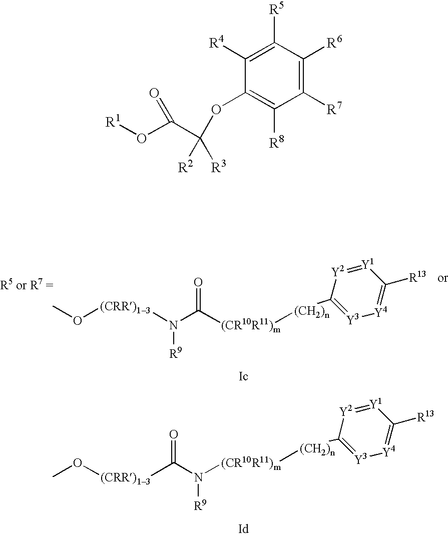

The synthesis of compounds with the general structure I, particularly compounds according to formula Ie with the X¹ substituent beginning with a carbon atom and X² equal to —CONR⁹— can be accomplished according to scheme 2. Substituents R, R′ correspond to substituents as defined in detail in the claims.

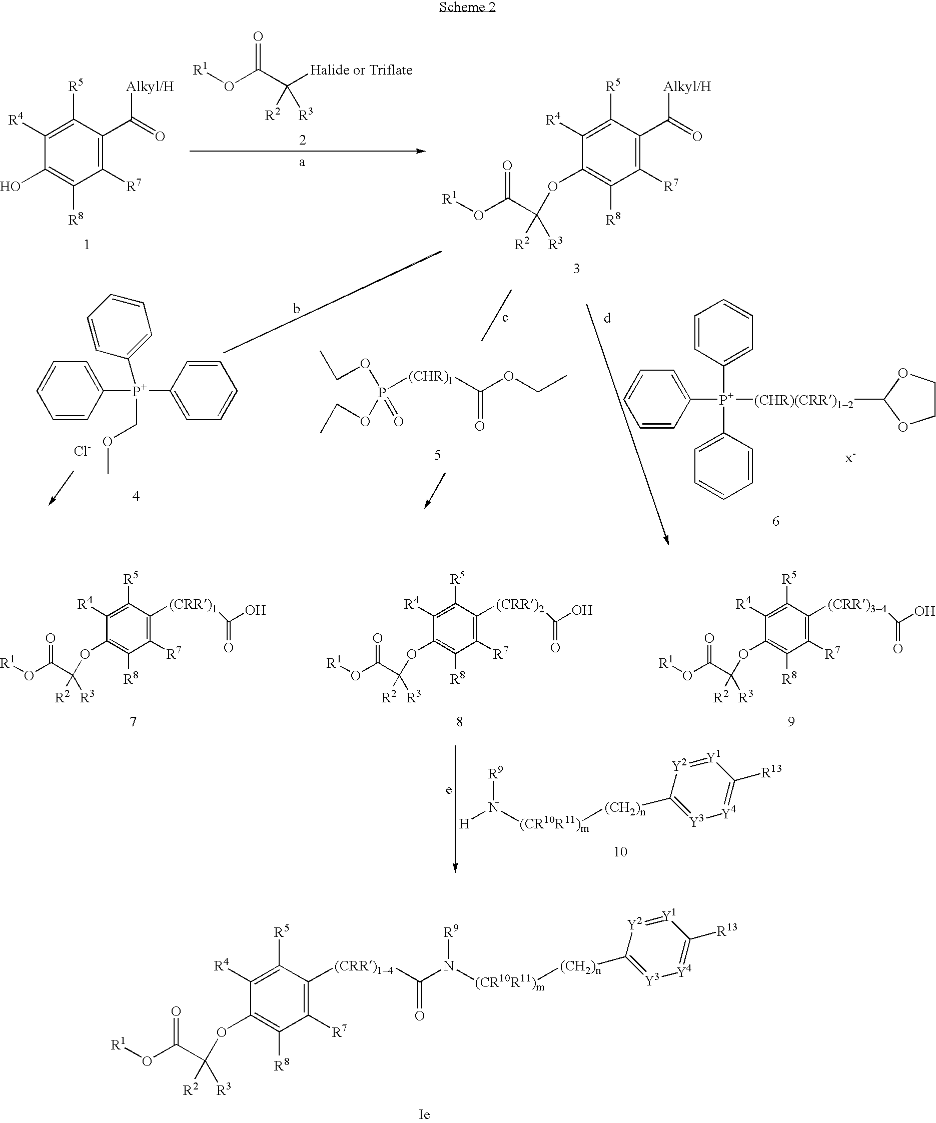

Aldehyde or ketone phenols 1 are known or can be prepared by methods known in the art. Compounds 1 can be transformed into aldehydes or ketones 3 by reaction with activated esters compounds 2 in the presence of a base like potassium or cesium carbonate in solvents like acetone, methyl-ethyl ketone, acetonitrile or N,N-dimethylformamide in a temperature range between room temperature and 140° C. In case a specific ketone precursor 1 is not available, addition of the suitable Grignard reagent to a protected aldehyde compound 1, e.g. carrying a SEM protective group (2-trimethylsilanyl-ethoxymethyl) at the phenolic OH-function, followed by oxidation of the thus formed Grignard adduct, e.g. using m-chloro-perbenzoic acid, TEMPO (2,2,6,6-tetramethyl-piperidine I-oxyl) and tetrabutyl ammonium bromide in dichlormethane preferably between 0° C. and room temperature, and a standard deprotection reaction yields then the desired ketone compound 1. Aldehydes or ketones 3 can be converted into acids 7, 8, or 9 by the following reaction sequences: i) e.g. by Wittig reaction with compounds 4 as reagents e.g. with potassium tert-butoxide as base in a solvent like tetrahydrofurane followed by mild acidic hydrolysis and oxidation (e.g. sodium chlorite, sodium dihydrogenphosphate-dihydrate in tert-butanol/water 2:1 in the presence of 2-methyl-2-butene at room temperature) (step b); ii) e.g. by Horner reaction with compounds 5 as reagents e.g. with sodium hydride as base in a solvent like tetrahydrofurane and subsequent hydrogenation and hydrolysis of the ester function (step c); iii) e.g. by Wittig reaction with acetals 6 as reagents e.g. with potassium tert-butoxide as base in a solvent like tetrahydrofurane and subsequent hydrogenation of the double bond, hydrolysis of the acetal function and oxidation to the acid e.g. as described above (step d). Acids 7, 8, or 9 can be chiral and can optionally be separated into optically pure antipodes by methods well known in the art, e.g. chromatography on a chiral HPLC column.

Condensation of acids 7, 8, or 9 with amines 10 can be performed using well known procedures for amide formation, such as use of N-(3-dimethylaminopropyl)-N′-ethyl-carbodiimide-hydrochloride and optionally 4-dimethylamino-pyridine in dichloromethane at temperatures between 0° C. and room temperature yielding compounds of formula Ie (step e). Those can optionally be hydrolyzed according to standard procedures, e.g. by treatment with an alkali hydroxide like LiOH or NaOH in a polar solvent mixture like tetrahydrofurane/ethanol/water to give carboxylic acids Ie. In case R¹ is equal to tert-butyl, treatment with e.g. trifluoroacetic acid, anisole in a solvent like dichloromethane between room temperature and the reflux temperature of the solvents yields carboxylic acids Ie.

An analogous reaction scheme with the same reaction sequences applies for the isomeric compound series leading to compounds of general formula I, particularly compounds according to formula If:

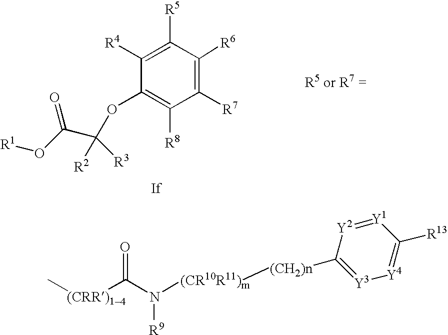

The synthesis of compounds with the general structure I, particularly compounds according to formula Ig with X¹ being an alkylene chain and X² equal to NR⁹CO can be accomplished according to scheme 3. Substituents R, R′ correspond to substituents as defined in detail in the claims.

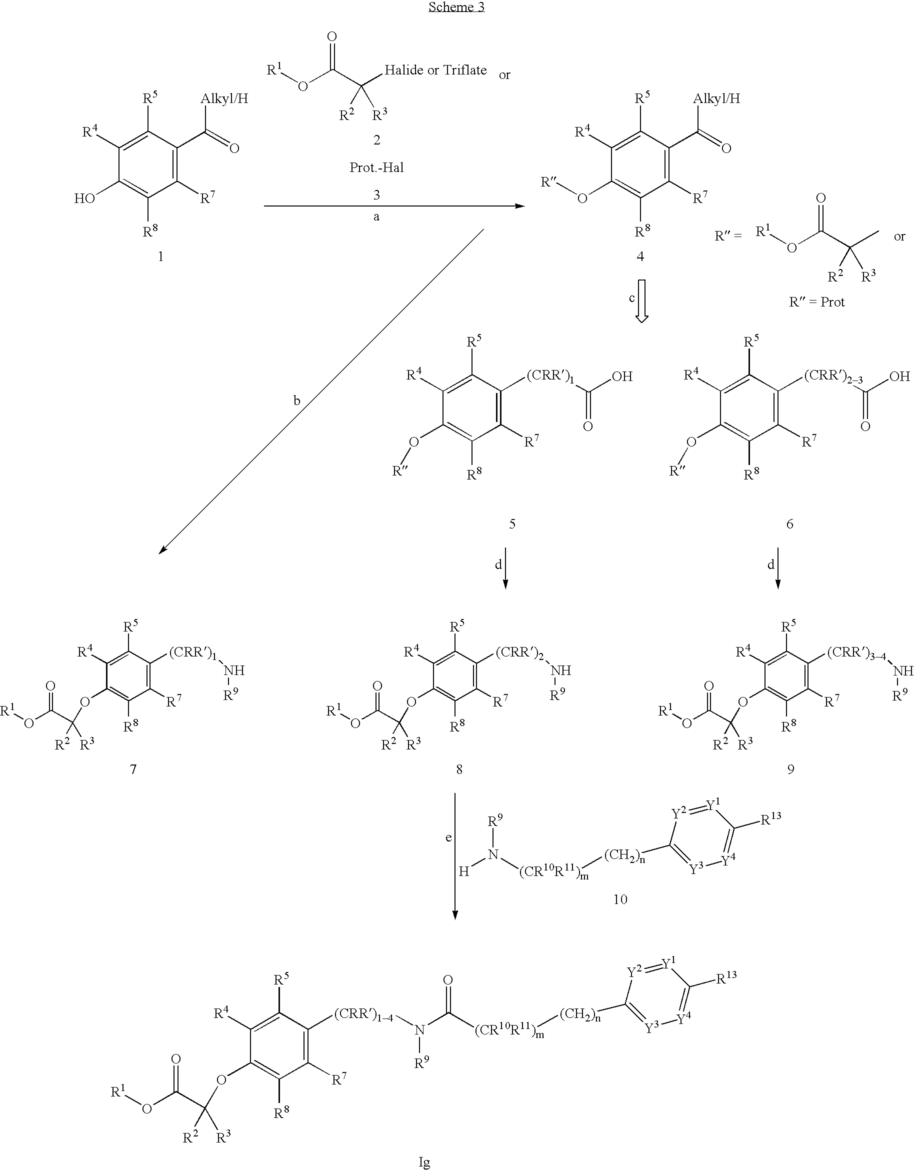

Aldehyde or ketone phenols 1 are known or can be prepared by methods known in the art. Compounds 1 can be transformed into aldehydes or ketones 4 by reaction with activated esters compounds 2 in the presence of a base like potassium or cesium carbonate in solvents like acetone, methyl-ethyl ketone, acetonitrile or N,N-dimethylformamide in a temperature range between room temperature and 140° C. Alternatively, a protective function can be attached to the phenolic hydroxy group of compounds 1, thus leading to compounds 4 with R″ equal to a protective group (step a). Depending on the synthetic route used, such a protective group can be removed at a later stage of the synthesis followed by attachment of activated esters compounds 2 as described above (see e.g. scheme 4). In case a specific ketone precursor 1 is not available, addition of the suitable Grignard reagent to a protected aldehyde compound 4, e.g. carrying a SEM (2-trimethylsilanyl-ethoxymethyl) protective group followed by oxidation of the thus formed Grignard adduct, e.g. using m-chloro-perbenzoic acid, TEMPO (2,2,6,6-tetramethylpiperidine 1-oxyl) and tetrabutyl ammonium bromide in dichlormethane preferably between 0° C. and room temperature, yields then the ketone compound 4 carrying a protective function at the phenolic moiety.

Aldehydes or ketones 4 can be converted into primary or secondary amine compounds 7 by oxime formation followed by reduction e.g. by catalytic hydrogenation in the presence of a platinum catalyst (step b). Ketones 4 can be converted into tertiary amine compounds 7 e.g. by imine formation with p-methoxy-benzylamine, addition of an organolithium or organo magnesium reagent followed by deprotection of the p-methoxy-benzylamine moiety with CAN (cerium (IV) ammonium nitrate). Conversion of amine compounds 7 into amine compounds 7 carrying a R⁹ substituent different from hydrogen can by performed by e.g. attachment of a BOC-protective function to the free amino group. BOC protected amine compounds 7 can be alkylated at nitrogen using sodium hydride and a reactive alkyl halogenide/mesylate or triflate to give, after standard BOC-deprotection (TFA/CH₂Cl₂, or HCl in dioxane at 0° C. to RT), compounds 7 carrying an R⁹ substituent different from hydrogen. Acids 5 or 6 can be prepared from suitably protected compounds 4 by reaction sequences as outlined for the preparation of acids 7, 8, and 9 in scheme 2 (step c). Acids 5 or 6 with R″ being a e.g. a 2-trimethylsilanyl-ethoxymethyl moiety can be converted into the aldehydes or alkyl ketones corresponding to compounds 4 with an optionally substituted alkylene chain between the aromatic moiety and the carbonyl function by standard Weinreb synthesis: i) Weinreb amide formation with methoxy-methylamine; ii) reaction with an organolithium reagent or diisobultylaluminium hydride. Such aldehyde and ketone precursors can be converted into amino compounds 8 or 9 with an optionally substituted alkylene chain between the NHR⁹ moiety and the central aromatic unit by a reaction sequence similar to that described above for the conversion compounds 4 into compounds 7 (step d). Amines 7, 8, or 9 can be chiral and can optionally be separated into optically pure antipodes by methods well known in the art, e.g. chromatography on a chiral HPLC column. Condensation of amines 7, 8, or 9 with acids 10 can be performed using well known procedures for amide formation, such as use of N-(3-dimethylaminopropyl)-N′-ethyl-carbodiimide-hydrochloride and optionally 4-dimethylamino-pyridine in dichloromethane at temperatures between 0° C. and room temperature yielding compounds Ig (step e). Those can optionally be hydrolyzed according to standard procedures, e.g. by treatment with an alkali hydroxide like LiOH or NaOH in a polar solvent mixture like tetrahydrofurane/ethanol/water to give carboxylic acids of formula Ig. In case R¹ is equal to tert-butyl, treatment with e.g. trifluoroacetic acid, anisole in a solvent like dichloromethane between room temperature and the reflux temperature of the solvents yields carboxylic acids Ig.

An analogous reaction scheme with the same reaction sequences applies for the isomeric compound series leading to compounds of general formula I, particularly compounds according to formula Ih:

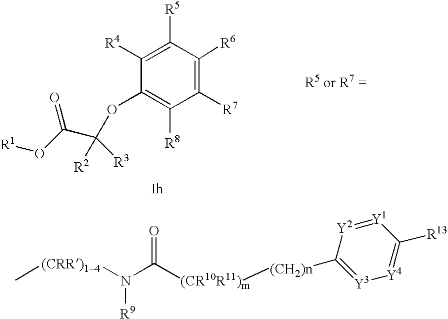

Scheme 4 describes the synthesis of intermediates with a tertiary carbon center in the alkylene chain between the central aromatic moiety and amide unit. These intermediates have not yet been described in schemes 1, 2 or 3.

Acids 1, corresponding to compounds 5, or 6 (scheme 3) or compound 9 (scheme 2, but carrying a protective function instead of the oxyacetic acid head group) can be mono- and or dialkylated at the carbon alpha to the acid function using standard enolate alkylation chemistry either with the acid via a dianion formed with e.g. a base like LDA or lithium hexamethyldisilazide in solvents like tetrahydrofurane or 1,2-dimethoxyethane, followed by addition of one or sequentially two different alkyl halides, a reaction preferably performed between −78° C. and room temperature followed by hydrolysis; or as an option, such a reaction can be performed with the corresponding ester via a mono-anions; thus, acids 2 are obtained directly or of after ester hydrolysis (step a). Chiral acids 2 can be prepared with high enantiomeric purity by using well known methodologies of enantioselective alkylation reactions as e.g. described in [Evans, David A.; et al. Journal of Organic Chemistry (1990), 55(26), 6260-8]: acids are converted into enantiomerically pure N-acyl 1,3-oxazolidine-2-ones followed by alkylation reaction with e.g. sodium hexamethyldisilazide as base and alkyl iodides as alkylating agents in solvents like tetrahydrofurane at temperatures around −78° C. and subsequent hydrolysis. In case, tertiary centers are formed, 0 alkylation might be predominant; thus, C-alkylated products can be formed from 0 alkylated products by reaction with methyl-aluminum-dichloride in a solvent like toluene at temperatures around −78° C. as described in [Suzuki, Tatsuo; et al. Tetrahedron Letters (2003), 44(18), 3713-3716].

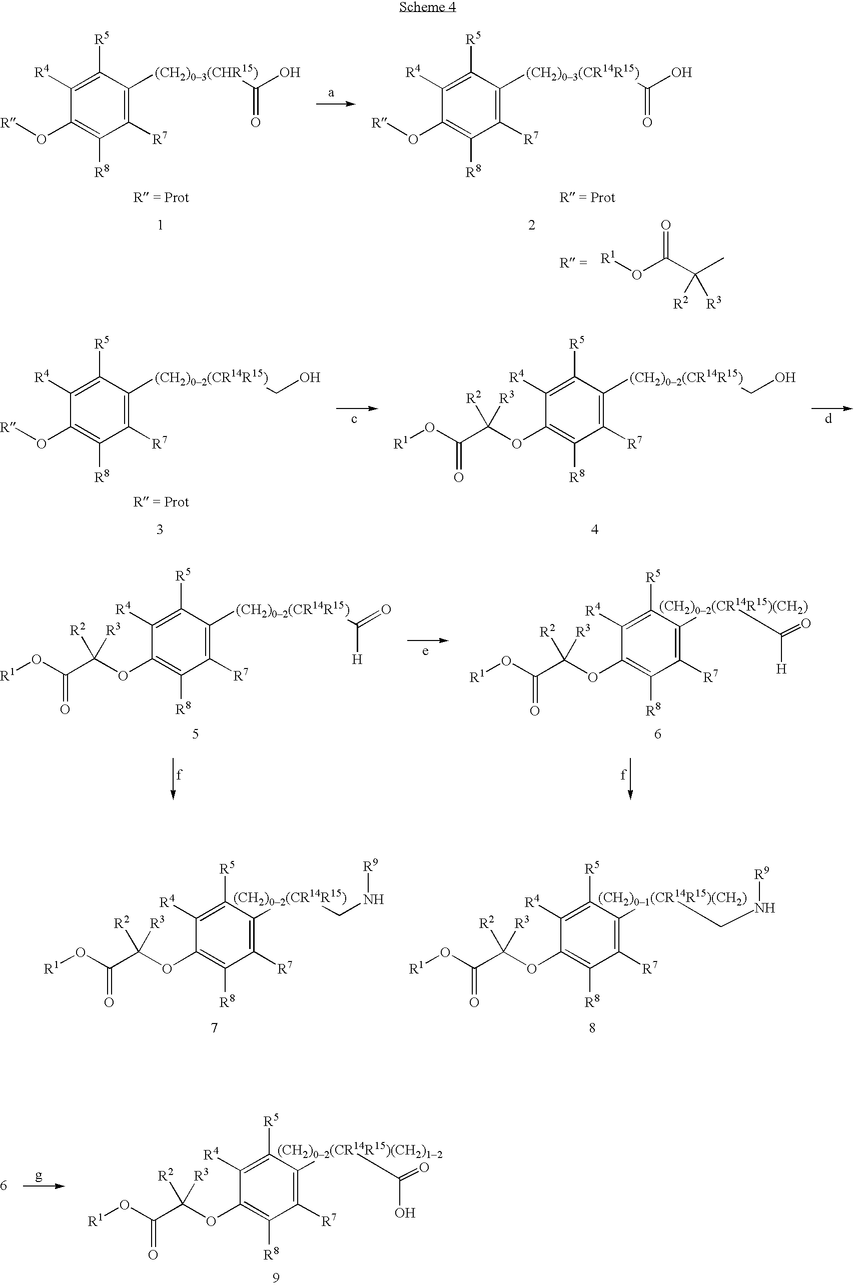

Acids 2 can be transformed into acids 2 with an alkoxyacetic acid head group and be used in amide forming reactions as described in schemes 1, 2 and 3 e.g. by: i) ester formation; ii) deprotection; iii) condensation with alpha halo tert-butyl esters as described in scheme 1; iv) selective ester hydrolysis. Alternatively, acids 2 can be reduced to the primary alcohol e.g. using borane/tetrahydrofurane as reagent (step b). Deprotection followed by condensation with alpha halo esters as described in scheme 1 gives then compounds 4 (step c).

Oxidation of compounds 4 e.g. using Swern conditions (oxalyl chloride/dimethylsulfoxide/triethylamine in dichloromethane, −78° C. to room temperature) gives compounds 5 (step d). Compounds 5 can optionally be elongated by one carbon by Wittig reaction using e.g. compound 4 (scheme 2) as reagent e.g. with potassium tert-butoxide as base in a solvent like tetrahydrofurane followed by mild acidic hydrolysis (step e). Optionally, this elongation procedure can be repeated with compounds 6 in order to introduce a second (CH₂) moiety. Aldehydes 5 and 6 can be converted into amino compounds 7 and 8 in analogy to the conversion described for compound 4 into compound 7 in scheme 3. Alternatively, compounds 6 or compounds 6 containing an additional (CH₂) group can be oxidized to the corresponding acids 9 e.g. using sodium chlorite, sodium dihydrogenphosphate-dihydrate in tert-butanol/water 2:1 in the presence of 2-methyl-2-butene at room temperature (step g). Amines 7 and 8, acids 2 with an alkoxyacetic acid head group and acids 9 can be chiral and can optionally be separated into optically pure antipodes by methods well known in the art, e.g. chromatography on a chiral HPLC column.

Amines 7 and 8 as well as acids 9 can be used in amide forming reactions as described in schemes 1, 2 and 3.

Scheme 5 to scheme 8 describe the synthesis of synthons 10 and 11 (scheme 1), of synthon 10 (scheme 2) and of synthon 10 (scheme 3).

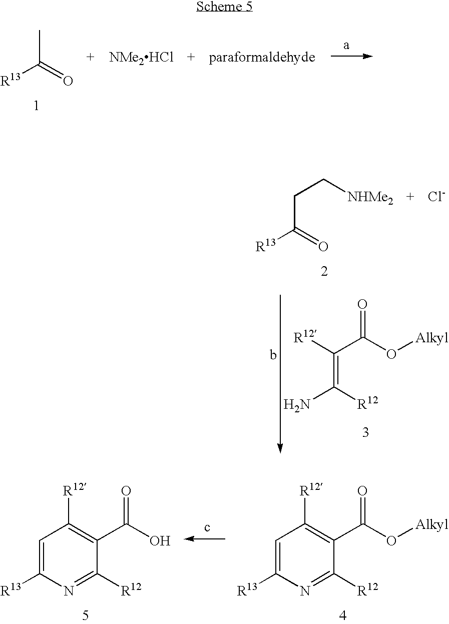

Pyridines 5 can be synthesized in a three step synthesis from ketones 1 (scheme 5). A mixture of ketones 1 with paraformaldehyde and dimethylamine hydrochloride in a solvent like ethanol in the presence of an acid like 37% HCl is heated to reflux for 2 to 10 hours to give aminoketones 2 (step a). Reaction of compounds 2 with 3-aminocrotonic acid esters 3 in acetic acid at reflux for 2 to 8 hours gives esters 4 (step b), which can be hydrolyzed (alkali hydroxide in solvents like THF, dioxane or DMSO) to give acids 5 (step c). Pyridines 4 can alternatively be synthesized following procedures described in [Al-Saleh, Balkis; Abdelkhalik, Mervat Mohammed; Eltoukhy, Afaf Mohammed; Elnagdi, Mohammed Hilmy. Enaminones in heterocyclic synthesis: A new regioselective synthesis of 2,3,6-trisubstituted pyridines, 6-substituted-3-aroylpyridines and 1,3,5-triaroylbenzenes. Journal of Heterocyclic Chemistry (2002), 39(5), 1035-1038]. Disubstituted pyridines 4 can be prepared according to procedures described in [Katsuyama, Isamu; Ogawa, Seiya; Yamaguchi, Yoshihiro; Funabiki, Kazumasa; Matsui, Masaki; Muramatsu, Hiroshige; Shibata, Katsuyoshi. A convenient and regioselective synthesis of 4-(trifluoromethyl)pyridines. Synthesis (1997), (11), 1321-1324].

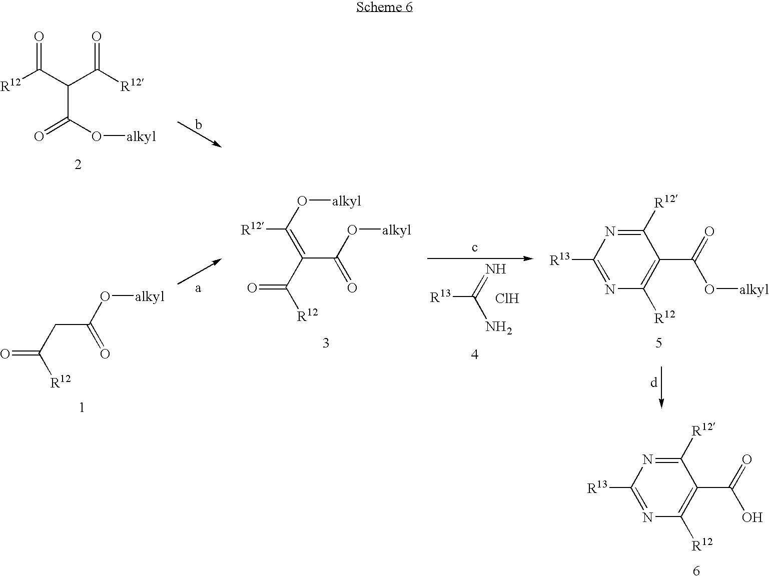

The synthesis of pyrimidine acids 6 is described in scheme 6. Reaction of 3-oxo-esters 1 with triethyl orthoformate in acetic anhydride at room temperature to reflux for 1 to 8 hours gives an E/Z mixture of the 3-ethoxy-acrylic acid esters 3 (step a). Diketo-esters 2 are reacted with methyl triflate in the presence of cesium carbonate in acetonitrile to give O-methylated products 3 (step b) [S. W. McCombie et al., Bioorganic & Medicinal Chemistry Letters 13 (2003), 567-571], thus yielding substituted enolethers 3 (R^{12′} not H). Reaction with amidine hydrochlorides 4 in ethanol in the presence of alkali tert-butoxide at room temperature gives access to esters 5 (step c). Esters 5 can be hydrolyzed (alkali hydroxide in solvents like THF, dioxane or DMSO) to give acids 6 (step d).

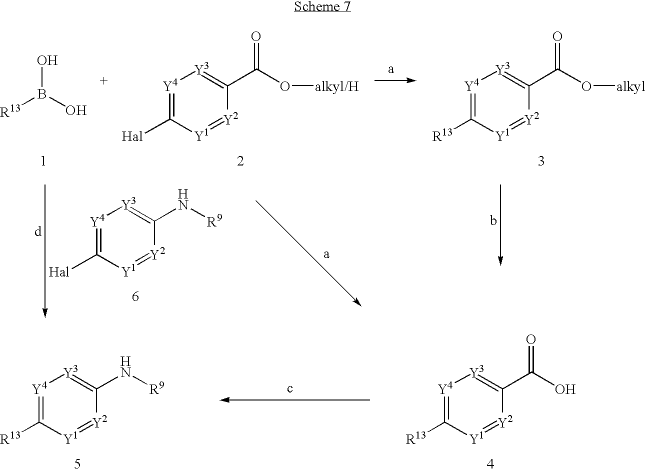

A general synthesis for acids 4 and amines 5 is depicted in scheme 7. Suzuki-coupling with boronic acids 1 and 4-halo-benzoic acid derivatives 2,6-halo-pyridazine-3-carboxylic acid derivatives 2,5-halo-pyrazine-2-carboxylic acid derivatives 2,6-halo-nicotinic acid derivatives 2, 5-halo-pyridine-2-carboxylic acid derivatives 2,2-halo-pyrimidine-5-carboxylic acid derivatives 2 or 5-halo-pyrimidine-2-carboxylic acid derivatives 2 or the corresponding optionally substituted halo-anilino compounds 6 with Pd(PhP)₄ or PdCl₂(dppf) [(1,1′-bis(diphenylphosphino)ferrocene)-dichloropalladium (II)×CH₂Cl₂ (1:1)] in toluene, dimethoxyethane, ethanol or DMF in the presence of cesium carbonate, potassium carbonate or cesium fluoride at room temperature to 90° C. for 2 to 8 h gives esters 3, acids 4 or anilines 5 (step a, d). Esters or acids 2 are either commercially available or can be prepared by methods known to a person skilled in the art. Esters 3 can be hydrolyzed (alkali hydroxide in solvents like THF, dioxane or DMSO) to give acids 4 (step b). A Curtius rearrangement can be used to transform acids 4 into the analogous BOC-protected anilines: first, the acid chlorides are synthesized with e.g. oxalyl chloride/DMF in dichloromethane. Then, reaction with sodium azide in DMF/dichloromethane followed by heating to reflux in the presence of 2-methyl-2-propanol gives the BOC protected anilines. Alternatively, such BOC protected anilines can be obtained from acids 4 in a one pot procedure by treatment with diphenylphosphoryl azide in 2-methyl-2-propanol in the presence of triethylamine and anhydrous 4-toluene sulfonic acid at temperatures around 100° C. Alkylation of these BOC protected anilines with an R⁹-halide in the presence of sodium hydride in solvents like DMF followed by BOC-deprotection with TFA or HCl in dioxane yields anilines 5 (step c).

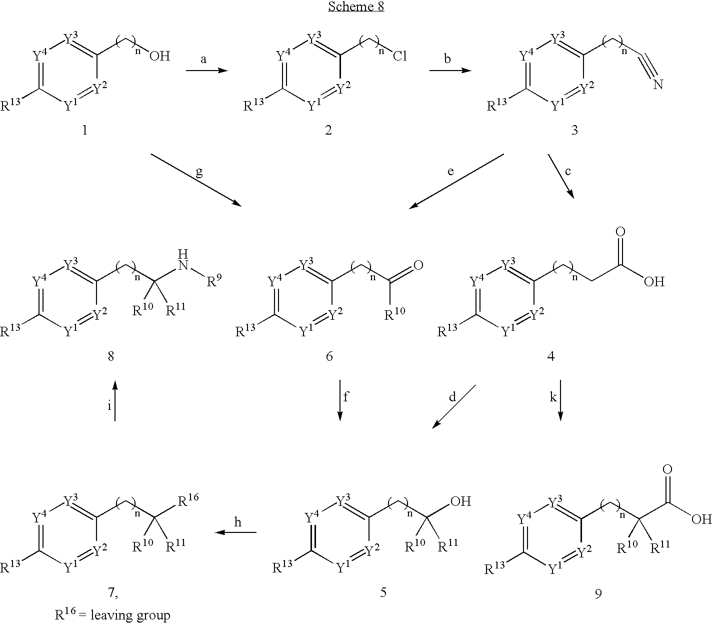

Alcohols 1 in scheme 8 comprising a chain length n equal to one [obtained by reduction of esters 3 (scheme 7) e.g. using diisobutylaluminium hydride-solution (in toluene) at −30° C. to room temperature for 30 min to 3 h in solvents like THF] can be converted into analogues with a chain length of n+1 carbon atoms by methods well known in the art, e.g. by conversion of the primary alcohol into a suitable leaving group, e.g. a halide (2, step a), followed by reaction with cyanide to form nitriles 3 (step b) and saponification to acids 4 (step c). Acids 4 can be further transformed into the primary alcohols 5 (R¹⁰=H, R¹¹=H), e.g. by using diborane in tetrahydrofurane (step d). Optionally, such alcohols 5 can be elongated to a chain length of n+1 carbon atoms by repeating the synthesis described for alcohols 1 to 5. In order to introduce substituents R¹⁰ and/or R¹¹ different from hydrogen, cyano intermediates 3 can be reacted with alkyl Grignard reagents R¹⁰MgX in solvents like ether or tetrahydrofurane between 0° C. and then reflux temperature of the solvent to form the corresponding R¹⁰CO-alkyl ketones 6 (step e) or with diisobutylaluminium hydride the corresponding aldehydes 6 (R¹⁰=H). Treatment of compounds 6 with an alkyllithium reagent R¹¹Li in solvents like ether or tetrahydrofurane gives alcohols 5 (step f); treatment of compounds 6 with lithium aluminium hydride in solvents like tetrahydrofurane or ether or with sodium borohydride in solvents like ethanol or methanol, preferably at temperatures between −15° C. and 40° C., gives alcohols 5 with R¹¹=H (step f). The alcohol compounds 5 which contain a chiral center can optionally be separated into optically pure antipodes by methods well known in the art, e.g. chromatography on a chiral HPLC column, or by derivatization with an optically pure acid to form esters, which can be separated by conventional HPLC chromatography and can then be converted back to the enantiomerically pure alcohols 5. The reduction of ketones 6 to the corresponding secondary alcohols 5 of scheme 8 can also be carried out in an enantioselective fashion leading to the (R)- or (S)-alcohols 5, e.g. by treatment with borane-dimethylsulfide complex and (S)- or (R)-2-methyl-CBS-oxazaborolidine as chiral catalyst in tetrahydrofurane, preferably at temperatures between −78° C. and ambient temperature, according to Corey et al. (E. J. Corey, R. K. Bakshi, S. Shibata, J. Am. Chem. Soc. 1987, 109, 5551-5553), or by treatment with (+)- or (−)-B-chlorodiisopinocampheyl-borane (DIP-Cl), according to Brown et al. (P. V. Ramachandran, B. Gong, A. V. Teodorovic, H. C. Brown, Tetrahedron: Asymmetry 1994, 5, 1061-1074). Aldehydes 6 (R¹⁰=H, n=0) can also be synthesized from primary alcohols 1 by methods known in the art, e.g. by treatment with pyridinium chlorochromate in dichloromethane, preferably at temperatures between room temperature and the reflux temperature of dichloromethane, or by treatment with manganese dioxide in solvents like dichloromethane, preferably at room temperature (step g). These aldehydes 6 can be converted to the corresponding secondary alcohols 5 through reaction with alkyl organometallic compounds, preferably under the conditions discussed above. Finally, the alcohols 5 of scheme 8 can be converted into compounds of formula 7, e.g by treatment with methanesulfonyl chloride in dichloromethane in the presence of a base like triethylamine preferably in a temperature range between −20° C. and room temperature or thionyl chloride in dichloromethane at 0° C. to room temperature or by reaction with carbon tetrachloride or carbon tetrabromide and triphenylphosphine in solvents like tetrahydrofurane preferably in a temperature range between room temperature and the reflux temperature of the solvents or by treatment with triflic anhydride, 2,6-lutidine and 4-dimethylaminopyridine in dichloromethane between −30° C. and room temperature; thus yielding compounds of formula 7 as methane-sulfonates, triflates, chlorides or bromides, respectively (step h). Compounds of formula 7 can further be converted (reaction step i) to the amines 8 in solvents like DMA, DMF or dichloromethane at room temperature with an excess of the corresponding amine.

Alpha mono- or di-substituted acids 9 (R¹⁰ and/or R¹¹ not H) can be synthesized via esters of compounds 4, by treatment with a base like LDA (lithium diisopropylamide) or lithium hexamethyldisilazide in solvents like tetrahydrofurane or 1,2-dimethoxyethane, followed by addition of one or sequentially two different alkyl halides, a reaction preferably performed between −78° C. and room temperature followed by hydrolysis to acid 9 (step k). Compounds 9 can be chiral and can optionally be separated into optically pure antipodes by methods well known in the art, e.g. chromatography on a chiral HPLC column, or by derivatization with an optically pure acid to form esters, which can be separated by conventional HPLC chromatography and then converted back to the enantiomerically pure alcohol. Additionally, the asymmetric alkylation can be done with chiral amides of 4 which are well known to a person skilled in the art.

Compounds of the general formula I can contain one or more stereocenters and can optionally be separated into optically pure enantiomers or diastereomers by methods well known in the art, e.g. by HPLC chromatography, chromatography on a chiral HPLC column, chromatography with a chiral eluant or by derivatization with an optically pure alcohol to form esters, which can be separated by conventional HPLC chromatography and then converted back to the enantiomerically pure acids I (R¹=H). In addition, racemic compounds can be separated into their antipodes via diastereomeric salts by crystallization with optically pure amines such as e.g. (R) or (S)-1-phenyl-ethylamine, (R) or (S)-1-naphthalen-1-yl-ethylamine, brucine, quinine or quinidine.

The following tests were carried out in order to determine the activity of the compounds of formula (I).

Background information on the performed assays can be found in: Nichols J S et al. “Development of a scintillation proximity assay for peroxisome proliferator-activated receptor gamma ligand binding domain”, (1998) Anal. Biochem. 257: 112-119.

Full-length cDNA clones for humans PPARδ and PPARα and mouse PPARγ were obtained by RT-PCR from human adipose and mouse liver cRNA, respectively, cloned into plasmid vectors and verified by DNA sequencing. Bacterial and mammalian expression vectors were constructed to produce glutathione-s-transferase (GST) and Gal4 DNA binding domain proteins fused to the ligand binding domains (LBD) of PPARδ (aa 139 to 442), PPARγ (aa 174 to 476) and PPARα (aa 167 to 469). To accomplish this, the portions of the cloned sequences encoding the LBDs were amplified from the full-length clones by PCR and then subcloned into the plasmid vectors. Final clones were verified by DNA sequence analysis.

Induction, expression, and purification of GST-LBD fusion proteins were performed in E. coli strain BL21 (pLysS) cells by standard methods (Ref: Current Protocols in Molecular Biology, Wiley Press, edited by Ausubel et al.).

Radioligand Binding Assay

PPARδ receptor binding was assayed in HNM10 (50 mM Hepes, pH 7.4, 10 mM NaCl, 5 mM MgCl₂, 0.15 mg/ml fatty acid-free BSA and 15 mM DTT). For each 96 well reaction a 500 ng equivalent of GST-PPARδ-LBD fusion protein and radioligand, e.g. 20000 dpm {2-methyl-4-[4-methyl-2-(4-trifluoromethyl-phenyl)-thiazol-5-yl-ditritiomethylsulfanyl]-phenoxy}-acetic acid, was bound to 10 μg SPA beads (PharmaciaAmersham) in a final volume of 50 μl by shaking. The resulting slurry was incubated for 1 h at RT and centrifuged for 2 min at 1300 g. The supernatant containing unbound protein was removed and the semidry pellet containing the receptor-coated beads was resuspended in 50 μl of HNM. Radioligand was added and the reaction incubated at RT for 1 h and scintillation proximity counting performed in the presence of test compounds was determined. All binding assays were performed in 96 well plates and the amount of bound ligand was measured on a Packard TopCount using OptiPlates (Packard). Dose response curves were done in triplicates within a range of concentration from 10⁻¹⁰ M to 10⁻⁴M.

PPARα receptor binding was assayed in TKE50 (50 mM Tris-HCl, pH 8, 50 mM KCl, 2 mM EDTA, 0.1 mg/ml fatty acid-free BSA and 10 mM DTT). For each 96 well reaction an 140 ng equivalent of GST-PPARα-LBD fusion protein was bound to 10 μg SPA beads (PharmaciaAmersham) in a final volume of 50 μl by shaking. The resulting slurry was incubated for 1 h at RT and centrifuged for 2 min at 1300 g. The supernatant containing unbound protein was removed and the semidry pellet containing the receptor-coated beads was resolved in 50 μl of TKE. For radioligand binding e.g. 10000 dpm of 2(S)-(2-benzoyl-phenylamino)-3-{4-[1,1-ditritio-2-(5-methyl-2-phenyl-oxazol-4-yl)-ethoxy]-phenyl}-propionic acid or 2,3-ditritio-2(S)-methoxy-3-{4-[2-(5-methyl-2-phenyl-oxazol-4-yl)-ethoxy]-benzo[b]thiophen-7-yl}-propionic acid in 50 ul were added, the reaction incubated at RT for 1 h and scintillation proximity counting performed. All binding assays were performed in 96 well plates and the amount of bound ligand measured on a Packard TopCount using OptiPlates (Packard). Nonspecific binding was determined in the presence of 10⁻⁴ M unlabelled compound. Dose response curves were done in triplicates within a range of concentration from 10⁻¹⁰ M to 10⁻⁴ M.

PPARγ receptor binding was assayed in TKE50 (50 mM Tris-HCl, pH 8, 50 mM KCl, 2 mM EDTA, 0.1 mg/ml fatty acid-free BSA and 10 mM DTT). For each 96 well reaction an 140 ng equivalent of GST-PPARγ-LBD fusion protein was bound to 10 μg SPA beads (PharmaciaAmersham) in a final volume of 50 ul by shaking. The resulting slurry was incubated for 1 h at RT and centrifuged for 2 min at 1300 g. The supernatant containing unbound protein was removed and the semidry pellet containing the receptor-coated beads was resolved in 50 ul of TKE. For radioligand binding e.g. 10000 dpm 2(S)-(2-benzoyl-phenylamino)-3-{4-[1,1-ditritio-2-(5-methyl-2-phenyl-oxazol-4-yl)-ethoxy]-phenyl}-propionic acid in 50 μl were added, the reaction incubated at RT for 1 h and scintillation proximity counting performed. All binding assays were performed in 96 well plates and the amount of bound ligand measured on a Packard TopCount using OptiPlates (Packard). Nonspecific binding was determined in the presence of 10⁻⁴ M unlabelled compound. Dose response curves were done in triplicates within a range of concentration from 10⁻¹⁰ M to 10⁻⁴ M.

Luciferase Transcriptional Reporter Gene Assays

Baby hamster kidney cells (BHK21 ATCC CCL10) were grown in DMEM medium containing 10% FBS at 37° C. in a 95% O2:5% CO₂ atmosphere. Cells were seeded in 6 well plates at a density of 10⁵ Cells/well and then batch-transfected with either the pFA-PPARδ-LBD, pFA-PPARγ-LBD or pFA-PPARα-LBD expression plasmids plus a reporter plasmid. Transfection was accomplished with the Fugene 6 reagent (Roche Molecular Biochemicals) according to the suggested protocol. Six hours following transfection, the cells were harvested by trypsinization and seeded in 96 well plates at a density of 10⁴ cells/well. After 24 hours to allow attachment of cells, the medium was removed and replaced with 100 ul of phenol red-free medium containing the test substances or control ligands (final DMSO concentration: 0.1%). Following incubation of the cells for 24 hours with substances, 50 ul of the supernatant was discarded and then 50 μl of Luciferase Constant-Light Reagent (Roche Molecular Biochemicals) to lyse the cells and initiate the luciferase reaction was added. Luminescence for luciferase was measured in a Packard TopCount. Transcriptional activation in the presence of a test substance was expressed as fold-activation over cells incubated in the absence of the substance. EC50 values were calculated using the XLfit program (ID Business Solutions Ltd. UK).

The free acids of the compounds of the present invention (R¹ is hydrogen) exhibit IC₅₀ values of 0.5 nM to 10 μM, preferably 1 nM to 100 nM for PPARα and/or IC₅₀ values of 1 nM to 10 μM, preferably 10 nM to 5 μM for PPARδ and/or IC₅₀ values of 100 nM to 10 μM, preferably 500 nM to 5 μM for PPARγ. Compounds, in which R¹ is not hydrogen are converted in vivo to compounds in which R¹ is hydrogen. The following table shows measured values for selected compounds of the present invention.

| | PPARα | PPARγ | PPARδ |
|---|---|---|---|
| | IC₅₀ (μmol/l) | IC₅₀ (μmol/l) | IC₅₀ (μmol/l) |
| Example 09 | 0.42 | >10 | 0.58 |
| Example 16 | 0.008 | >10 | >10 |
| Example 20 | 0.024 | 1.08 | 0.94 |

The compounds of formula (I) and their pharmaceutically acceptable salts and esters can be used as medicaments, e.g. in the form of pharmaceutical preparations for enteral, parenteral or topical administration. They can be administered, for example, perorally, e.g. in the form of tablets, coated tablets, dragées, hard and soft gelatine capsules, solutions, emulsions or suspensions, rectally, e.g. in the form of suppositories, parenterally, e.g. in the form of injection solutions or infusion solutions, or topically, e.g. in the form of ointments, creams or oils.

The production of the pharmaceutical preparations can be effected in a manner which will be familiar to any person skilled in the art by bringing the described compounds of formula (I) and their pharmaceutically acceptable, into a galenical administration form together with suitable, non-toxic, inert, therapeutically compatible solid or liquid carrier materials and, if desired, usual pharmaceutical adjuvants.

Suitable carrier materials are not only inorganic carrier materials, but also organic carrier materials. Thus, for example, lactose, corn starch or derivatives thereof, talc, stearic acid or its salts can be used as carrier materials for tablets, coated tablets, dragées and hard gelatine capsules. Suitable carrier materials for soft gelatine capsules are, for example, vegetable oils, waxes, fats and semi-solid and liquid polyols (depending on the nature of the active ingredient no carriers are, however, required in the case of soft gelatine capsules). Suitable carrier materials for the production of solutions and syrups are, for example, water, polyols, sucrose, invert sugar and the like. Suitable carrier materials for injection solutions are, for example, water, alcohols, polyols, glycerol and vegetable oils. Suitable carrier materials for suppositories are, for example, natural or hardened oils, waxes, fats and semi-liquid or liquid polyols. Suitable carrier materials for topical preparations are glycerides, semi-synthetic and synthetic glycerides, hydrogenated oils, liquid waxes, liquid paraffins, liquid fatty alcohols, sterols, polyethylene glycols and cellulose derivatives.

Usual stabilizers, preservatives, wetting and emulsifying agents, consistency-improving agents, flavour-improving agents, salts for varying the osmotic pressure, buffer substances, solubilizers, colorants and masking agents and antioxidants come into consideration as pharmaceutical adjuvants.

The dosage of the compounds of formula (I) can vary within wide limits depending on the disease to be controlled, the age and the individual condition of the patient and the mode of administration, and will, of course, be fitted to the individual requirements in each particular case. For adult patients a daily dosage of about 0.1 mg to about 1000 mg, especially about 1 mg to about 100 mg, comes into consideration. Depending on the dosage it is convenient to administer the daily dosage in several dosage units.

The pharmaceutical preparations conveniently contain about 0.1-500 mg, preferably 0.5-100 mg, of a compound of formula (I).

The following examples serve to illustrate the present invention in more detail. They are, however, not intended to limit its scope in any manner.

### EXAMPLES

Abbreviations:

AcOEt=ethyl acetate, n-BuLi=n-butyllithium, DBU=1,8-diazabicyclo[5.4.0]undec-7-ene, MeCl₂=dichloromethane; DEAD=diethyl azodicarboxylate, DIAD=diisopropyl azodicarboxylate, DIBAL-H solution=diisobutylaluminum hydride solution, DMF=N,N-dimethylformamide, DMPU=1,3-dimethyl-3,4,5,6-tetrahydro-2(1H)-pyrimidinone, eq.=equivalents, h=hour(s), DMSO=dimethyl sulfoxide, HPLC=high performance liquid chromatography, i. V.=in vacuo, LDA=lithium diisopropylamide, PdCl₂(dppf)=(1,1′-bis(diphenylphosphino)ferrocene)dichloro-palladium(II).CH₂Cl₂ (1:1), Pd(Ph₃P)₄=tetrakis(triphenylphosphine)palladium, POCl₃=phosphorus oxychloride, RT=room temperature, TFA=trifluoroacetic acid, TFAA=trifluoroacetic anhydride, THF=tetrahydrofurane.

### Example 1

### [rac]-2-[4-(1-{[4-Cyclopropyl-2-(4-trifluoromethyl-phenyl)-pyrimidine-5-carbonyl]-amino}-ethyl)-2-methyl-phenoxy]-2-methyl-propionic acid

### A] [rac]-2-[4-(1-{[4-Cyclopropyl-2-(4-trifluoromethyl-phenyl)-pyrimidine-5-carbonyl]-amino}-ethyl)-2-methyl-phenoxy]-2-methyl-propionic acid ethyl ester

0.25 g (0.94 mmol) of [rac]-2-[4-(1-amino-ethyl)-2-methyl-phenoxy]-2-methyl-propionic acid ethyl ester [PCT Int. Appl. (2002), 35 pp. WO 2002096894A1] and 0.29 g (1.00 mmol) of 4-cyclopropyl-2-(4-trifluoromethyl-phenyl)-pyrimidine-5-carboxylic acid (example 1E]) were dissolved in 10 ml of CH₂Cl₂. To this solution were added 0.22 g (1.13 mmol) of N-(3-dimethylamino-propyl)-N′-ethyl-carbodiimide-hydrochloride and 0.15 g (1.22 mmol) of N,N-dimethylaminopyridine and this mixture was stirred for 20 hours at RT. The solvent was removed by evaporation and the crude product was purified by chromatography (SiO₂; n-heptane/AcOEt=4:1 to 1:1) to give 0.42 g of the title compound as a colorless foam.

MS: 556.2 (M+H)⁺.

### B] [rac]-2-[4-(1-{[4-Cyclopropyl-2-(4-trifluoromethyl-phenyl)-pyrimidine-5-carbonyl]-amino}-ethyl)-2-methyl-phenoxy]-2-methyl-propionic acid

0.40 g (0.72 mmol) of the above prepared [rac]-2-[4-(1-{[4-cyclopropyl-2-(4-trifluoromethyl-phenyl)-pyrimidine-5-carbonyl]-amino}-ethyl)-2-methyl-phenoxy]-2-methyl-propionic acid ethyl ester was dissolved in 7.5 ml of THF/MeOH=2:1. To the stirred solution was added 2.16 ml (2.16 mmol) of a LiOH-solution (1M in water). After 16 hours, the reaction mixture was poured into crashed ice/HCl and extracted twice with CH₂Cl₂; the organic layers were washed with water, dried over magnesium sulfate, filtered and evaporated to give 0.40 g of crude product. Recrystallization from AcOEt/n-heptane gave 0.30 g of pure title compound as colorless solid.

MS: 528.4 (M+H)⁺.

The 4-cyclopropyl-2-(4-trifluoromethyl-phenyl)-pyrimidine-5-carboxylic acid used in 1A] was synthesized as follows:

### C] (E,Z)-2-Cyclopropanecarbonyl-3-ethoxy-acrylic acid methyl ester

A solution of 10 g (70.34 mmol) 3-cyclopropyl-3-oxo-propionic acid methyl ester and of 23.4 ml (140.68 mmol) of triethyl orthoformate in 100 ml acetic anhydride was refluxed at 150° C. for 5 h. The reaction mixture was concentrated at 95° C. under reduced pressure to give 14.35 g of crude (E,Z)-2-cyclopropanecarbonyl-3-ethoxy-acrylic acid methyl ester.

MS: 199.3 (M+H)⁺.

### D] 4-Cyclopropyl-2-(4-trifluoromethyl-phenyl)-pyrimidine-5-carboxylic acid ethyl ester

To a solution of 4.74 g (18.19 mmol) 4-trifluoromethyl-benzamidine HCl in 50 ml of ethanol was added 1.818 g (18.186 mmol) of sodium tert-butoxide. After 2 min, 3.605 g of crude (E,Z)-2-cyclopropanecarbonyl-3-ethoxy-acrylic acid methyl ester was added and the reaction mixture was then stirred over night at RT. The ethanol was removed under reduced pressure, the residue taken up in ether and washed with 1N HCl and water. The ether solution was concentrated under reduced pressure and the crude product purified by chromatography over silica gel with AcOEt/heptane 1:3 to give 4.25 g of pure 4-cyclopropyl-2-(4-trifluoromethyl-phenyl)-pyrimidine-5-carboxylic acid ethyl ester.

MS: 337.1 (M+H)⁺.

### E] 4-Cyclopropyl-2-(4-trifluoromethyl-phenyl)-pyrimidine-5-carboxylic acid

The solutions of 3.6 g (10.7 mmol) of 4-cyclopropyl-2-(4-trifluoromethyl-phenyl)-pyrimidine-5-carboxylic acid ethyl ester in 40 ml of ethanol and of 1.07 g (26.7 mmol) of sodium hydroxide in 5 ml of H₂O were mixed and then refluxed for 1 hour. After cooling to ambient temperature, 6.7 ml of 4N aqueous hydrochloric acid was added. The reaction mixture was extracted with three portions of ethyl acetate. The combined organic phases were washed with water and brine and dried over anhydrous sodium sulfate. The 4-cyclopropyl-2-(4-trifluoromethyl-phenyl)-pyrimidine-5-carboxylic acid crystallized upon concentrating the solution by evaporation. After cooling in an ice bath, 3.08 g of white crystals were obtained.

MS: 307.2 (M−H)⁻.

### Example 2

### [rac]-2-[4-(1-{[4-Cyclopropyl-2-(3-trifluoromethyl-phenyl)-pyrimidine-5-carbonyl]-amino}-ethyl)-2-methyl-phenoxy]-2-methyl-propionic acid

A] In analogy to the procedures described in example 1A] and 1B], [rac]-2-[4-(1-amino-ethyl)-2-methyl-phenoxy]-2-methyl-propionic acid ethyl ester [PCT Int. Appl. (2002), 35 pp. WO 2002096894A1] was reacted with 4-cyclopropyl-2-(3-trifluoromethyl-phenyl)-pyrimidine-5-carboxylic acid (example 2C]) to give [rac]-2-[4-(1-{[4-cyclopropyl-2-(3-trifluoromethyl-phenyl)-pyrimidine-5-carbonyl]-amino}-ethyl)-2-methyl-phenoxy]-2-methyl-propionic acid ethyl ester, which was subsequently saponified to yield the title compound as colorless solid.

MS: 528.4 (M+H)⁺.

The 4-cyclopropyl-2-(3-trifluoromethyl-phenyl)-pyrimidine-5-carboxylic acid used in 2A] was synthesized as follows:

### B] 4-Cyclopropyl-2-(3-trifluoromethyl-phenyl)-pyrimidine-5-carboxylic acid ethyl ester

To a solution of 0.953 g (4.24 mmol) commercially available 3-trifluoromethyl-benzamidine hydrochloride in 10 ml of ethanol was added 0.408 g (4.25 mmol) of sodium tert-butoxide. Two min. later, 0.901 g (4.25 mmol) of crude (E,Z)-2-cyclopropane-carbonyl-3-ethoxy-acrylic acid methyl ester (example 1C], containing some Et-ester) was added and the reaction allowed to proceed over night at RT. The mixture was then poured onto crashed ice/AcOEt/HCl dil., the aqueous phase extracted again with AcOEt, the combined organic layers were washed with water, dried over sodium sulfate, and evaporated to dryness. Flash chromatography (SiO₂, hexane/AcOEt=9/1) yielded finally 1.253 g of title compound as white waxy solid (mixture of Me/Et− ester).

MS: 322.1, 336.0 (M)⁺.

### C] 4-Cyclopropyl-2-(3-trifluoromethyl-phenyl)-pyrimidine-5-carboxylic acid

In analogy to the procedure described in examples 1E], 4-cyclopropyl-2-(3-trifluoromethyl-phenyl)-pyrimidine-5-carboxylic acid ethyl ester was saponified to yield the title compound as colorless solid.

MS: 307.2 (M−H)⁻.

### Example 3

### [rac]-2-Methyl-2-(2-methyl-4-{1-[(3′-trifluoromethyl-biphenyl-4-carbonyl)-amino]-ethyl}-phenoxy)-propionic acid

A] In analogy to the procedures described in example 1A] and 1B], [rac]-2-[4-(1-amino-ethyl)-2-methyl-phenoxy]-2-methyl-propionic acid ethyl ester [PCT Int. Appl. (2002), 35 pp. WO 2002096894A1] was reacted with 3′-trifluoromethyl-biphenyl-4-carboxylic acid (example 3B]) to give [rac]-2-methyl-2-(2-methyl-4-{1-[(3′-trifluoromethyl-biphenyl-4-carbonyl)-amino]-ethyl}-phenoxy)-propionic acid ethyl ester, which was subsequently saponified to yield the title compound as colorless solid.

MS: 484.4 (M−H)⁻.

The 3′-trifluoromethyl-biphenyl-4-carboxylic acid used in 3A] was synthesized as follows:

### B] 3′-Trifluoromethyl-biphenyl-4-carboxylic acid

3.0 g (12.1 mmol) of 4-iodo benzoic acid was dissolved in 40 ml of 1,2-dimethoxy-ethane, 20 ml of water was added, followed by 2.44 g (12.5 mmol) of 3-(trifluoromethyl)-benzeneboronic acid, 2.27 g (20.8 mmol) of sodium carbonate and 0.28 g (0.24 mmol) of tetrakis(triphenylphosphine)palladium. This mixture was stirred for 2 hours at 95° C., cooled down to RT and filtered. The pH of this solution was adjusted with HCl (1N) to pH 1-2, and it was then extracted twice with AcOEt. The organic layers were washed with water, dried over magnesium sulfate, filtered and evaporated to give 3.58 g crude product, which was purified by chromatography over silica gel with a gradient of MeCl₂ and MeOH to give 2.70 g of the title compound as light yellow solid.

MS: 265.0 (M-H)—.

### Example 4

### [rac]-2-Methyl-2-(2-methyl-4-{1-[(4′-trifluoromethyl-biphenyl-4-carbonyl)-amino]-ethyl}-phenoxy)-propionic acid

In analogy to the procedures described in example 1A] and 1B], [rac]-2-[4-(1-amino-ethyl)-2-methyl-phenoxy]-2-methyl-propionic acid ethyl ester [PCT Int. Appl. (2002), 35 pp. WO 2002096894A1] was reacted with 4′-trifluoromethyl-biphenyl-4-carboxylic acid (prepared in analogy to the procedure described in example 3B]) to give [rac]-2-methyl-2-(2-methyl-4-{1-[(4′-trifluoromethyl-biphenyl-4-carbonyl)-amino]-ethyl}-phenoxy)-propionic acid ethyl ester, which was subsequently saponified to yield the title compound as colorless solid.

MS: 484.3 (M−H)⁻.

### Example 5

### [rac]-2-Methyl-2-(2-methyl-4-{1-[2-methyl-6-(4-trifluoromethyl-phenyl)-pyridin-3-ylcarbamoyl]-ethoxy}-phenoxy)-propionic acid

A] In analogy to the procedures described in example 1A] and 1B], [rac]-2-[4-(1-carboxy-ethoxy)-2-methyl-phenoxy]-2-methyl-propionic acid ethyl ester (example 5G]) was reacted with 2-methyl-6-(4-trifluoromethyl-phenyl)-pyridin-3-ylamine (example 5E]) to give [rac]-2-methyl-2-(2-methyl-4-{1-[2-methyl-6-(4-trifluoromethyl-phenyl)-pyridin-3-ylcarbamoyl]-ethoxy}-phenoxy)-propionic acid ethyl ester, which was subsequently saponified to yield the title compound as colorless solid.

MS: 515.2 (M−H)⁻.

The necessary building block 2-methyl-6-(4-trifluoromethyl-phenyl)-pyridin-3-ylamine used in the procedure above was prepared as follows:

### B] 3-Dimethylamino-1-(4-trifluoromethyl-phenyl)-propan-1-one hydrochloride

4-(Trifluoromethyl) acetophenone (4.97 g, 26.4 mmol), paraformaldehyde (1.586 g, 2 eq.) and dimethylamine hydrochloride (3.231 g, 1.5 eq.) were mixed together in 7 ml of EtOH, treated with 0.08 ml of 37% HCl, and heated to reflux for 5 h. Cooling down to ambient temperature, filtration and washing with tiny amounts of cold EtOH delivered 4.59 g of the title compound as white crystals, mp. 128-42° C. (dec.).

MS: 246.3 (M+H)⁺.

### C] 2-Methyl-6-(4-trifluoromethyl-phenyl)-nicotinic acid methyl ester

4.59 g (16.3 mmol) of the above prepared 3-dimethylamino-1-(4-trifluoromethyl-phenyl)-propan-1-one hydrochloride and 1.86 g (1.0 eq.) of 3-aminocrotonic acid methyl ester were dissolved in 50 ml of AcOH and heated to reflux for 4 h. After cooling, the bulk of the solvent was evaporated i. V., the residue dissolved in AcOEt, and washed with water and brine. Drying over sodium sulfate, evaporation of the solvents and flash chromatography (SiO₂, hexane/AcOEt=8/2) delivered finally 2.40 g of the title compound as light yellow waxy solid.

MS: 296.1 (M+H)⁺.

### D] [2-Methyl-6-(4-trifluoromethyl-phenyl)-pyridin-3-yl]-carbamic acid tert-butyl ester

4.30 g (15.3 mmol) of 2-methyl-6-(4-trifluoromethyl-phenyl)-nicotinic acid (prepared from 2-methyl-6-(4-trifluoromethyl-phenyl)-nicotinic acid methyl ester in analogy to the procedure described in example 1E]) was dissolved in 85 ml of 2-methyl-2-propanol and 3.18 ml=2.32 g (22.9 mmol) of triethylamine was added. After 5 min., 4.97 ml=6.64 g (22.9 mmol) of diphenylphosphoryl azide (95%) was added. The reaction mixture was then stirred at reflux (oil bath 100° C.). After 10 min., 0.53 g (3.1 mmol) of anhydrous 4-toluene sulfonic acid was added and stirring continued for 1 hour at reflux. The solvent was then completely removed by evaporation at high vacuum; the residue was dissolved in Et₂O and washed with H₂O, 1N HCl, and NaHCO₃ solution. The combined organic phases were dried over anhydrous magnesium sulfate and evaporated. The crude product was purified by crystallization (EtOAc, n-heptane) to give 4.05 g of the title compound as colorless solid.

MS: 353.3 (M+H)⁺.

### E] 2-Methyl-6-(4-trifluoromethyl-phenyl)-pyridin-3-ylamine

To a solution of 2.0 g (5.68 mmol) of [2-methyl-6-(4-trifluoromethyl-phenyl)-pyridin-3-yl]-carbamic acid tert-butyl ester in 25 ml of MeCl₂ were added (drop by drop) 2.17 ml (28.4 mmol) of trifluoroacetic acid at RT. After 20 hours, the solvent was removed by evaporation in vacuo, the residue was poured into crashed ice, the pH was adjusted to >12 with NaOH (1N) and the mixture was extracted three times with Et₂O; the organic phases were washed with water, dried with MgSO₄, filtered and evaporated to give 1.60 g of crude product. Purification by flash chromatography on SiO₂ with a gradient of n-heptane: AcOEt (9:1 to 1:1) yielded 1.27 g of 2-methyl-6-(4-trifluoromethyl-phenyl)-pyridin-3-ylamine as colorless solid

MS: 253.1 (M+H)⁺.

The necessary building block [rac]-2-[4-(1-carboxy-ethoxy)-2-methyl-phenoxy]-2-methyl-propionic acid ethyl ester used in the procedure above was prepared as follows:

### F] [rac]-2-[4-(1-Methoxycarbonyl-ethoxy)-2-methyl-phenoxy]-2-methyl-propionic acid ethyl ester

A mixture of 3.0 g (12.6 mmol) of 2-(4-hydroxy-2-methyl-phenoxy)-2-methyl-propionic acid ethyl ester (described in WO 02/092590), 3.96 ml=4.51 g (33.2 mmol) of methyl-2-chloro-propionate, 0.42 g (2.5 mmol) of potassium iodide and 8.70 g (63 mmol) of K₂CO₃ in 60 ml of DMF was stirred for 54 h at RT. It was then poured into crashed ice and extracted three times with diethylether; the organic phases were washed with water, dried with MgSO₄, filtered and evaporated to give 4.10 g of the title compound as yellow oil.

MS: 324.2 (M)⁺.

### G] [rac]-2-[4-(1-Carboxy-ethoxy)-2-methyl-phenoxy]-2-methyl-propionic acid ethyl ester

4.05 g (12.5 mmol) of [rac]-2-[4-(1-methoxycarbonyl-ethoxy)-2-methyl-phenoxy]-2-methyl-propionic acid ethyl ester were dissolved in 100 ml of THF and cooled down to a temperature of 2° C. 18.7 ml (18.7 mmol) of a LiOH-solution (1M in water) was added below 5° C. After 3 hours stirring between 2° C. and 5° C., the reaction mixture was poured into crashed ice and extracted three times with AcOEt; the organic phases were washed with water, dried with MgSO₄, filtered and evaporated to give 4.8 g of crude product which was purified by chromatography over silica gel with a gradient of MeCl₂ and MeOH to yield 2.53 g of the title compound as light yellow oil.

MS: 309.2 (M−H)⁻.

### Example 6

### [rac]-2-{4-[1-(Biphenyl-4-ylcarbamoyl)-ethoxy]-2-methyl-phenoxy}-2-methyl-propionic acid

In analogy to the procedures described in example 1A] and 1B], [rac]-2-[4-(1-carboxy-ethoxy)-2-methyl-phenoxy]-2-methyl-propionic acid ethyl ester (example 5G]) was reacted with 4-amino-biphenyl to give [rac]-2-{4-[1-(biphenyl-4-ylcarbamoyl)-ethoxy]-2-methyl-phenoxy}-2-methyl-propionic acid ethyl ester, which was subsequently saponified to yield the title compound as colorless solid.

MS: 432.2 (M−H)⁻.

### Example 7

### [rac]-2-(4-{1-[4-Cyclopropyl-2-(4-trifluoromethyl-phenyl)-pyrimidin-5-ylcarbamoyl]-ethoxy}-2-methyl-phenoxy)-2-methyl-propionic acid

In analogy to the procedures described in example 1A] and 1B], [rac]-2-[4-(1-carboxy-ethoxy)-2-methyl-phenoxy]-2-methyl-propionic acid ethyl ester (example 5G]) was reacted with 4-cyclopropyl-2-(4-trifluoromethyl-phenyl)-pyrimidin-5-ylamine (prepared from 4-trifluoromethyl-2-(4-trifluoromethyl-phenyl)-pyrimidine-5-carboxylic acid (example 1E]) in analogy to the procedures described in examples 5D] and 5E]) to give [rac]-2-(4-{1-[4-cyclopropyl-2-(4-trifluoromethyl-phenyl)-pyrimidin-5-ylcarbamoyl]-ethoxy}-2-methyl-phenoxy)-2-methyl-propionic acid ethyl ester, which was subsequently saponified to yield the title compound as yellow oil.

MS: 542.2 (M−H)⁻.

### Example 8

### [rac]-2-Methyl-2-{2-methyl-4-[1-(3′-trifluoromethyl-biphenyl-4-ylcarbamoyl)-ethoxy]-phenoxy}-propionic acid

A] In analogy to the procedures described in example 1A] and 1B], [rac]-2-[4-(1-carboxy-ethoxy)-2-methyl-phenoxy]-2-methyl-propionic acid ethyl ester (example 5G]) was reacted with 3′-trifluoromethyl-biphenyl-4-ylamine (example 8B]) to give [rac]-2-methyl-2-{2-methyl-4-[1-(3′-trifluoromethyl-biphenyl-4-ylcarbamoyl)-ethoxy]-phenoxy}-propionic acid ethyl ester, which was subsequently saponified to yield the title compound as colorless foam.

MS: 500.2 (M−H)⁻.

The necessary building block 3′-trifluoromethyl-biphenyl-4-ylamine used in the procedure above was prepared as follows:

### B] 3′-Trifluoromethyl-biphenyl-4-ylamine

3.0 g (13.3 mmol) of 4-iodoaniline was dissolved in 40 ml of 1,2-dimethoxyethane. To this solution were added 20 ml of water, 2.60 g (13.3 mmol) of 3-trifluoromethyl-phenylboronic acid, 2.50 g (22.9 mmol) of anhydrous sodium carbonate and 0.31 g (0.27 mmol) of tetrakis(triphenylphoshine)-palladium (0). This reaction mixture was stirred at 95° C. for 20 hours, then cooled down to RT, filtered and the residue was washed with AcOEt. The filtrate was then extracted twice with AcOEt; the organic phases were washed with water, dried with MgSO₄, filtered and evaporated to give 3.66 g of crude product which was purified by chromatography over silica gel with a gradient of n-heptane and AcOEt to yield 2.31 g of the title compound as a light brown solid.

MS: 237.8 (M+H)⁺.

### Example 9

### [rac]-2-Methyl-2-{2-methyl-4-[1-(4′-trifluoromethyl-biphenyl-3-ylcarbamoyl)-ethoxy)-phenoxy}-propionic acid

In analogy to the procedures described in example 1A] and 1B], [rac]-2-[4-(1-carboxy-ethoxy)-2-methyl-phenoxy]-2-methyl-propionic acid ethyl ester (example 5G]) was reacted with 4′-trifluoromethyl-biphenyl-3-ylamine (prepared in analogy to the procedure described in example 8B]) to give [rac]-2-methyl-2-{2-methyl-4-[1-(4′-trifluoromethyl-biphenyl-3-ylcarbamoyl)-ethoxy]-phenoxy}-propionic acid ethyl ester, which was subsequently saponified to yield the title compound as colorless foam.

MS: 500.2 (M−H)⁻.

### Example 10

### 2-Methyl-2-(2-methyl-4-{[2-methyl-6-(4-trifluoromethyl-phenyl)-pyridin-3-ylcarbamoyl]-methoxy}-phenoxy)-propionic acid

In analogy to the procedures described in example 1A] and 1B], 2-(4-carboxymethoxy-2-methyl-phenoxy)-2-methyl-propionic acid ethyl ester (prepared from 2-(4-hydroxy-2-methyl-phenoxy)-2-methyl-propionic acid ethyl ester (described in WO 02/092590) and methyl 2-chloro-acetate followed by saponification in analogy to the procedures described in examples 5F] and 5G]) was reacted with 2-methyl-6-(4-trifluoromethyl-phenyl)-pyridin-3-ylamine (example 5E]) to give 2-methyl-2-(2-methyl-4-{[2-methyl-6-(4-trifluoromethyl-phenyl)-pyridin-3-ylcarbamoyl]-methoxy}-phenoxy)-propionic acid ethyl ester, which was subsequently saponified to yield the title compound as colorless solid. MS: 501.2 (M−H)⁻.

### Example 11

### 2-[4-(Biphenyl-4-ylcarbamoylmethoxy)-2-methyl-phenoxy]-2-methyl-propionic acid

In analogy to the procedures described in example 1A] and 1B], 2-(4-carboxymethoxy-2-methyl-phenoxy)-2-methyl-propionic acid ethyl ester (prepared from 2-(4-hydroxy-2-methyl-phenoxy)-2-methyl-propionic acid ethyl ester (described in WO 02/092590) and methyl 2-chloro-acetate followed by saponification in analogy to the procedures described in examples 5F] and 5G]) was reacted with 4-amino-biphenyl to give 2-[4-(biphenyl-4-ylcarbamoylmethoxy)-2-methyl-phenoxy]-2-methyl-propionic acid ethyl ester, which was subsequently saponified to yield the title compound as colorless solid.

MS: 418.1 (M−H)⁻.

### Example 12

### 2-(4-{[4-Cyclopropyl-2-(4-trifluoromethyl-phenyl)-pyrimidin-5-ylcarbamoyl]-methoxy}-2-methyl-phenoxy)-2-methyl-propionic acid

In analogy to the procedures described in example 1A] and 1B], 2-(4-carboxymethoxy-2-methyl-phenoxy)-2-methyl-propionic acid ethyl ester (prepared from 2-(4-hydroxy-2-methyl-phenoxy)-2-methyl-propionic acid ethyl ester (described in WO 02/092590) and methyl 2-chloro-acetate followed by saponification in analogy to the procedures described in examples 5F] and 5G]) was reacted with 4-cyclopropyl-2-(4-trifluoromethyl-phenyl)-pyrimidin-5-ylamine (prepared from 4-trifluoromethyl-2-(4-trifluoromethyl-phenyl)-pyrimidine-5-carboxylic acid (example 1E]) in analogy to the procedures described in examples 5D] and 5E]) to give 2-(4-{[4-cyclopropyl-2-(4-trifluoromethyl-phenyl)-pyrimidin-5-ylcarbamoyl]-methoxy}-2-methyl-phenoxy)-2-methyl-propionic acid ethyl ester, which was subsequently saponified to yield the title compound as light yellow solid.

MS: 528.1(M−H)⁻.

### Example 13

### 2-Methyl-2-{2-methyl-4-[(3′-trifluoromethyl-biphenyl-4-ylcarbamoyl)-methoxy]-phenoxy}-propionic acid

In analogy to the procedures described in example 1A] and 1B], 2-(4-carboxymethoxy-2-methyl-phenoxy)-2-methyl-propionic acid ethyl ester (prepared from 2-(4-hydroxy-2-methyl-phenoxy)-2-methyl-propionic acid ethyl ester (described in WO 02/092590) and methyl 2-chloro-acetate followed by saponification in analogy to the procedures described in examples 5F] and 5G]) was reacted with 3′-trifluoromethyl-biphenyl-4-ylamine (example 8B]) to give 2-methyl-2-{2-methyl-4-[(3′-trifluoromethyl-biphenyl-4-ylcarbamoyl)-methoxy]-phenoxy}-propionic acid ethyl ester, which was subsequently saponified to yield the title compound as colorless solid.

MS: 486.3 (M−H)⁻.

### Example 14

### 2-Methyl-2-{2-methyl-4-[(4′-trifluoromethyl-biphenyl-3-ylcarbamoyl)-methoxy]-phenoxy}-propionic acid

In analogy to the procedures described in example 1A] and 1B], 2-(4-carboxymethoxy-2-methyl-phenoxy)-2-methyl-propionic acid ethyl ester (prepared from 2-(4-hydroxy-2-methyl-phenoxy)-2-methyl-propionic acid ethyl ester (described in WO 02/092590) and methyl 2-chloro-acetate followed by saponification in analogy to the procedures described in examples 5F] and 5G]) was reacted with 4′-trifluoromethyl-biphenyl-3-ylamine (prepared in analogy to the procedure described in example 8B]) to give 2-methyl-2-{2-methyl-4-[(4′-trifluoromethyl-biphenyl-3-ylcarbamoyl)-methoxy]-phenoxy}-propionic acid ethyl ester, which was subsequently saponified to yield the title compound as colorless solid.

MS: 486.3 (M−H)⁻.

### Example 15

### 2-Methyl-2-(4-{3-[2-methyl-6-(4-trifluoromethyl-phenyl)-pyridin-3-ylcarbamoyl]-propyl}-phenoxy)-propionic acid

### A] 2-Methyl-2-(4-{3-[2-methyl-6-(4-trifluoromethyl-phenyl)-pyridin-3-ylcarbamoyl]-propyl}-phenoxy)-propionic acid tert-butyl ester

In analogy to the procedure described in example 1A], 4-[4-(1-tert-butoxycarbonyl-1-methyl-ethoxy)-phenyl]-butyric acid (PCT Int. Appl. (2003), WO2003048130A2) was reacted with 2-methyl-6-(4-trifluoromethyl-phenyl)-pyridin-3-ylamine (example 5E]) to give the title compound as a light yellow solid.

MS: 557.5 (M+H)⁺.

### B] 2-Methyl-2-(4-{3-[2-methyl-6-(4-trifluoromethyl-phenyl)-pyridin-3-ylcarbamoyl]-propyl}-phenoxy)-propionic acid

0.16 g (0.29 mmol) of 2-methyl-2-(4-{3-[2-methyl-6-(4-trifluoromethyl-phenyl)-pyridin-3-ylcarbamoyl]-propyl}-phenoxy)-propionic acid tert-butyl ester was dissolved in 15 ml of MeCl₂. 0.09 ml=0.094 g (0.9 mmol) of anisole was added, followed by 0.22 ml=0.33 g (2.9 mmol) of trifluoroacetic acid. The reaction mixture was stirred at reflux (oil bath 50° C.) for 16 hours. The solvent was removed by evaporation and the residue dried in high vacuo for 2 hours. The crude product (0.24 g) was purified by flash chromatography (SiO₂, gradient of MeCl₂/MeOH) to give 0.137 g of the title compound as an off-white gum.

MS: 499.2 (M−H)⁻.

### Example 16

### 2-(4-{3-[4-Cyclopropyl-2-(4-trifluoromethyl-phenyl)-pyrimidin-5-ylcarbamoyl]-propyl}-phenoxy)-2-methyl-propionic acid

In analogy to the procedures described in example 1A] and 15B], 4-[4-(1-tert-butoxycarbonyl-1-methyl-ethoxy)-phenyl]-butyric acid (PCT Int. Appl. (2003), WO2003048130A2) was reacted with 4-cyclopropyl-2-(4-trifluoromethyl-phenyl)-pyrimidin-5-ylamine (prepared from 4-cyclopropyl-2-(4-trifluoromethyl-phenyl)-pyrimidine-5-carboxylic acid (example 1E]) in analogy to the procedures described in examples 5D] and 5E]) to give 2-(4-{3-[4-cyclopropyl-2-(4-trifluoromethyl-phenyl)-pyrimidin-5-ylcarbamoyl]-propyl}-phenoxy)-2-methyl-propionic acid tert-butyl ester, which was subsequently cleaved with trifluoroacetic acid to yield the title compound as an off-white foam.

MS: 526.1 (M−H)⁻.

### Example 17

### 2-Methyl-2-{4-[3-(3′-trifluoromethyl-biphenyl-4-ylcarbamoyl)-propyl]-phenoxy}-propionic acid

In analogy to the procedures described in example 1A] and 15B], 4-[4-(1-tert-butoxycarbonyl-1-methyl-ethoxy)-phenyl]-butyric acid (PCT Int. Appl. (2003), WO2003048130A2) was reacted with 3′-trifluoromethyl-biphenyl-4-ylamine (example 8B]) to give 2-methyl-2-{4-[3-(3′-trifluoromethyl-biphenyl-4-ylcarbamoyl)-propyl]-phenoxy}-propionic acid tert-butyl ester, which was subsequently cleaved with trifluoroacetic acid to yield the title compound as a light yellow oil.

MS: 484.3 (M−H)⁻.

### Example 18

### 2-Methyl-2-{4-[3-(4′-trifluoromethyl-biphenyl-3-ylcarbamoyl)-propyl]-phenoxy}-propionic acid

In analogy to the procedures described in example 1A] and 15B], 4-[4-(1-tert-butoxycarbonyl-1-methyl-ethoxy)-phenyl]-butyric acid (PCT Int. Appl. (2003), WO2003048130A2) was reacted with 4′-trifluoromethyl-biphenyl-3-ylamine (prepared in analogy to the procedure described in example 8B]) to give 2-methyl-2-{4-[3-(4′-trifluoromethyl-biphenyl-3-ylcarbamoyl)-propyl]-phenoxy}-propionic acid tert-butyl ester, which was subsequently cleaved with trifluoroacetic acid to yield the title compound as a light yellow foam.

MS: 484.3 (M−H)⁻.

### Example 19

### 2-Methyl-2-[2-methyl-4-(2-{[2-methyl-6-(4-trifluoromethyl-phenyl)-pyridine-3-carbonyl]-amino}-ethoxy)-phenoxy]-propionic acid

A] In analogy to the procedures described in example 1A] and 1B], 2-[4-(2-amino-ethoxy)-2-methyl-phenoxy]-2-methyl-propionic acid ethyl ester (example 19C]) was reacted with 2-methyl-6-(4-trifluoromethyl-phenyl)-nicotinic acid (example 5D]) to give 2-methyl-2-[2-methyl-4-(2-{[2-methyl-6-(4-trifluoromethyl-phenyl)-pyridine-3-carbonyl]-amino}-ethoxy)-phenoxy]-propionic acid ethyl ester, which was subsequently saponified to yield the title compound as colorless solid.

MS: 515.2 (M−H)⁻.

The necessary building block 2-[4-(2-amino-ethoxy)-2-methyl-phenoxy]-2-methyl-propionic acid ethyl ester used in the procedure above was prepared as follows:

### B] 2-[4-(2-tert-Butoxycarbonylamino-ethoxy)-2-methyl-phenoxy]-2-methyl-propionic acid ethyl ester

3.0 g (12.6 mmol) of 2-(4-hydroxy-2-methyl-phenoxy)-2-methyl-propionic acid ethyl ester (described in WO 02/092590), 2.24 ml=2.33 g (14.4 mmol) of N-Boc-ethanolamine and 4.43 g (16.9 mmol) of triphenylphosphine were dissolved in 120 ml of THF. The stirred reaction mixture was cooled down to 0° C. and a solution of 3.70 g (15.8 mmol) of di-tert-butyl azodicarboxylate in 30 ml of THF was added drop by drop. Then, the reaction mixture was warmed up to ambient temperature. After 20 hours, the solvent was evaporated and the residue (16.0 g) was purified by chromatography (SiO₂, heptane/AcOEt=95:5 to 4:1) to give 4.76 g of the title compound as colorless oil.

MS: 382.3 (M+H)⁺.

### C] 2-[4-(2-amino-ethoxy)-2-methyl-phenoxy]-2-methyl-propionic acid ethyl ester

1.60 g (4.2 mmol) of 2-[4-(2-tert-butoxycarbonylamino-ethoxy)-2-methyl-phenoxy]-2-methyl-propionic acid ethyl ester was dissolved in 20 ml of MeCl₂; 3.21 ml (42 mmol) of trifluoroacetic acid was added drop by drop. After two hours stirring at RT, the solvent was removed by evaporation, the residue was poured into crashed ice, the pH was adjusted to >9 with a saturated sodium carbonate solution (in water) and the mixture was extracted twice with AcOEt; the organic phases were washed with water, dried with MgSO₄, filtered and evaporated to give 1.2 g of crude product which was purified by chromatography over silica gel with a gradient of MeCl₂ and MeOH to yield 1.09 g of the title compound as colorless oil.

MS: 282.2 (M+H)⁺.

### Example 20

### 2-[4-(2-{[4-Cyclopropyl-2-(4-trifluoromethyl-phenyl)-pyrimidine-5-carbonyl]-amino}-ethoxy)-2-methyl-phenoxy]-2-methyl-propionic acid

In analogy to the procedures described in example 1A] and 1B], 2-[4-(2-amino-ethoxy)-2-methyl-phenoxy]-2-methyl-propionic acid ethyl ester (example 19C]) was reacted with 4-cyclopropyl-2-(4-trifluoromethyl-phenyl)-pyrimidine-5-carboxylic acid (example 1E]) to give 2-[4-(2-{[4-cyclopropyl-2-(4-trifluoromethyl-phenyl)-pyrimidine-5-carbonyl]-amino}-ethoxy)-2-methyl-phenoxy]-2-methyl-propionic acid ethyl ester, which was subsequently saponified to yield the title compound as colorless solid.

MS: 542.2 (M−H)⁻.

### Example 21

### 2-Methyl-2-[2-methyl-4-(2-{[4-trifluoromethyl-2-(4-trifluoromethyl-phenyl)-pyrimidine-5-carbonyl]-amino}-ethoxy)-phenoxy]-propionic acid

In analogy to the procedures described in example 1A] and 1B], 2-[4-(2-amino-ethoxy)-2-methyl-phenoxy]-2-methyl-propionic acid ethyl ester (example 19C]) was reacted with 4-trifluoromethyl-2-(4-trifluoromethyl-phenyl)-pyrimidine-5-carboxylic acid (prepared from ethyl 4,4,4-trifluoroacetoacetate: by i) treatment with triethyl orthoformate in analogy to the procedure described in example 1C] to yield 2-[1-ethoxy-meth-(E,Z)-ylidene]-4,4,4-trifluoro-3-oxo-butyric acid ethyl ester; ii) condensation with 4-(trifluoromethyl)benzamidine hydrochloride in analogy to the procedure described in example 1D] to give 4-trifluoromethyl-2-(4-trifluoromethyl-phenyl)-pyrimidine-5-carboxylic acid ethyl ester; iii) saponification in analogy to the procedure described in example 1E]) to give 2-methyl-2-[2-methyl-4-(2-{[4-trifluoromethyl-2-(4-trifluoromethyl-phenyl)-pyrimidine-5-carbonyl]-amino}-ethoxy)-phenoxy]-propionic acid ethyl ester, which was subsequently saponified to yield the title compound as colorless solid.

MS: 570.3 (M−H)⁻.

### Example 22

### 2-[4-(2-{[4-Methoxymethyl-2-(4-trifluoromethyl-phenyl)-pyrimidine-5-carbonyl]-amino}-ethoxy)-2-methyl-phenoxy]-2-methyl-propionic acid

In analogy to the procedures described in example 1A] and 1B], 2-[4-(2-amino-ethoxy)-2-methyl-phenoxy]-2-methyl-propionic acid ethyl ester (example 19C]) was reacted with 4-methoxymethyl-2-(4-trifluoromethyl-phenyl)-pyrimidine-5-carboxylic acid (prepared from 4-methoxy-3-oxo-butyric acid methyl ester: by i) treatment with triethyl orthoformate in analogy to the procedure described in example 1C] to yield 2-[1-ethoxy-meth-(E,Z)-ylidene]-4-methoxy-3-oxo-butyric acid methyl ester; ii) condensation with 4-(trifluoromethyl)benzamidine hydrochloride in analogy to the procedure described in example 1D] to give a mixture of 4-methoxymethyl-2-(4-trifluoromethyl-phenyl)-pyrimidine-5-carboxylic acid methyl and ethyl ester; iii) saponification in analogy to the procedure described in example 1E]) to give 2-[4-(2-{[4-methoxymethyl-2-(4-trifluoromethyl-phenyl)-pyrimidine-5-carbonyl]-amino}-ethoxy)-2-methyl-phenoxy]-2-methyl-propionic acid ethyl ester, which was subsequently saponified to yield the title compound as colorless oil.

MS: 546.2 (M−H)⁻.

### Example 23

### 2-[4-(2-{2-[4-Cyclopropyl-2-(4-trifluoromethyl-phenyl)-pyrimidin-5-yl]-acetylamino}-ethoxy)-2-methyl-phenoxy]-2-methyl-propionic acid

A] In analogy to the procedures described in example 1A] and 1B], 2-[4-(2-amino-ethoxy)-2-methyl-phenoxy]-2-methyl-propionic acid ethyl ester (example 19C]) was reacted with [4-cyclopropyl-2-(4-trifluoromethyl-phenyl)-pyrimidin-5-yl]-acetic acid (example 23E]) to give 2-[4-(2-{2-[4-cyclopropyl-2-(4-trifluoromethyl-phenyl)-pyrimidin-5-yl]-acetylamino}-ethoxy)-2-methyl-phenoxy]-2-methyl-propionic acid ethyl ester, which was subsequently saponified to yield the title compound as colorless solid.

MS: 556.2 (M−H)⁻.

The necessary building block [4-cyclopropyl-2-(4-trifluoromethyl-phenyl)-pyrimidin-5-yl]-acetic acid used in the procedure above was prepared as follows:

### B] [4-Cyclopropyl-2-(4-trifluoromethyl-phenyl)-pyrimidin-5-yl]-methanol

Within 10 min was dropped 31.6 ml (37.9 mmol) of 1.2 M DIBAL-H solution in toluene to a dry ice cooled (−50° C.) solution of 4.25 g (12.64 mmol) 4-cyclopropyl-2-(4-trifluoromethyl-phenyl)-pyrimidine-5-carboxylic acid ethyl ester (example 1D]) in 50 ml of THF. The reaction mixture was stirred 30 min at −50° C. and after letting rise the temperature to RT, the reaction was stirred for 1 h at RT. The reaction mixture was taken up in ether and washed with 1N HCL and water. The solvent was removed under reduced pressure to give 3.72 g of pure [4-cyclopropyl-2-(4-trifluoromethyl-phenyl)-pyrimidin-5-yl]-methanol.

MS: 295.1 (M+H)⁺.

### C] 5-Chloromethyl-4-cyclopropyl-2-(4-trifluoromethyl-phenyl)-pyrimidine

A mixture of 1.9 g (6.46 mmol) of [4-cyclopropyl-2-(4-trifluoromethyl-phenyl)-pyrimidin-5-yl]-methanol and 0.515 ml (7.1 mmol) thionylchloride in 20 ml dichloromethane was stirred for 1 h at RT. The reaction mixture was taken up in ether and washed with sodium bicarbonate solution and water. The ether phase was concentrated under reduced pressure to give 1.97 g of pure 5-chloromethyl-4-cyclopropyl-2-(4-trifluoromethyl-phenyl)-pyrimidine.

MS: 313.1 (M+H)⁺.

### D] [4-Cyclopropyl-2-(4-trifluoromethyl-phenyl)-pyrimidin-5-yl]-acetonitrile

3.12 g (10.0 mmol) of 5-chloromethyl-4-cyclopropyl-2-(4-trifluoromethyl-phenyl)-pyrimidine was dissolved in 7 ml of dimethyl sulfoxide; 0.59 g of sodium cyanide (12 mmol) was added and the mixture was stirred at 40° C. for 2 hours. Then, the reaction mixture was poured into a mixture of ice and water and the residue formed was filtered off. It was subsequently dissolved in tert-butyl methyl ether; the organic phase was washed with water, then with brine and dried over anhydrous sodium sulfate. During evaporation of the solvent, 1.0 g of the title compound separated as colorless solid. Another 1.1 g of the title compound could be obtained by chromatography (SiO₂) with dichlormethane as the eluent.

MS: 304.2 (M+H)⁺.

### E] [4-Cyclopropyl-2-(4-trifluoromethyl-phenyl)-pyrimidin-5-yl]-acetic acid

A mixture of 2.05 g (6.75 mmol) of the above prepared [4-cyclopropyl-2-(4-trifluoromethyl-phenyl)-pyrimidin-5-yl]-acetonitrile, 1.08 g of sodium hydroxide (27 mmol), 5 ml of water and 25 ml of propanol was stirred vigorously at 100° C. The hydrolysis was complete after 2 hours. The reaction mixture was then evaporated to dryness and the residue was dissolved in 20 ml of water; then, cold 4 N aqueous HCl was added and the compound was extracted with three portions of 25 ml of ethyl acetate; the combined organic phases were washed with water and brine, dried over anhydrous sodium sulfate and evaporated to dryness to yield after crystallization from ethyl acetate 1.56 g of the title product as colorless solid.

MS: 643.2 (2M−H)⁻.

### Example 24

### 2-Methyl-2-(2-methyl-4-{2-[(4′-trifluoromethyl-biphenyl-4-carbonyl)-amino]-ethoxy}-phenoxy)-propionic acid

In analogy to the procedures described in example 1A] and 1B], 2-[4-(2-amino-ethoxy)-2-methyl-phenoxy]-2-methyl-propionic acid ethyl ester (example 19C]) was reacted with 4′-trifluoromethyl-biphenyl-4-carboxylic acid (prepared in analogy to the procedure described in example 3B]) to give 2-methyl-2-(2-methyl-4-{2-[(4′-trifluoromethyl-biphenyl-4-carbonyl)-amino]-ethoxy}-phenoxy)-propionic acid ethyl ester, which was subsequently saponified to yield the title compound as colorless oil.

MS: 500.2 (M−H)⁻.

### Example 25

### 2-Methyl-2-(2-methyl-4-{2-[(3′-trifluoromethyl-biphenyl-4-carbonyl)-amino]-ethoxy}-phenoxy)-propionic acid

In analogy to the procedures described in example 1A] and 1B], 2-[4-(2-amino-ethoxy)-2-methyl-phenoxy]-2-methyl-propionic acid ethyl ester (example 19C]) was reacted with 3′-trifluoromethyl-biphenyl-4-carboxylic acid (example 3B]) to give 2-methyl-2-(2-methyl-4-{2-[(3′-trifluoromethyl-biphenyl-4-carbonyl)-amino]-ethoxy}-phenoxy)-propionic acid ethyl ester, which was subsequently saponified to yield the title compound as colorless oil.

MS: 500.2 (M−H)⁻.

### Example 26

### 2-[4-(2-{[4-Cyclopropyl-2-(3-trifluoromethyl-phenyl)-pyrimidine-5-carbonyl]-amino}-ethoxy)-2-methyl-phenoxy]-2-methyl-propionic acid

In analogy to the procedures described in example 1A] and 1B], 2-[4-(2-amino-ethoxy)-2-methyl-phenoxy]-2-methyl-propionic acid ethyl ester (example 19C]) was reacted with 4-cyclopropyl-2-(3-trifluoromethyl-phenyl)-pyrimidine-5-carboxylic acid-(example 2C]) to give 2-[4-(2-{[4-cyclopropyl-2-(3-trifluoromethyl-phenyl)-pyrimidine-5-carbonyl]-amino}-ethoxy)-2-methyl-phenoxy]-2-methyl-propionic acid ethyl ester, which was subsequently saponified to yield the title compound as colorless solid.

MS: 542.2 (M−H)⁻.

### Example 27

### [rac]-2-[4-(2-{[4-Cyclopropyl-2-(4-trifluoromethyl-phenyl)-pyrimidine-5-carbonyl]-amino}-1-methyl-ethoxy)-2-methyl-phenoxy]-2-methyl-propionic acid

In analogy to the procedures described in example 1A] and 1B], [rac]-2-[4-(2-amino-1-methyl-ethoxy)-2-methyl-phenoxy]-2-methyl-propionic acid ethyl ester (prepared from 2-(4-hydroxy-2-methyl-phenoxy)-2-methyl-propionic acid ethyl ester (WO 02/092590) and [rac]-(2-hydroxy-propyl)-carbamic acid tert-butyl ester [Bioorganic & Medicinal Chemistry (1998), 6(12), 2405-2419] in analogy to the procedures described in examples 19B] and 19C]) was reacted with 4-cyclopropyl-2-(4-trifluoromethyl-phenyl)-pyrimidine-5-carboxylic acid (example 1E]) to give [rac]-2-[4-(2-{[4-cyclopropyl-2-(4-trifluoromethyl-phenyl)-pyrimidine-5-carbonyl]-amino}-1-methyl-ethoxy)-2-methyl-phenoxy]-2-methyl-propionic acid ethyl ester, which was subsequently saponified to yield the title compound as colorless solid.

MS: 556.1 (M−H)⁻.

### Example 28

### [rac]-2-[4-(2-{[4-Cyclopropyl-2-(3-trifluoromethyl-phenyl)-pyrimidine-5-carbonyl]-amino}-1-methyl-ethoxy)-2-methyl-phenoxy]-2-methyl-propionic acid

In analogy to the procedures described in example 1A] and 1B], [rac]-2-[4-(2-amino-1-methyl-ethoxy)-2-methyl-phenoxy]-2-methyl-propionic acid ethyl ester (prepared from 2-(4-hydroxy-2-methyl-phenoxy)-2-methyl-propionic acid ethyl ester (WO 02/092590) and [rac]-(2-hydroxy-propyl)-carbamic acid tert-butyl ester [Bioorganic & Medicinal Chemistry (1998), 6(12), 2405-2419] in analogy to the procedures described in examples 19B] and 19C]) was reacted with 4-cyclopropyl-2-(3-trifluoromethyl-phenyl)-pyrimidine-5-carboxylic acid (example 2C]) to give [rac]-2-[4-(2-{[4-cyclopropyl-2-(3-trifluoromethyl-phenyl)-pyrimidine-5-carbonyl]-amino}-1-methyl-ethoxy)-2-methyl-phenoxy]-2-methyl-propionic acid ethyl ester, which was subsequently saponified to yield the title compound as colorless solid.

MS: 556.1 (M−H)⁻.

### Example 29

### [rac]-2-Methyl-2-(2-methyl-4-{1-methyl-2-[(3′-trifluoromethyl-biphenyl-4-carbonyl)-amino]-ethoxy}-phenoxy)-propionic acid

In analogy to the procedures described in example 1A] and 1B], [rac]-2-[4-(2-amino-1-methyl-ethoxy)-2-methyl-phenoxy]-2-methyl-propionic acid ethyl ester (prepared from 2-(4-hydroxy-2-methyl-phenoxy)-2-methyl-propionic acid ethyl ester (WO 02/092590) and [rac]-(2-hydroxy-propyl)-carbamic acid tert-butyl ester [Bioorganic & Medicinal Chemistry (1998), 6(12), 2405-2419] in analogy to the procedures described in examples 19B] and 19C]) was reacted with 3′-trifluoromethyl-biphenyl-4-carboxylic acid (example 3B]) to give [rac]-2-methyl-2-(2-methyl-4-{1-methyl-2-[(3′-trifluoromethyl-biphenyl-4-carbonyl)-amino]-ethoxy}-phenoxy)-propionic acid ethyl ester, which was subsequently saponified to yield the title compound as colorless solid.

MS: 514.2 (M−H)⁻.

### Example 30

### [rac]-2-Methyl-2-(2-methyl-4-{1-methyl-2-[(4′-trifluoromethyl-biphenyl-4-carbonyl)-amino]-ethoxy}-phenoxy)-propionic acid

In analogy to the procedures described in example 1A] and 1B], [rac]-2-[4-(2-amino-1-methyl-ethoxy)-2-methyl-phenoxy]-2-methyl-propionic acid ethyl ester (prepared from 2-(4-hydroxy-2-methyl-phenoxy)-2-methyl-propionic acid ethyl ester (WO 02/092590) and [rac]-(2-hydroxy-propyl)-carbamic acid tert-butyl ester [Bioorganic & Medicinal Chemistry (1998), 6(12), 2405-2419] in analogy to the procedures described in examples 19B] and 19C]) was reacted with 4′-trifluoromethyl-biphenyl-4-carboxylic acid (prepared in analogy to the procedure described in example 3B]) to give [rac]-2-methyl-2-(2-methyl-4-{1-methyl-2-[(4′-trifluoromethyl-biphenyl-4-carbonyl)-amino]-ethoxy}-phenoxy)-propionic acid ethyl ester, which was subsequently saponified to yield the title compound as colorless solid.

MS: 514.2 (M−H)⁻.

### Example 31

### [rac]-2-[4-(2-{[4-Cyclopropyl-2-(4-trifluoromethyl-phenyl)-pyrimidine-5-carbonyl]-amino}-propyl)-2-methyl-phenoxy]-2-methyl-propionic acid

A] In analogy to the procedures described in example 1A] and 1B], [rac]-2-[4-(2-amino-propyl)-2-methyl-phenoxy]-2-methyl-propionic acid ethyl ester (example 31E]) was reacted with 4-cyclopropyl-2-(4-trifluoromethyl-phenyl)-pyrimidine-5-carboxylic acid (example 1E]) to give [rac]-2-[4-(2-{[4-cyclopropyl-2-(4-trifluoromethyl-phenyl)-pyrimidine-5-carbonyl]-amino}-propyl)-2-methyl-phenoxy]-2-methyl-propionic acid ethyl ester, which was subsequently saponified to yield the title compound as colorless solid.

MS: 540.3 (M−H)⁻.

The necessary building block [rac]-2-[4-(2-amino-propyl)-2-methyl-phenoxy]-2-methyl-propionic acid ethyl ester used in the procedure above was prepared as follows:

### B] 2-Methyl-2-[2-methyl-4-(3-methyl-3-trimethylsilanyl-oxiranyl)-phenoxy]-propionic acid ethyl ester (mixture of diast.)

36.1 ml (47.0 mmol) of a sec-butyllithium solution (1.3M in cyclohexane) was diluted with 75 ml of THF and cooled down to −78° C. A solution of 8.30 ml (47.0 mmol) of (1-chloroethyl)-trimethylsilane in 30 ml of THF was added drop by drop, followed by 7.0 ml (47 mmol) of N,N,N,N-tetramethyl-ethylene-diamine; after stirring for 30 min. between −55° C. and −60° C., the reaction mixture was again cooled down to −78° C. and a solution of 7.38 g (29.5 mmol) 2-(4-formyl-2-methyl-phenoxy)-2-methyl-propionic acid ethyl ester [PCT Int. Appl. (2003), 98 pp. WO2004000762 A2] in 70 ml of THF was added and after another 30 min. at −78° C., it was warmed up to RT. The reaction mixture was then poured into crashed ice, the pH was adjusted to about 3 with HCl (1N) and it was then extracted twice with AcOEt; the organic phases were washed with water, dried with MgSO₄, filtered and evaporated to give 10.67 g of crude product which was purified by chromatography over silica gel with a gradient of n-heptane and AcOEt to yield 3.40 g of the title compound as colorless oil.

MS: 350.2 (M)⁺.

### C] 2-Methyl-2-[2-methyl-4-(2-oxo-propyl)-phenoxy]-propionic acid ethyl ester

3.20 g (9.1 mmol) of 2-methyl-2-[2-methyl-4-(3-methyl-3-trimethylsilanyl-oxiranyl)-phenoxy]-propionic acid ethyl ester (mixture of diast.) was dissolved in 30 ml of MeOH; then, 16 ml (32 mmol) of sulfuric acid (2 molar in water) was added at RT and after 30 min., the reaction mixture was poured into cold water and extracted twice with MeCl₂; the organic phases were washed with water, dried with MgSO₄, filtered and evaporated to give 2.66 g of crude product which was purified by chromatography over silica gel with a gradient of n-heptane and AcOEt to yield 2.06 g of the title compound as colorless oil.

MS: 278.2 (M)⁺.

### D] 2-(4-{2-[(E and/or Z)-Hydroxyimino]-propyl}-2-methyl-phenoxy)-2-methyl-propionic acid ethyl ester

2.0 g (7.2 mmol) of 2-methyl-2-[2-methyl-4-(2-oxo-propyl)-phenoxy)-propionic acid ethyl ester was dissolved in 20 ml of EtOH; 0.81 g (11.5 mmol) of hydroxylamine-hydrochloride was added, followed by a solution of 1.79 g (21.6 mmol) sodium acetate in 20 ml of water. After 2 hours, the solvents were removed by evaporation, the residue was dissolved in water and MeCl₂ and extracted twice with MeCl₂; the organic phases were washed with water, dried with MgSO₄, filtered and evaporated to give 2.24 g of crude product which was purified by chromatography over silica gel with a gradient of n-heptane and AcOEt to yield 1.80 g of the title compound as colorless oil.

MS: 293.2 (M)⁺.

### E] [rac]-2-[4-(2-Amino-propyl)-2-methyl-phenoxy]-2-methyl-propionic acid ethyl ester

1.56 g (5.3 mmol) of 2-(4-{2-[(E and/or Z)-hydroxyimino]-propyl}-2-methyl-phenoxy)-2-methyl-propionic acid ethyl ester was dissolved in 50 ml of glacial acetic acid; 0.3 g of platinum(IV)oxide was added and the well stirred mixture was hydrogenated at RT. After 1 hour, the catalyst was filtered off, washed with AcOH and the solvent evaporated. The residue was dissolved in water and MeCl₂, the pH was adjusted to >12 with NaOH (2N) and the mixture was extracted twice with MeCl₂; the organic phases were washed with water, dried with MgSO₄, filtered and evaporated to give 1.30 g of crude product which was purified by chromatography over silica gel with a gradient of MeCl₂ and MeOH to yield 1.14 g of the title compound as colorless oil.

MS: 280.1 (M+H)⁺.

### Example 32

### [rac]-2-[4-(2-{[4-Cyclopropyl-2-(3-trifluoromethyl-phenyl)-pyrimidine-5-carbonyl]-amino}-propyl)-2-methyl-phenoxy]-2-methyl-propionic acid

In analogy to the procedures described in example 1A] and 1B], [rac]-2-[4-(2-amino-propyl)-2-methyl-phenoxy]-2-methyl-propionic acid ethyl ester (example 31E]) was reacted with 4-cyclopropyl-2-(3-trifluoromethyl-phenyl)-pyrimidine-5-carboxylic acid (example 2C]) to give [rac]-2-[4-(2-{[4-cyclopropyl-2-(3-trifluoromethyl-phenyl)-pyrimidine-5-carbonyl]-amino}-propyl)-2-methyl-phenoxy]-2-methyl-propionic acid ethyl ester, which was subsequently saponified to yield the title compound as colorless solid.

MS: 540.3 (M−H)⁻.

### Example 33

### [rac]-2-Methyl-2-(2-methyl-4-{2-[(3′-trifluoromethyl-biphenyl-4-carbonyl)-amino]-propyl}-phenoxy)-propionic acid

In analogy to the procedures described in example 1A] and 1B], [rac]-2-[4-(2-amino-propyl)-2-methyl-phenoxy]-2-methyl-propionic acid ethyl ester (example 31E]) was reacted with 3′-trifluoromethyl-biphenyl-4-carboxylic acid (example 3B]) to give [rac]-2-methyl-2-(2-methyl-4-{2-[(3′-trifluoromethyl-biphenyl-4-carbonyl)-amino]-propyl}-phenoxy)-propionic acid ester, which was subsequently saponified to yield the title compound as colorless solid.

MS: 498.2 (M−H)⁻.

### Example 34

### [rac]-2-Methyl-2-(2-methyl-4-{2-[(4′-trifluoromethyl-biphenyl-4-carbonyl)-amino]-propyl}-phenoxy)-propionic acid

In analogy to the procedures described in example 1A] and 1B], [rac]-2-[4-(2-amino-propyl)-2-methyl-phenoxy]-2-methyl-propionic acid ethyl ester (example 31E]) was reacted with 4′-trifluoromethyl-biphenyl-4-carboxylic acid (prepared in analogy to the procedure described in example 3B]) to give [rac]-2-methyl-2-(2-methyl-4-{2-[(4′-trifluoromethyl-biphenyl-4-carbonyl)-amino]-propyl}-phenoxy)-propionic acid ethyl ester, which was subsequently saponified to yield the title compound as colorless solid.

MS: 498.1 (M−H)⁻.

### Example 35

### [rac]-2-[4-(1-{[4-Cyclopropyl-2-(4-trifluoromethyl-phenyl)-pyrimidine-5-carbonyl]-amino}-propyl)-2-methyl-phenoxy]-2-methyl-propionic acid

A] In analogy to the procedures described in example 1A] and 1B], [rac]-2-[4-(1-amino-propyl)-2-methyl-phenoxy]-2-methyl-propionic acid ethyl ester (example 35G]) was reacted with 4-cyclopropyl-2-(4-trifluoromethyl-phenyl)-pyrimidine-5-carboxylic acid (example 1E]) to give [rac]-2-[4-(1-{[4-cyclopropyl-2-(4-trifluoromethyl-phenyl)-pyrimidine-5-carbonyl]-amino}-propyl)-2-methyl-phenoxy]-2-methyl-propionic acid ethyl ester, which was subsequently saponified to yield the title compound as light yellow solid.

MS: 540.5 (M−H)⁻.

The necessary building block [rac]-2-[4-(1-amino-propyl)-2-methyl-phenoxy]-2-methyl-propionic acid ethyl ester used in the procedure above was prepared as follows:

### B] 3-Methyl-4-(2-trimethylsilanyl-ethoxymethoxy)-benzaldehyde

5.12 g (37.6 mmol) of 3-methyl-4-hydroxy-benzaldehyde was dissolved in 250 ml of MeCl₂; 19.7 ml=14.9 g (112.8 mmol) of N-ethyl-diisopropylamine was added at RT, then, 8.85 ml=8.36 g (45.1 mmol) of 2-(trimethylsilyl)ethoxymethyl chloride was added drop by drop below 25° C. After 5 hours, the reaction mixture was poured into crashed ice and the product was extracted twice with MeCl₂; the organic phases were washed with water, dried with MgSO₄, filtered and evaporated to give 11.59 g of crude product which was purified by chromatography over silica gel with a gradient of n-heptane and AcOEt to yield 10.61 g of the title compound as light yellow oil.

MS: 208.1 (M−C₃H₆O)⁺.

### C] [rac]-1-[3-Methyl-4-(2-trimethylsilanyl-ethoxymethoxy)-phenyl]-propan-1-ol

9.60 g (36.0 mmol) of 3-methyl-4-(2-trimethylsilanyl-ethoxymethoxy)-benzaldehyde was dissolved in 350 ml of THF and cooled down to −70° C.; to the stirred solution, 86.4 ml (43.2 mmol) of ethyl-lithium solution (0.5 molar in benzene) was added within 30 min. and then, the reaction mixture was warmed up to RT. After 5 hours, it was hydrolyzed by addition of 50 ml HCl (2N), then diluted with water and AcOEt and extracted twice with AcOEt; the organic phases were washed with water, dried with MgSO₄, filtered and evaporated to give 11.68 g of crude product which was purified by chromatography over silica gel with a gradient of n-heptane and AcOEt to yield 7.16 g of the title compound as light yellow oil.

MS: 296.2 (M)⁺.

### D] 1-[3-Methyl-4-(2-trimethylsilanyl-ethoxymethoxy)-phenyl]-propan-1-one

6.15 g (20.7 mmol) of [rac]-1-[3-methyl-4-(2-trimethylsilanyl-ethoxymethoxy)-phenyl]-propan-1-ol, 0.033 g (0.2 mmol) of TEMPO (2,2,6,6-tetramethyl-1-piperidinyloxy, free radical) and 0.135 g (0.4 mmol) of tetrabutylammonium bromide were dissolved in 150 ml of MeCl₂. After cooling down to 0° C., a solution of 6.14 g (24.9 mmol) of m-chloro-perbenzoic acid in 100 ml of MeCl₂ was added below 3° C. within 30 min.; the reaction was then warmed up to RT and after 16 hours, the solvent was removed by evaporation. The residue (11.32 g) was purified by chromatography over silica gel with a gradient of n-heptane and AcOEt to give 1.20 g of the title compound as yellow oil.

MS: 294.4 (M)⁺.

### E] 1-(4-Hydroxy-3-methyl-phenyl)-propan-1-one

1.17 g (4.0 mmol) of 1-[3-methyl-4-(2-trimethylsilanyl-ethoxymethoxy)-phenyl]-propan-1-one was dissolved in 30 ml of EtOH; while stirring, 1.86 ml (12 mmol) of a HCl-solution (6.4 molar in EtOH) was added and after 2 hours, the solvent was removed by evaporation and the residue was partitioned between water and MeCl₂ and extracted twice with MeCl₂; the organic phases were then washed with water, dried with MgSO₄, filtered and evaporated to give 0.73 g of crude product as a light brown solid.

MS: 165.4 (M+H)⁺.

### F] 2-Methyl-2-(2-methyl-4-propionyl-phenoxy)-propionic acid ethyl ester

0.71 g (4.3 mmol) of 1-(4-hydroxy-3-methyl-phenyl)-propan-1-one was dissolved in 30 ml of acetonitrile, 2.42 g (17.3 mmol) of potassium carbonate was added, followed by 1.99 ml=2.61 g (13 mmol) of ethyl 2-bromoisobutyrate. The reaction mixture was then heated at reflux for 7 hours. After cooling down to RT, the reaction mixture was poured into crashed ice and the product was extracted twice with AcOEt; the organic phases were washed with water, dried with MgSO₄, filtered and evaporated to give 1.057 g of crude product which was purified by chromatography over silica gel with a gradient of n-heptane and AcOEt to yield 0.848 g of the title compound as light yellow oil.

MS: 278.2 (M)⁺.

### G] [rac]-2-[4-(1-Amino-propyl)-2-methyl-phenoxy]-2-methyl-propionic acid ethyl ester

In analogy to the procedures described in example 31D] and 31E], 2-methyl-2-(2-methyl-4-propionyl-phenoxy)-propionic acid ethyl ester has been converted into 2-(4-{1-[(E and/or Z)-hydroxyimino]-propyl}-2-methyl-phenoxy)-2-methyl-propionic acid ethyl ester and subsequently hydrogenated to give the title compound as a light yellow solid.

MS: 263.2 (M−NH₃+H)⁺.

### Example 36

### [rac]-2-[4-(1-{[4-Cyclopropyl-2-(3-trifluoromethyl-phenyl)-pyrimidine-5-carbonyl]-amino}-propyl)-2-methyl-phenoxy]-2-methyl-propionic acid

In analogy to the procedures described in example 1A] and 1B], [rac]-2-[4-(1-amino-propyl)-2-methyl-phenoxy]-2-methyl-propionic acid ethyl ester (example 35G]) was reacted with 4-cyclopropyl-2-(3-trifluoromethyl-phenyl)-pyrimidine-5-carboxylic acid (example 2C]) to give [rac]-2-[4-(1-{[4-cyclopropyl-2-(3-trifluoromethyl-phenyl)-pyrimidine-5-carbonyl]-amino}-propyl)-2-methyl-phenoxy]-2-methyl-propionic acid ethyl ester, which was subsequently saponified to yield the title compound as light yellow amorphous solid.

MS: 540.4 (M−H)⁻.

### Example 37

### [rac]-2-Methyl-2-(2-methyl-4-{1-[(3′-trifluoromethyl-biphenyl-4-carbonyl)-amino]-propyl}-phenoxy)-propionic acid

In analogy to the procedures described in example 1A] and 1B], [rac]-2-[4-(1-amino-propyl)-2-methyl-phenoxy]-2-methyl-propionic acid ethyl ester (example 35G]) was reacted with 3′-trifluoromethyl-biphenyl-4-carboxylic acid (example 3B]) to give [rac]-2-methyl-2-(2-methyl-4-{1-[(3′-trifluoromethyl-biphenyl-4-carbonyl)-amino]-propyl}-phenoxy)-propionic acid ethyl ester, which was subsequently saponified to yield the title compound as light yellow solid.

MS: 498.1 (M−H)⁻.

### Example 38

### [rac]-2-Methyl-2-(2-methyl-4-{1-[(4′-trifluoromethyl-biphenyl-4-carbonyl)-amino]-propyl}-phenoxy)-propionic acid

In analogy to the procedures described in example 1A] and 1B], [rac]-2-[4-(1-amino-propyl)-2-methyl-phenoxy]-2-methyl-propionic acid ethyl ester (example 35G]) was reacted with 4′-trifluoromethyl-biphenyl-4-carboxylic acid (prepared in analogy to the procedure described in example 3B]) to give [rac]-2-methyl-2-(2-methyl-4-{1-[(4′-trifluoromethyl-biphenyl-4-carbonyl)-amino]-propyl}-phenoxy)-propionic acid ethyl ester, which was subsequently saponified to yield the title compound as light yellow solid.

MS: 498.1 (M−H)⁻.

### Example 39

### [rac]-2-Methyl-2-(2-methyl-4-{1-methyl-2-[2-methyl-6-(4-trifluoromethyl-phenyl)-pyridin-3-ylcarbamoyl]-ethyl}-phenoxy)-propionic acid

A] In analogy to the procedures described in example 1A] and 1B], [rac]-3-[4-(1-ethoxycarbonyl-1-methyl-ethoxy)-3-methyl-phenyl]-butyric acid (example 39D]) was reacted with 2-methyl-6-(4-trifluoromethyl-phenyl)-pyridin-3-ylamine (example 5E]) to give [rac]-2-methyl-2-(2-methyl-4-{1-methyl-2-[2-methyl-6-(4-trifluoromethyl-phenyl)-pyridin-3-ylcarbamoyl]-ethyl}-phenoxy)-propionic acid ethyl ester, which was subsequently saponified to yield the title compound as colorless solid.

MS: 513.3 (M−H)⁻.

The necessary building block [rac]-3-[4-(1-ethoxycarbonyl-1-methyl-ethoxy)-3-methyl-phenyl]-butyric acid used in the procedure above was prepared as follows:

### B] (E and/or Z)-3-[4-(1-Ethoxycarbonyl-1-methyl-ethoxy)-3-methyl-phenyl]-but-2-enoic acid ethyl ester

12.49 ml=13.99 g (60.5 mmol) of triethyl phosphonoacetate was diluted with 100 ml of dioxane under an argon atmosphere and cooled down to 10° C.; 1.98 g (45.4 mmol) of sodium hydride (55% dispersion in mineral oil) was then added in small portions. After 15 min., a solution of 4.0 g (15.1 mmol) of 2-(4-acetyl-2-methyl-phenoxy)-2-methyl-propionic acid ethyl ester [PCT Int. Appl. (2002), 35 pp. WO 2002096894A1] in 60 ml of dioxane was added and the mixture then stirred at reflux for 6 hours. After cooling down to RT, the reaction mixture was poured into crashed ice, the pH was adjusted to about 2 with HCl (2N) and it was then extracted twice with AcOEt; the organic phases were washed with water, dried with MgSO₄, filtered and evaporated to give 12.3 g of crude product which was purified by chromatography over silica gel with a gradient of n-heptane and AcOEt to yield 4.78 g of the title compound as colorless oil.

MS: 334.2 (M)⁺.

### C] [rac]-3-[4-(1-Ethoxycarbonyl-1-methyl-ethoxy)-3-methyl-phenyl]-butyric acid ethyl ester

4.70 g (14.1 mmol) of (E and/or Z)-3-[4-(1-ethoxycarbonyl-1-methyl-ethoxy)-3-methyl-phenyl]-but-2-enoic acid ethyl ester was dissolved in 150 ml of THF; 0.94 g of palladium (10% on activated carbon) was added and the well stirred mixture was hydrogenated at RT. After 1 hour, the catalyst was filtered off, washed with THF and the filtrate was evaporated to give 4.85 g of crude product which was purified by chromatography over silica gel with a gradient of n-heptane and AcOEt to yield 4.60 g of the title compound as colorless oil.

MS: 336.2 (M)⁺.

### D] [rac]-3-[4-(1-Ethoxycarbonyl-1-methyl-ethoxy)-3-methyl-phenyl]-butyric acid

1.50 g (4.5 mmol) of [rac]-3-[4-(1-ethoxycarbonyl-1-methyl-ethoxy)-3-methyl-phenyl]-butyric acid ethyl ester was dissolved in 50 ml of a mixture of THF/MeOH (7:3); 4.46 ml (4.46 mmol) of a LiOH-solution (1M in water) was added at RT and the mixture stirred for 8 hours. The reaction mixture was then poured into crashed ice, the pH was adjusted to about 2 with HCl (2N) and it was extracted twice with MeCl₂; the organic phases were washed with water, dried with MgSO₄, filtered and evaporated to give 1.40 g of crude product which was purified by chromatography over silica gel with a gradient of MeCl₂ and MeOH to yield 0.49 g of the title compound as colorless oil.

MS: 307.2 (M−H)⁻.

### Example 40

### [rac]-2-(4-{2-[4-Cyclopropyl-2-(4-trifluoromethyl-phenyl)-pyrimidin-5-ylcarbamoyl]-1-methyl-ethyl}-2-methyl-phenoxy)-2-methyl-propionic acid

In analogy to the procedures described in example 1A] and 1B], [rac]-3-[4-(1-ethoxycarbonyl-1-methyl-ethoxy)-3-methyl-phenyl]-butyric acid (example 39D]) was reacted with 4-cyclopropyl-2-(4-trifluoromethyl-phenyl)-pyrimidin-5-ylamine (prepared from 4-cyclopropyl-2-(4-trifluoromethyl-phenyl)-pyrimidine-5-carboxylic acid (example 1E]) in analogy to the procedures described in examples 5D] and 5E]) to give [rac]-2-(4-{2-[4-cyclopropyl-2-(4-trifluoromethyl-phenyl)-pyrimidin-5-ylcarbamoyl]-1-methyl-ethyl}-2-methyl-phenoxy)-2-methyl-propionic acid ethyl ester, which was subsequently saponified to yield the title compound as colorless solid.

MS: 540.4 (M−H)⁻.

### Example 41

### [rac]-2-Methyl-2-{2-methyl-4-[1-methyl-2-(4′-trifluoromethyl-biphenyl-4-ylcarbamoyl)-ethyl]-phenoxy}-propionic acid

In analogy to the procedures described in example 1A] and 1B], [rac]-3-[4-(1-ethoxycarbonyl-1-methyl-ethoxy)-3-methyl-phenyl]-butyric acid (example 39D]) was reacted with 4′-trifluoromethyl-biphenyl-4-ylamine (prepared in analogy to the procedure described in example 8B]) to give [rac]-2-methyl-2-{2-methyl-4-[1-methyl-2-(4′-trifluoromethyl-biphenyl-4-ylcarbamoyl)-ethyl]-phenoxy}-propionic acid ethyl ester, which was subsequently saponified to yield the title compound as colorless solid.

MS 498.2 (M−H)⁻.

### Example 42

### [rac]-2-Methyl-2-{2-methyl-4-[1-methyl-2-(3′-trifluoromethyl-biphenyl-4-ylcarbamoyl)-ethyl]-phenoxy}-propionic acid

In analogy to the procedures described in example 1A] and 1B], [rac]-3-[4-(1-ethoxycarbonyl-1-methyl-ethoxy)-3-methyl-phenyl]-butyric acid (example 39D]) was reacted with 3′-trifluoromethyl-biphenyl-4-ylamine (example 8B]) to give [rac]-2-methyl-2-{2-methyl-4-[1-methyl-2-(3′-trifluoromethyl-biphenyl-4-ylcarbamoyl)-ethyl]-phenoxy]-propionic acid ethyl ester, which was subsequently saponified to yield the title compound as colorless solid.

MS: 498.1 (M−H)⁻.

### Example 43

### [rac]-2-(4-{1-[4-Cyclopropyl-2-(4-trifluoromethyl-phenyl)-pyrimidin-5-ylcarbamoyl]-ethyl}-2-methyl-phenoxy)-2-methyl-propionic acid

A] In analogy to the procedures described in example 1A] and 1B], [rac]-2-[4-(1-carboxy-ethyl)-2-methyl-phenoxy]-2-methyl-propionic acid ethyl ester (example 43D]) was reacted with 4-cyclopropyl-2-(4-trifluoromethyl-phenyl)-pyrimidin-5-ylamine (prepared from 4-cyclopropyl-2-(4-trifluoromethyl-phenyl)-pyrimidine-5-carboxylic acid (example 1E]) in analogy to the procedures described in examples 5D] and 5E]) to give [rac]-2-(4-{1-[4-cyclopropyl-2-(4-trifluoromethyl-phenyl)-pyrimidin-5-ylcarbamoyl]-ethyl}-2-methyl-phenoxy)-2-methyl-propionic acid ethyl ester, which was subsequently saponified to yield the title compound as colorless solid.

MS: 526.1 (M−H)⁻.

The necessary building block [rac]-2-[4-(1-carboxy-ethyl)-2-methyl-phenoxy]-2-methyl-propionic acid ethyl ester used in the procedure above was prepared as follows:

### B] 2-[4-((E/Z)-2-Methoxy-1-methyl-vinyl)-2-methyl-phenoxy]-2-methyl-propionic acid ethyl ester

8.41 g (23.8 mmol) of (methoxymethyl)triphenylphosphonium chloride was suspended in 100 ml of THF; after cooling down to −20° C., 2.74 g (23.8 mmol) of potassium tert-butoxide was added in small portions. After 30 min., a solution of 5.20 g (19.7 mmol) of 2-(4-acetyl-2-methyl-phenoxy)-2-methyl-propionic acid ethyl ester [PCT Int. Appl. (2002), 35 pp. WO 2002096894A1] in 70 ml of THF was added drop by drop. After stirring for 1 hour at −20° C., the reaction mixture was warmed up slowly to RT. After stirring at RT for 20 hours, the mixture was poured into crashed ice and extracted twice with AcOEt; the organic phases were washed with water, dried with MgSO₄, filtered and evaporated to give 11.40 g of crude product which was purified by chromatography over silica gel with a gradient of n-heptane and AcOEt to yield 4.91 g of the title compound as colorless oil.

MS: 292.2 (M)⁺.

### C] [rac]-2-Methyl-2-[2-methyl-4-(1-methyl-2-oxo-ethyl)-phenoxy]-propionic acid ethyl ester

4.58 g (15.7 mmol) of 2-[4-((E/Z)-2-methoxy-1-methyl-vinyl)-2-methyl-phenoxy]-2-methyl-propionic acid ethyl ester was dissolved in 50 ml of THF; while stirring, 9,4 ml (18.8 mmol) of HCl (2.0N) was added and the reaction mixture heated up to reflux for 6 hours. Then, it was cooled down to 0° C., neutralized with sodium hydrogen carbonate solution and extracted twice with MeCl₂; the organic phases were washed with water, dried with MgSO₄, filtered and evaporated to give 3.89 g of crude product which was purified by chromatography over silica gel with a gradient of n-heptane and AcOEt to yield 3.13 g of the title compound as colorless oil.

MS: 278.2 (M)⁺.

### D] [rac]-2-[4-(1-Carboxy-ethyl)-2-methyl-phenoxy]-2-methyl-propionic acid ethyl ester

2.78 g (10.0 mmol) of [rac]-2-methyl-2-[2-methyl-4-(1-methyl-2-oxo-ethyl)-phenoxy]-propionic acid ethyl ester was dissolved in 40 ml of 2-methyl-2-propanol; 6.25 ml=4.12 g (50.0 mmol) of 2-methyl-2-butene was added and the reaction mixture was cooled down to 15° C. A solution of 3.46 g (26.0 mmol) of sodium chlorite and 2.38 g (15.0 mmol) of sodium dihydrogenphosphate-dihydrate in 25 ml of water was added drop by drop. After stirring for 20 hours at RT, the reaction mixture was poured into crashed ice and extracted twice with AcOEt; the organic phases were washed with water, dried with MgSO₄, filtered and evaporated to give 3.55 g of crude product which was purified by chromatography over silica gel with a gradient of n-heptane and AcOEt to yield 2.33 g of the title compound as light yellow oil.

MS: 293.2 (M−H)⁻.

### Example 44

### [rac]-2-Methyl-2-(2-methyl-4-{1-[2-methyl-6-(4-trifluoromethyl-phenyl)-pyridin-3-ylcarbamoyl]-ethyl}-phenoxy)-propionic acid

In analogy to the procedures described in example 1A] and 1B], [rac]-2-[4-(1-carboxy-ethyl)-2-methyl-phenoxy]-2-methyl-propionic acid ethyl ester (example 43D]) was reacted with 2-methyl-6-(4-trifluoromethyl-phenyl)-pyridin-3-ylamine (example 5E]) to give [rac]-2-methyl-2-(2-methyl-4-{1-[2-methyl-6-(4-trifluoromethyl-phenyl)-pyridin-3-ylcarbamoyl]-ethyl}-phenoxy)-propionic acid ethyl ester, which was subsequently saponified to yield the title compound as light yellow solid.

MS: 499.1 (M−H)⁻.

### Example 45

### [rac]-2-Methyl-2-{2-methyl-4-[1-(4′-trifluoromethyl-biphenyl-4-ylcarbamoyl)-ethyl]-phenoxy}-propionic acid

In analogy to the procedures described in example 1A] and 1B], [rac]-2-[4-(1-carboxy-ethyl)-2-methyl-phenoxy]-2-methyl-propionic acid ethyl ester (example 43D]) was reacted with 4′-trifluoromethyl-biphenyl-4-ylamine (prepared in analogy to the procedure described in example 8B]) to give [rac]-2-methyl-2-{2-methyl-4-[1-(4′-trifluoromethyl-biphenyl-4-ylcarbamoyl)-ethyl]-phenoxy}-propionic acid ethyl ester, which was subsequently saponified to yield the title compound as light yellow solid.

MS: 484.3 (M−H)⁻.

### Example 46

### [rac]-2-Methyl-2-{2-methyl-4-[1-(3′-trifluoromethyl-biphenyl-4-ylcarbamoyl)-ethyl]-phenoxy}-propionic acid

In analogy to the procedures described in example 1A] and 1B], [rac]-2-[4-(1-carboxy-ethyl)-2-methyl-phenoxy]-2-methyl-propionic acid ethyl ester (example 43D]) was reacted with 3′-trifluoromethyl-biphenyl-4-ylamine (example 8B]) to give [rac]-2-methyl-2-{2-methyl-4-[1-(3′-trifluoromethyl-biphenyl-4-ylcarbamoyl)-ethyl]-phenoxy}-propionic acid ethyl ester, which was subsequently saponified to yield the title compound as light yellow foam.

MS: 484.3 (M−H)⁻.

### Example 47

### [rac]-2-Methyl-2-[2-methyl-4-(1-{[2-(4-trifluoromethoxy-phenyl)-4-trifluoromethyl-pyrimidine-5-carbonyl]-amino}-ethyl)-phenoxy]-propionic acid

A] In analogy to the procedures described in example 1A] and 1B], [rac]-2-[4-(1-amino-ethyl)-2-methyl-phenoxy]-2-methyl-propionic acid ethyl ester [PCT Int. Appl. (2002), 35 pp. WO 2002096894A1] was reacted with 2-(4-trifluoromethoxy-phenyl)-4-trifluoromethyl-pyrimidine-5-carboxylic acid (example 47C]) to give [rac]-2-methyl-2-[2-methyl-4-(1-{[2-(4-trifluoromethoxy-phenyl)-4-trifluoromethyl-pyrimidine-5-carbonyl]-amino}-ethyl)-phenoxy]-propionic acid ethyl ester, which was subsequently saponified to yield the title compound as colorless oil.

MS: 570.5 (M−H)⁻.

The necessary building block 2-(4-trifluoromethoxy-phenyl)-4-trifluoromethyl-pyrimidine-5-carboxylic acid used in the procedure above was prepared as follows:

### B] 2-(4-Trifluoromethoxy-phenyl)-4-trifluoromethyl-pyrimidine-5-carboxylic acid ethyl ester

A solution of 0.21 g (0.30 mmol) bis(triphenylphosphine)palladium(II)chloride, 2.55 g (10 mmol) ethyl 2-chloro-4-(trifluoromethyl)pyrimidine-5-carboxylate and 2.68 g (13 mmol) of 4-(trifluoromethoxy)phenylboronic acid in 50 ml degassed toluene was treated with 10 ml aqueous 2M K₃PO₄ solution and heated at 80° C. for 20 h. The reaction was cooled to RT and extracted with saturated aqueous NaCl (0° C.)/Et₂O (3×). The organic phases were washed with H₂O, aqueous 10% NaCl, dried (Na₂SO₄) and evaporated. Purification by flash-chromatography on silica gel (heptane/ether 98:2 to 96:4) gave 1.78 g of the title compound as an off-white powder.

MS: 379.9 (M)⁺.

### C] 2-(4-Trifluoromethoxy-phenyl)-4-trifluoromethyl-pyrimidine-5-carboxylic acid

In analogy to the procedure described in example 1E], saponification of 2-(4-trifluoromethoxy-phenyl)-4-trifluoromethyl-pyrimidine-5-carboxylic acid ethyl ester gave the title compound as a white powder.

MS: 351.1 (M−H)⁻.

### Example 48

Film coated tablets containing the following ingredients can be manufactured in a conventional manner:

| | |
|---|---|
| Ingredients | Per tablet |

| | | | | |
|---|---|---|---|---|
| Kernel: | | | | |
| Compound of formula (I) | 10.0 | mg | 200.0 | mg |
| Microcrystalline cellulose | 23.5 | mg | 43.5 | mg |
| Lactose hydrous | 60.0 | mg | 70.0 | mg |
| Povidone K30 | 12.5 | mg | 15.0 | mg |
| Sodium starch glycolate | 12.5 | mg | 17.0 | mg |
| Magnesium stearate | 1.5 | mg | 4.5 | mg |
| (Kernel Weight) | 120.0 | mg | 350.0 | mg |
| Film Coat: | | | | |
| Hydroxypropyl methyl cellulose | 3.5 | mg | 7.0 | mg |
| Polyethylene glycol 6000 | 0.8 | mg | 1.6 | mg |
| Talc | 1.3 | mg | 2.6 | mg |
| Iron oxide (yellow) | 0.8 | mg | 1.6 | mg |
| Titanium dioxide | 0.8 | mg | 1.6 | mg |

The active ingredient is sieved and mixed with microcrystalline cellulose and the mixture is granulated with a solution of polyvinylpyrrolidone in water. The granulate is mixed with sodium starch glycolate and magnesium stearate and compressed to yield kernels of 120 or 350 mg respectively. The kernels are lacquered with an aqueous solution/suspension of the above mentioned film coat.

### Example 49

Capsules containing the following ingredients can be manufactured in a conventional manner:

| | |
|---|---|
| Ingredients | Per capsule |

| | | |
|---|---|---|
| Compound of formula (I) | 25.0 | mg |
| Lactose | 150.0 | mg |
| Maize starch | 20.0 | mg |
| Talc | 5.0 | mg |

The components are sieved and mixed and filled into capsules of size 2.

### Example 50

Injection solutions can have the following composition:

| Compound of formula (I) | 3.0 | mg |
|---|---|---|
| Gelatine | 150.0 | mg |
| Phenol | 4.7 | mg |
| Sodium carbonate | to obtain a final pH of 7 | |
| Water for injection solutions | ad 1.0 | ml |

### Example 51

Soft gelatin capsules containing the following ingredients can be manufactured in a conventional manner:

| Capsule contents | | |
|---|---|---|
| Compound of formula (I) | 5.0 | mg |
| Yellow wax | 8.0 | mg |
| Hydrogenated Soya bean oil | 8.0 | mg |
| Partially hydrogenated plant oils | 34.0 | mg |
| Soya bean oil | 110.0 | mg |
| Weight of capsule contents | 165.0 | mg |
| Gelatin capsule | | |
| Gelatin | 75.0 | mg |
| Glycerol 85% | 32.0 | mg |
| Karion 83 | 8.0 mg (dry matter) | |
| Titanium dioxide | 0.4 | mg |
| Iron oxide yellow | 1.1 | mg |

The active ingredient is dissolved in a warm melting of the other ingredients and the mixture is filled into soft gelatin capsules of appropriate size. The filled soft gelatin capsules are treated according to the usual procedures.

### Example 52

Sachets containing the following ingredients can be manufactured in a conventional manner:

| | | |
|---|---|---|
| Compound of formula (I) | 50.0 | mg |
| Lactose, fine powder | 1015.0 | mg |
| Microcrystalline cellulose (AVICEL PH 102) | 1400.0 | mg |
| Sodium carboxymethyl cellulose | 14.0 | mg |
| Polyvinylpyrrolidone K 30 | 10.0 | mg |
| Magnesium stearate | 10.0 | mg |
| Flavoring additives | 1.0 | mg |

The active ingredient is mixed with lactose, microcrystalline cellulose and sodium carboxymethyl cellulose and granulated with a mixture of polyvinylpyrrolidone in water. The granulate is mixed with magnesium stearate and the flavoring additives and filled into sachets.

It is to be understood that the invention is not limited to the particular embodiments of the invention described above, as variations of the particular embodiments may be made and still fall within the scope of the appended claims.

What is claimed is:

1. A compound of the formula (I): 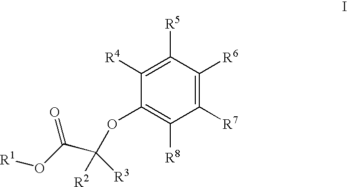
wherein:
R¹ is hydrogen or C₁₋₇-alkyl;
R¹ and R³ independently from each other are hydrogen or C₁₋₇-alkyl,
R⁴ and R⁸ independently from each other are selected from the group consisting of hydrogen, C₁₋₇-alkyl, C₃₋₇-cycloalkyl, halogen, C₁₋₇-alkoxy-C₁₋₇-alkyl,
C₂₋₇-alkenyl, C₂₋₇-alkinyl, fluoro-C₁₋₇-alkyl, cyano-C₁₋₇-alkyl and cyano;
R⁵, R⁶ and R⁷ independently from each other are selected from the group consisting of hydrogen, C₁₋₇-alkyl, C₃₋₇-cycloalkyl, halogen, C₁₋₇-alkoxy-C₁₋₇-alkyl,
C₂₋₇-alkenyl, C₂₋₇-alkinyl, fluoro-C₁₋₇-alkyl, cyano-C₁₋₇-alkyl and cyano;
and one of R⁵, R⁶ and R⁷ is 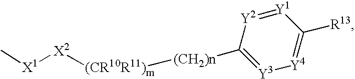
wherein
X¹ is selected from the group consisting of
—(CR¹⁴R¹⁵), —(CR¹⁴R¹⁵)CH₂—, —CH₂(CR¹⁴R¹⁵)—, —CH₂CH₂CH₂—,
—(CR¹⁴R¹⁵)CH₂CH₂—, —CH₂(CR¹⁴R¹⁵)CH₂—, —CH₂CH₂(CR¹⁴R¹⁵)—,
—CH₂CH₂CH₂CH₂—, —(CR¹⁴R¹⁵)CH₂CH₂CH₂—, —CH₂(CR¹⁴R¹⁵)CH₂CH₂—,
—CH₂CH₂(CR¹⁴R¹⁵)CH₂—, and —CH₂CH₂CH₂(CR¹⁴R¹⁵)—,
or, in addition,
X¹ is selected from the group consisting of
—OCH₂—, —O(CR¹⁴R¹⁵)—, —OCH₂CH₂—, —O(CR¹⁴H)CH₂—,
—OCH₂(CR¹⁴R¹⁵)—, —OCH₂CH₂CH₂—, —O(CR¹⁴H)CH₂CH₂—,
—OCH₂(CR¹⁴R¹⁵)CH₂—, and —OCH₂CH₂(CR¹⁴R¹⁵)—,
when X² is —CONR⁹—; or
X¹ is selected from the group consisting of
—OCH₂CH₂—, —O(CR¹⁴H)CH₂—, —OCH₂(CR¹⁴R¹⁵)—,
—OCH₂CH₂CH₂—, —O(CR¹⁴H)CH₂CH₂—, —OCH₂(CR¹⁴R¹⁵)CH₂—, and
—OCH₂CH₂(CR¹⁴R¹⁵)—, when X² is —NR⁹CO—,
X² is —NR⁹CO— or —CONR⁹—;
R⁹ is selected from the group consisting of hydrogen, C₁₋₇-alkyl, C₃₋₇-cycloalkyl, fluoro-C₁₋₇-alkyl, hydroxy-C₂₋₇-alkyl, and C₁₋₇-alkoxy-C₂₋₇-alkyl;
Y¹, Y², Y³ and Y⁴ are N or C—R¹², whereas none, one or two of Y¹, Y², Y³ and Y⁴ are N and the other ones are C—R¹²;
R¹⁰ is selected from the group consisting of C₁₋₇-alkyl, C₃₋₇-cycloalkyl, fluoro-C₁₋₇-alkyl, and C₁₋₇-alkoxy-C₁₋₇-alkyl;
R¹¹ is selected from the group consisting of hydrogen, C₁₋₇-alkyl, and
C₁₋₇-alkoxy-C₁₋₇-alkyl;
R¹² independently from each other in each occurrence is selected from the group consisting of hydrogen, C₁₋₇-alkyl, C₃₋₇-cycloalkyl, fluoro-C₁₋₇-alkyl, C₁₋₇-alkoxy-C₁₋₇-alkyl, hydroxy-C₁₋₇-alkyl, C₁₋₇-alkylthio-C₁₋₇-alkyl, carboxy-C₁₋₇-alkoxy-C₁₋₇-alkyl, carboxy, carboxy-C₁₋₇-alkyl, mono- or di-C₁₋₇-alkyl-amino-C₁₋₇-alkyl,
C₁₋₇-alkanoyl-C₁₋₇-alkyl, C₂₋₇-alkenyl, and C₂₋₇-alkinyl;
R¹³ is aryl or heteroaryl;
R¹⁴ is selected from the group consisting of C₁₋₇-alkyl, C₃₋₇-cycloalkyl, fluoro-C₁₋₇-alkyl, and C₁₋₇-alkoxy-C₁₋₇-alkyl;
R¹⁵ is selected from the group consisting of hydrogen, C₁₋₇-alkyl, C₃₋₇-cycloalkyl, fluoro-C₁₋₇-alkyl, and C₁₋₇-alkoxy-C₁₋₇-alkyl;
m is 0 or 1;
n is 0, 1, 2 or 3; and
all pharmaceutically acceptable salts and/or esters thereof.

2. The compound according to claim 1, wherein one or two of Y¹, Y², Y³ and Y⁴ are N and the other ones are C—R¹² and R¹² independently from each other in each occurrence is selected from the group consisting of hydrogen, C₁₋₇-alkyl, C₃₋₇-cycloalkyl, fluoro-C₁₋₇-alkyl and C₁₋₇-alkoxy-C₁₋₇-alkyl.

3. The compound according to claim 1, wherein Y¹ and Y⁴ are N, Y² and Y³ are C—R¹² and R¹² independently from each other in each occurrence is selected from the group consisting of hydrogen, C₁₋₇-alkyl, C₃₋₇-cycloalkyl, fluoro-C₁₋₇-alkyl and C₁₋₇-alkoxy-C₁₋₇-alkyl.

4. The compound according to claim 1, wherein
X² is —NR⁹CO—;
X¹ is selected from the group consisting of
—(CR¹⁴R¹⁵), —(CR¹⁴R¹⁵)CH₂—, —CH₂(CR¹⁴R¹⁵)—, —CH₂CH₂CH₂—,
—(CR¹⁴R¹⁵)CH₂CH₂—, —CH₂(CR¹⁴R¹⁵)CH₂—, —CH₂CH₂(CR⁴R⁵)—,
—CH₂CH₂CH₂CH₂—, —(CR¹⁴R¹⁵)CH₂CH₂CH₂—, —CH₂(CR¹⁴R¹⁵)CH₂CH₂—,
—CH₂CH₂(CR¹⁴R¹⁵)CH₂—, —CH₂CH₂CH₂(CR¹⁴R¹⁵)—,
—OCH₂CH₂—, —O(CR¹⁴H)CH₂—, —OCH₂(CR¹⁴R¹⁵)—,
—OCH₂CH₂CH₂—, —O(CR¹⁴H)CH₂CH₂—, —OCH₂(CR¹⁴R¹⁵)CH₂—, and
—OCH₂CH₂(CR¹⁴R¹⁵)—;
R⁹ is selected from the group consisting of hydrogen, C, ₇-allyl, C₃₋₇-cycloalkyl, fluoro-C₁₋₇-alkyl, hydroxy-C₂₋₇-alkyl, and C₁₋₇-alkoxy-C₂₋₇-alkyl;
R¹⁴ is selected from the group consisting of C₁₋₇-alkyl, C₃₋₇-cycloalkyl, fluoro-C₁₋₇-alkyl, and C₁₋₇-alkoxy-C₁₋₇-alkyl; and
R¹⁵ is selected from the group consisting of hydrogen, C₁₋₇-alkyl, C₃₋₇-cycloalkyl, fluoro-C₁₋₇-alkyl, and C₁₋₇-alkoxy-C₁₋₇-alkyl.

5. The compound according to claim 1, wherein
X² is —CONR⁹—;
X¹ is selected from the group consisting of
—(CR¹⁴R¹⁵), —(CR¹⁴R¹⁵)CH₂—, —CH₂(CR¹⁴R¹⁵)—, —CH₂CH₂CH₂—,
—(CR¹⁴R¹⁵)CH₂CH₂—, —CH₂(CR¹⁴R¹⁵)CH₂—, —CH₂CH₂(CR¹⁴R¹⁵)—,
—CH₂CH₂CH₂CH₂—, —(CR¹⁴R¹⁵)CH₂CH₂CH₂—, —CH₂(CR¹⁴R¹⁵)CH₂CH₂—,
—CH₂CH₂(CR¹⁴R¹⁵)CH₂—, —CH₂CH₂CH₂(CR¹⁴R¹⁵)—,
—OCH₂—, —O(CR¹⁴R¹⁵)—, —OCH₂CH₂—, —O(CR¹⁴H)CH₂—,
—OCH₂(CR¹⁴R¹⁵)—, —OCH₂CH₂CH₂—, —O(CR¹⁴H)CH₂CH₂—,
—OCH₂(CR¹⁴R¹⁵)CH₂—, and —OCH₂CH₂(CR¹⁴R¹⁵)—;
R⁹ is selected from the group consisting of hydrogen, C₁₋₇-alkyl, C₃₋₇-cycloalkyl, fluoro-C₁₋₇-alkyl, hydroxy-C₂₋₇-alkyl, and C₁₋₇-alkoxy-C₂₋₇-alkyl;
R¹⁴ is selected from the group consisting of C₁₋₇-alkyl, C₃₋₇-cycloalkyl, fluoro-C₁₋₇-alkyl, and C₁₋₇-alkoxy-C₁₋₇-alkyl; and
R¹⁵ is selected from the group consisting of hydrogen, C₁₋₇-alkyl, C₃₋₇-cycloalkyl, fluoro-C₁₋₇-alkyl, and C₁₋₇-alkoxy-C₁₋₇-alkyl.

6. The compound according to claim 1, wherein R⁹ is hydrogen.

7. The compound according to claim 1, wherein
X¹ is selected from the group consisting of
—(CR¹⁴R¹⁵), —(CR¹⁴R¹⁵)CH₂—, —CH₂(CR¹⁴R¹⁵)—, —CH₂CH₂CH₂—,
—(CR¹⁴R¹⁵)CH₂CH₂—, —CH₂(CR¹⁴R¹⁵)CH₂—, —CH₂CH₂(CR¹⁴R¹⁵)—,
—CH₂CH₂CH₂CH₂—, —(CR¹⁴R¹⁵)CH₂CH₂CH₂—, —CH₂(CR¹⁴R¹⁵)CH₂CH₂—,
—CH₂CH₂(CR¹⁴R¹⁵)CH₂—, and —CH₂CH₂CH₂(CR¹⁴R¹⁵)—;
R¹⁴ is C₁₋₇-alkyl and R¹⁵ is hydrogen.

8. The compound according to claim 1, wherein Y¹, Y², Y³ and Y⁴ are C—R¹² and R¹² independently from each other in each occurrence is selected from the group consisting of hydrogen, C₁₋₇-alkyl, C₃₋₇-cycloalkyl, fluoro-C₁₋₇-alkyl and C₁₋₇-alkoxy-C₁₋₇-alkyl.

9. The compound according to claim 1, wherein R⁶ is 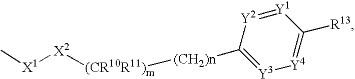
and R⁴, R⁵, R⁷ and R⁸ independently from each other are selected from hydrogen or C₁₋₇-alkyl.

10. The compound according to claim 1, wherein R⁵ or R⁷ is

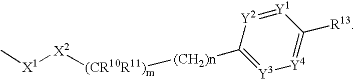

11. The compound according to claim 1, wherein R¹ is hydrogen.

12. The compound according to claim 1, wherein R² and R³ are methyl.

13. The compound according to claim 1, wherein m is 0.

14. The compound according to claim 1, wherein n is 0.

15. The compound according to claim 1, wherein n is 1.

16. The compound according to claim 1, wherein R¹³ is unsubstituted phenyl or phenyl substituted with one to three groups selected from C₁₋₇-alkyl, C₁₋₇-alkoxy, halogen, fluoro-C₁₋₇-alkyl, fluoro-C₁₋₇-alkoxy and cyano.

17. The compound according to claim 1, wherein R¹³ is phenyl substituted with halogen, fluoro-C₁₋₇-alkyl or fluoro-C₁₋₇-alkoxy.

18. The compound according to claim 1, selected from the group consisting of
[rac]-2-[4-(1-{[4-cyclopropyl-2-(4-trifluoromethyl-phenyl)-pyrimidine-5-carbonyl]-amino}-ethyl)-2-methyl-phenoxy]-2-methyl-propionic acid,
[rac]-2-[4-(1-{[4-cyclopropyl-2-(3-trifluoromethyl-phenyl)-pyrimidine-5-carbonyl]-amino}-ethyl)-2-methyl-phenoxy]-2-methyl-propionic acid,
[rac]-2-methyl-2-(2-methyl-4-{1-[(3′-trifluoromethyl-biphenyl-4-carbonyl)-amino]-ethyl}-phenoxy)-propionic acid,
[rac]-2-methyl-2-(2-methyl-4-{1-[(4′-trifluoromethyl-biphenyl-4-carbonyl)-amino]-ethyl}-phenoxy)-propionic acid,
[rac]-2-methyl-2-(2-methyl-4-{1-[2-methyl-6-(4-trifluoromethyl-phenyl)-pyridin-3-ylcarbamoyl]-ethoxy}-phenoxy)-propionic acid,
[rac]-2-{4-[1-(biphenyl-4-ylcarbamoyl)-ethoxy]-2-methyl-phenoxy}-2-methyl-propionic acid,
[rac]-2-(4-{1-[4-cyclopropyl-2-(4-trifluoromethyl-phenyl)-pyrimidin-5-ylcarbamoyl]-ethoxy}-2-methyl-phenoxy)-2-methyl-propionic acid,
[rac]-2-methyl-2-{2-methyl-4-[1-(3′-trifluoromethyl-biphenyl-4-ylcarbamoyl)-ethoxy]-phenoxy}-propionic acid,
[rac]-2-methyl-2-{2-methyl-4-[1-(4′-trifluoromethyl-biphenyl-3-ylcarbamoyl)-ethoxy]-phenoxy}-propionic acid,
2-methyl-2-(2-methyl-4-{[2-methyl-6-(4-trifluoromethyl-phenyl)-pyridin-3-ylcarbamoyl]-methoxy}-phenoxy)-propionic acid,
2-[4-(biphenyl-4-ylcarbamoylmethoxy)-2-methyl-phenoxy]-2-methyl-propionic acid,
2-(4-{[4-cyclopropyl-2-(4-trifluoromethyl-phenyl)-pyrimidin-5-ylcarbamoyl]-methoxy}-2-methyl-phenoxy)-2-methyl-propionic acid,
2-methyl-2-{2-methyl-4-[(3′-trifluoromethyl-biphenyl-4-ylcarbamoyl)-methoxy]-phenoxy}-propionic acid,
2-methyl-2-{2-methyl-4-[(4′-trifluoromethyl-biphenyl-3-ylcarbamoyl)-methoxy]-phenoxy}-propionic acid,
2-methyl-2-(4-{3-[2-methyl-6-(4-trifluoromethyl-phenyl)-pyridin-3-ylcarbamoyl]-propyl}-phenoxy)-propionic acid,
2-(4-{3-[4-cyclopropyl-2-(4-trifluoromethyl-phenyl)-pyrimidin-5-ylcarbamoyl]-propyl}-phenoxy)-2-methyl-propionic acid,
2-methyl-2-{4-[3-(3′-trifluoromethyl-biphenyl-4-ylcarbamoyl)-propyl]-phenoxy}-propionic acid,
2-methyl-2-[4-[3-(4′-trifluoromethyl-biphenyl-3-ylcarbamoyl)-propyl]-phenoxy}-propionic acid,
2-methyl-2-[2-methyl-4-(2-{[2-methyl-6-(4-trifluoromethyl-phenyl)-pyridine-3-carbonyl]-amino}-ethoxy)-phenoxy]-propionic acid,
2-[4-(2-{[4-cyclopropyl-2-(4-trifluoromethyl-phenyl)-pyrimidine-5-carbonyl]-amino}-ethoxy)-2-methyl-phenoxy]-2-methyl-propionic acid,
2-methyl-2-[2-methyl-4-(2-{[4-trifluoromethyl-2-(4-trifluoromethyl-phenyl)-pyrimidine-5-carbonyl]-amino}-ethoxy)-phenoxy]-propionic acid,
2-[4-(2-{[4-methoxymethyl-2-(4-trifluoromethyl-phenyl)-pyrimidine-5-carbonyl]-amino}-ethoxy)-2-methyl-phenoxy]-2-methyl-propionic acid,
2-[4-(2-{2-[4-cyclopropyl-2-(4-trifluoromethyl-phenyl)-pyrimidin-5-yl]-acetylamino}-ethoxy)-2-methyl-phenoxy]-2-methyl-propionic acid,
2-methyl-2-(2-methyl-4-{2-[(4′-trifluoromethyl-biphenyl-4-carbonyl)-amino]-ethoxy}-phenoxy)-propionic acid,
2-methyl-2-(2-methyl-4-{2-[(3′-trifluoromethyl-biphenyl-4-carbonyl)-amino]-ethoxy}-phenoxy)-propionic acid,
2-[4-(2-{[4-cyclopropyl-2-(3-trifluoromethyl-phenyl)-pyrimidine-5-carbonyl]-amino}-ethoxy)-2-methyl-phenoxy]-2-methyl-propionic acid,
[rac]-2-[4-(2-{[4-cyclopropyl-2-(4-trifluoromethyl-phenyl)-pyrimidine-5-carbonyl]-amino}-1-methyl-ethoxy)-2-methyl-phenoxy]-2-methyl-propionic acid,
[rac]-2-[4-(2-{[4-cyclopropyl-2-(3-trifluoromethyl-phenyl)-pyrimidine-5-carbonyl]-amino}-1]-methyl-ethoxy)-2-methyl-phenoxy]-2-methyl-propionic acid,
[rac]-2-methyl-2-(2-methyl-4-{1-methyl-2-[(3′-trifluoromethyl-biphenyl-4-carbonyl)-amino]-ethoxy}-phenoxy)-propionic acid,
[rac]-2-methyl-2-(2-methyl-4-{1-methyl-2-[(4′-trifluoromethyl-biphenyl-4-carbonyl)-amino]-ethoxy}-phenoxy)-propionic acid,
[rac]-2-[4-(2-{[4-cyclopropyl-2-(4-trifluoromethyl-phenyl)-pyrimidine-5-carbonyl]-amino}-propyl)-2-methyl-phenoxy]-2-methyl-propionic acid,
[rac]-2-[4-(2-{[4-cyclopropyl-2-(3-trifluoromethyl-phenyl)-pyrimidine-5-carbonyl]-amino}-propyl)-2-methyl-phenoxy]-2-methyl-propionic acid,
[rac]-2-methyl-2-(2-methyl-4-{2-[(3′-trifluoromethyl-biphenyl-4-carbonyl)-amino]-propyl}-phenoxy)-propionic acid,
[rac]-2-methyl-2-(2-methyl-4-{2-[(4′-trifluoromethyl-biphenyl-4-carbonyl)-amino]-propyl}-phenoxy)-propionic acid,
[rac]-2-[4-(1-{[4-cyclopropyl-2-(4-trifluoromethyl-phenyl)-pyrimidine-5-carbonyl]-amino}-propyl)-2-methyl-phenoxy]-2-methyl-propionic acid,
[rac]-2-[4-(1-{[4-cyclopropyl-2-(3-trifluoromethyl-phenyl)-pyrimidine-5-carbonyl]-amino}-propyl)-2-methyl-phenoxy]-2-methyl-propionic acid,
[rac]-2-methyl-2-(2-methyl-4-{1-[(3′-trifluoromethyl-biphenyl-4-carbonyl)-amino]-propyl}-phenoxy)-propionic acid,
[rac]-2-methyl-2-(2-methyl-4-{1-[(4′-trifluoromethyl-biphenyl-4-carbonyl)-amino]-propyl}-phenoxy)-propionic acid,
[rac]-2-methyl-2-(2-methyl-4-{1-methyl-2-[2-methyl-6-(4-trifluoromethyl-phenyl)-pyridin-3-ylcarbamoyl]-ethyl}-phenoxy)-propionic acid,
[rac]-2-(4-{2-[4-cyclopropyl-2-(4-trifluoromethyl-phenyl)-pyrimidin-5-ylcarbamoyl]-1-methyl-ethyl}-2-methyl-phenoxy)-2-methyl-propionic acid,
[rac]-2-methyl-2-{2-methyl-4-[1-methyl-2-(4′-trifluoromethyl-biphenyl-4-ylcarbamoyl)-ethyl]-phenoxy}-propionic acid,
[rac]-2-methyl-2-{2-methyl-4-[1-methyl-2-(3′-trifluoromethyl-biphenyl-4-ylcarbamoyl)-ethyl]-phenoxy}-propionic acid,
[rac]-2-(4-{1-[4-cyclopropyl-2-(4-trifluoromethyl-phenyl)-pyrimidin-5-ylcarbamoyl]-ethyl}-2-methyl-phenoxy)-2-methyl-propionic acid,
[rac]-2-methyl-2-(2-methyl-4-{1-[2-methyl-6-(4-trifluoromethyl-phenyl)-pyridin-3-ylcarbamoyl]-ethyl}-phenoxy)-propionic acid,
[rac]-2-methyl-2-{2-methyl-4-[1-(4′-trifluoromethyl-biphenyl-4-ylcarbamoyl)-ethyl]-phenoxy}-propionic acid,
[rac]-2-methyl-2-{2-methyl-4-[1-(3′-trifluoromethyl-biphenyl-4-ylcarbamoyl)-ethyl]-phenoxy}-propionic acid,
[rac]-2-methyl-2-[2-methyl-4-(1-{[2-(4-trifluoromethoxy-phenyl)-4-trifluoromethyl-pyrimidine-5-carbonyl]-amino}-ethyl)-phenoxy]-propionic acid, and
pharmaceutically acceptable salts and/or esters thereof.

19. the compound according to claim 1, selected from the group consisting of
[rac]-2-[4-(1-{[4-cyclopropyl-2-(4-trifluoromethyl-phenyl)-pyrimidine-5-carbonyl]-amino}-ethyl)-2-methyl-phenoxy]-2-methyl-propionic acid,
[rac]-2-methyl-2-(2-methyl-4-{1-[(3′-trifluoromethyl-biphenyl-4-carbonyl)-amino]-ethyl}-phenoxy)-propionic acid,
[rac]-2-(4-{1-[4-cyclopropyl-2-(4-trifluoromethyl-phenyl)-pyrimidin-5-ylcarbamoyl]-ethoxy}-2-methyl-phenoxy)-2-methyl-propionic acid,
2-methyl-2-{2-methyl-4-[(3′-trifluoromethyl-biphenyl-4-ylcarbamoyl)-methoxy]-phenoxy}-propionic acid,
2-(4-{3-[4-cyclopropyl-2-(4-trifluoromethyl-phenyl)-pyrimidin-5-ylcarbamoyl]-propyl}-phenoxy)-2-methyl-propionic acid,
2-[4-(2-{[4-cyclopropyl-2-(3-trifluoromethyl-phenyl)-pyrimidine-5-carbonyl]-amino}-ethoxy)-2-methyl-phenoxy]-2-methyl-propionic acid,
[rac]-2-[4-(2-{[4-cyclopropyl-2-(4-trifluoromethyl-phenyl)-pyrimidine-5-carbonyl]-amino}-propyl)-2-methyl-phenoxy]-2-methyl-propionic acid,
[rac]-2-methyl-2-(2-methyl-4-{1-[(4′-trifluoromethyl-biphenyl-4-carbonyl)-amino]-propyl}-phenoxy)-propionic acid,
[rac]-2-methyl-2-(2-methyl-4-{1-methyl-2-[2-methyl-6-(4-trifluoromethyl-phenyl)-pyridin-3-ylcarbamoyl]-ethyl}-phenoxy)-propionic acid, and
[rac]-2-methyl-2-{2-methyl-4-[1-(3′-trifluoromethyl-biphenyl-4-ylcarbamoyl)-ethyl]-phenoxy}-propionic acid,
and pharmaceutically acceptable salts and/or esters thereof.

20. A process for the manufacture of compounds according to claim 1, comprising the steps
a) reacting a compound of formula 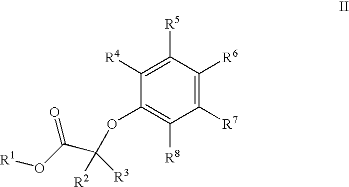
wherein R¹ is C₁₋₇-alkyl, R² to R⁸ are as defined above and one of R⁵, R⁶ or R⁷
is —X¹—COOH,
with a compound of formula 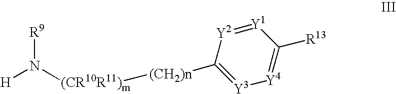
wherein Y¹ to Y⁴, R⁹, R¹⁰, R¹¹, R³, m and n are as defined in claim 1,
to obtain a compound of formula 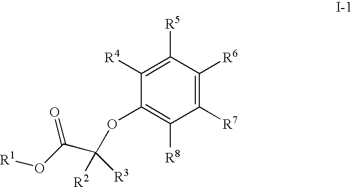
wherein one of R⁵, R⁶ and R⁷ is 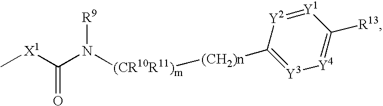
and wherein R¹ is C₁₋₇-alkyl and X¹, Y¹ to Y⁴, R² to R¹³ and m and n are as defined in claim 1, and optionally hydrolyzing the ester group to obtain a compound of formula I-1, wherein R¹ is hydrogen;
or, alternatively,
b) reacting a compound of formula 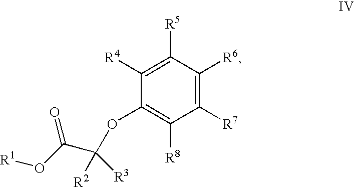
wherein R¹ is C₁₋₇-alkyl, R² to R⁸ are as defined in claim 1 and one of R⁵, R⁶ or R⁷ is —X¹—NHR⁹, wherein X¹ and R⁹ are as defined in claim 1,
with a compound of formula 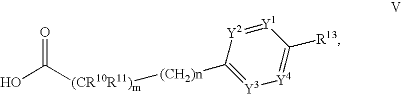
wherein Y¹ to Y⁴, R¹⁰, R¹³, m and n are as defined above,
to obtain a compound of formula 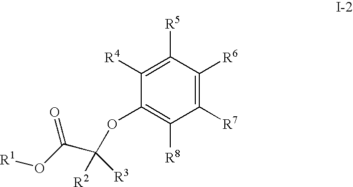
wherein one of R⁵, R⁶ and R⁷ is 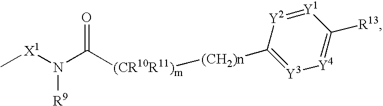
and wherein R¹ is C₁₋₇-alkyl and X¹, Y¹ to Y⁴, R² to R¹³ and m and n are as defined in claim 1, and optionally hydrolyzing the ester group to obtain a compound of formula I-2, wherein R¹ is hydrogen.

21. A pharmaceutical composition, comprising a therapeutically effective amount of a compound according to claim 1 and a pharmaceutically acceptable carrier and/or adjuvant.

22. A method for the treatment and/or prevention of diseases which are modulated by PPARδ and/or PPARα agonists, which method comprising the step of administering a therapeutically effective amount of a compound according to claim 1 to a human being or animal in need thereof.

23. The method according to claim 22, wherein said diseases are diabetes, non-insulin dependent diabetes mellitus, increased lipid and cholesterol levels, low HDL-cholesterol, high LDL-cholesterol, high triglyceride levels, atherosclerotic diseases, metabolic syndrome, syndrome X, obesity, elevated blood pressure, endothelial dysfunction, procoagulant state, dyslipidemia, polycystic ovary syndrome, inflammatory diseases or proliferative diseases.
